(19) United States
(12) Patent Application Publication
(10) Pub. No.: US 2023/0365667 A1
(43) Pub. Date: Nov. 16, 2023

(54) BINDING PROTEINS AND ANTIGEN BINDING FRAGMENTS THEREOF THAT BIND ABETA

(71) Applicant: Merck Sharpe & Dohme LLC, Rahway, NJ (US)

(72) Inventors: Alexander Mario Sevy, San Diego, CA (US); Ming-Tang Chen, Dover, MA (US)

(73) Assignee: Merck Sharp & Dohme LLC, Rahway, NJ (US)

(21) Appl. No.: 18/245,563

(22) PCT Filed: Sep. 27, 2021
(86) PCT No.: PCT/US2021/052120

§ 371 Date: Mar. 16, 2023

Related U.S. Application Data
(60) Provisional application No. 63/085,549, filed on Sep. 30, 2020.

(51) Int. Cl. C07K 16/18

(52) U.S. Cl. CPC C07K 16/18; C07K 2317/22; C07K 2317/569; C07K 2317/92; C07K 2317/34; C07K 2317/24; C07K 2317/76

(57) ABSTRACT

Binding proteins that bind amyloid beta (Abeta) are described, including heavy chain antibody variable domain (VHH) constructs comprising human-like VHH comprising three synthetically generated complementarity determining region (CDR) areas. Human-like VHHs identified using these libraries may be useful for the manufacture of therapeutics for treating diseases and disorders.

### CROSS-REFERENCE TO RELATED APPLICATIONS

This application claims priority to U.S. Provisional Application No. 63/085,549 filed Sep. 30, 2020.

### FIELD OF THE INVENTION

The present invention relates to a binding protein or antigen binding fragment thereof comprising at least one variable region that binds amyloid beta. The variable region in various embodiments comprises a complementarity determining region (CDR) or multiple CDRs (i.e., CDR1, CDR2, and CDR3) framework. Also provided are methods for binding amyloid beta and treating neuropathies using the binding protein or antigen binding fragment thereof.

### REFERENCE TO SEQUENCE LISTING SUBMITTED ELECTRONICALLY

The sequence listing of the present application is submitted electronically via EFS-Web as an ASCII formatted sequence listing with a file name “25099WOPCT-SEQLIST-07JUL2021.txt”, with a creation date of Jul. 7, 2021, and a size of 81 KB. This sequence listing submitted via EFS-Web is part of the specification and is herein incorporated by reference in its entirety.

### BACKGROUND

Amyloid precursor protein (APP) is an integral membrane protein expressed in man tissues, especially in the synapses of neurons, which plays a central role in neuronal disease pathogenesis. APP consists of a single membrane-spanning domain, a large extracellular glycosylated N-terminus and a shorter cytoplasmic C-terminus. It is one of three members of a larger gene family in humans. The other two family members are the APP-related proteins (APLPs) APLP1 and APLP26. Chen et al, 2017 Acta Pharmacologica Sinica vol. 38, pages 1205-1235.

Amyloid beta (Abeta or Aβ) is a peptide derived from amyloid precursor protein (APP) by β- and γ-secretases. Abeta is produced in the brain throughout life and accumulates in the cerebral cortex in the elderly and to an excessive degree in Alzheimer's disease. See Weller et al., 2009 Encyclopedia of Neuroscience, pages 355-362. One of the routes for elimination of Abeta is the interstitial fluid drainage pathway along basement membranes of capillary and artery walls—effectively the perivascular lymphatic drainage pathway for the brain. However, it is still unclear to what degree Abeta peptides can be cleared by enzymatic degradation, by efflux via brain drainage pathways, by active clearance by glial cells, or immunotherapy directed toward the removal of accumulating amyloid peptides. Different enzymes capable of degrading Abeta have been identified, including neprilysin, insulin-degrading enzyme (IDE), and angiotensin-converting enzyme. See Preclinical models of Alzheimer's disease for identification and preclinical validation of therapeutic targets from fine-tuning strategies for validated targets to new venues for therapy; chapter 5 (pages 115.156) found in Disease-Modifying Targets in Neurodegenerative Disorders Paving the Way for Disease-Modifying Therapies 2017.

Abeta along with other peptides and proteins such as hyperphosphorylated tau protein, is found in neurofibrillary tangles (NFTs) and amyloid plaques which are markers of neuropathies (e.g., Alzheimer's disease). Several different Aβ species have been identified that are 36-43 amino acids in length, including Aβ38, Aβ40, and Aβ42, Aβ40 peptide is the most abundant (−80-90%), followed by Aβ42 (−5-10%). The longer forms of Aβ, particularly Aβ42, are more hydrophobic and fibrillogenic (Selkoe (2001) Neuron 32(2):177-80). It is unclear what the physiological and pathological forms of Abeta are and by what mechanism Abeta causes neurological diseases and conditions, such as dementia.

Effective methods and agents that modulate Abeta are needed. These agents would allow for binding Abeta in brain tissues for the study of different neuropathies, and also for more effective treatment of neurological diseases that affect millions of patients.

### SUMMARY

The present disclosure provides methods, pharmaceutical compositions, uses and kits of binding to Abeta using a binding agent or antigen binding fragment thereof.

The present disclosure provides a binding protein (e.g., an isolated or recombinant binding protein) or antigen binding fragment thereof which specifically binds to Abeta comprising at least one variable region/domain. In various embodiments, the binding protein or antigen binding fragment thereof comprises a camelid (also referred to as a single-domain antibody, sdAb, or heavy chain antibody variable domain, V_{H}H). In various embodiments, the binding protein or antigen binding fragment thereof comprises an antibody comprising a heavy chain variable region and a light chain variable region.

In various embodiments, the variable region comprises complementarity determining regions (CDRs). For example, the binding protein or antigen binding fragment thereof comprises three heavy chain CDRs, i.e., CDR1, CDR2, and CDR3. In various embodiments of the binding protein or antigen binding fragment thereof, the three CDRs are selected from the group consisting of:
(i) a variable region comprising CDR1, CDR2, and CDR3 regions comprising the amino acid sequences of SEQ ID NOs:143, 144, and 145, respectively
(ii) a variable region comprising CDR1, CDR2, and CDR3 regions comprising the amino acid sequences of SEQ ID NOs:147, 148, and 149, respectively; and
(iii) a variable region comprising CDR1, CDR2, and CDR3 regions comprising the amino acid sequences of SEQ ID NOs:151, 152, and 153, respectively.

In various embodiments, the at least one CDR (either the CDR1, CDR2 or CDR3) in the binding protein or antigen binding fragment thereof is selected from a CDR comprising an amino acid sequence listed in Table 4. In various embodiments, at least one CDR is selected from a CDR comprising an amino acid sequence listed in Table 6. For example, at least one of CDR1, CDR2, or CDR3 comprises an amino acid sequence listed in Table 4 or Table 6.

In various embodiments, the variable region comprises the CDR1, CDR2, and CDR3 regions comprising the amino acid sequences of SEQ ID NOs:143, 144, and 145, respectively.

In various embodiments, the variable region comprises the CDR1, CDR2, and CDR3 regions comprising the amino acid sequences of SEQ ID NOs:147, 148, and 149, respectively.

In various embodiments, the variable region comprises the variable region comprising CDR1, CDR2, and CDR3 regions comprising the amino acid sequences of SEQ ID NOs:151, 152, and 153, respectively.

In various embodiments, the binding protein or antigen binding fragment thereof comprises a CDR1 comprising an amino acid sequence selected from the group consisting of SEQ ID NOs: 2, 6, 10, 14, 18, 22, 26, 30, 34, 38, 42, 46, 50, 54, 58, 62, 66, 70, 74, 78, 82, 86, 90, 94, 98, 102, 106, 110, 114, 118, 122, 126, 130, 134, and 138.

In various embodiments, the binding protein or antigen binding fragment thereof comprises a CDR1 which is at least 75%, 80%, 85%, 90%, 95%, 98%, or 99% identical to an amino acid sequence selected from the group consisting of SEQ ID NOs: 2, 6, 10, 14, 18, 22, 26, 30, 34, 38, 42, 46, 50, 54, 58, 62, 66, 70, 74, 78, 82, 86, 90, 94, 98, 102, 106, 110, 114, 118, 122, 126, 130, 134, and 138.

In various embodiments, the binding protein or antigen binding fragment thereof comprises a CDR2 comprising an amino acid sequence selected from the group consisting of SEQ ID NOs: 3, 7, 11, 15, 19, 23, 27, 31, 35, 39, 43, 47, 51, 55, 59, 63, 67, 71, 75, 79, 83, 87, 91, 95, 99, 103, 107, 111, 115, 119, 123, 127, 131, 135, and 139.

In various embodiments, the binding protein or antigen binding fragment thereof comprises a CDR2 which is at least 75%, 80%, 85%, 90%, 95%, 98%, or 99% identical to an amino acid sequence selected from the group consisting of SEQ ID NOs: 3, 7, 11, 15, 19, 23, 27, 31, 35, 39, 43, 47, 51, 55, 59, 63, 67, 71, 75, 79, 83, 87, 91, 95, 99, 103, 107, 111, 115, 119, 123, 127, 131, 135, and 139.

In various embodiments, the binding protein or antigen binding fragment thereof comprises a CDR3 comprising an amino acid sequence selected from the group consisting of SEQ ID NOs: 4, 8, 12, 16, 20, 24, 28, 32, 36, 40, 44, 48, 52, 56, 60, 64, 68, 72, 76, 80, 84, 88, 92, 96, 100, 104, 108, 112, 116, 120, 124, 128, 132, 136, and 140.

In various embodiments, the binding protein or antigen binding fragment thereof comprises a CDR3 which is at least 75%, 80%, 85%, 90%, 95%, 98%, or 99% identical to an amino acid sequence selected from the group consisting of SEQ ID NOs: 4, 8, 12, 16, 20, 24, 28, 32, 36, 40, 44, 48, 52, 56, 60, 64, 68, 72, 76, 80, 84, 88, 92, 96, 100, 104, 108, 112, 116, 120, 124, 128, 132, 136, and 140.

In various embodiments, the binding protein or antigen binding fragment thereof comprises a variable region comprising three heavy chain complementarity determining regions (CDRs) selected from the group consisting of:
(a) a variable region comprising CDR1, CDR2, and CDR3 regions comprising the amino acid sequences of SEQ ID NOs: 2, 3, and 4, respectively;
(b) a variable region comprising CDR1, CDR2, and CDR3 regions comprising the amino acid sequences of SEQ ID NOs: 6, 7, and 8, respectively;
(c) a variable region comprising CDR1, CDR2, and CDR3 regions comprising the amino acid sequences of SEQ ID NOs: 10, 11, and 12, respectively;
(d) a variable region comprising CDR1, CDR2, and CDR3 regions comprising the amino acid sequences of SEQ ID NOs: 14, 15, and 16, respectively;
(e) a variable region comprising CDR1, CDR2, and CDR3 regions comprising the amino acid sequences of SEQ ID NOs: 18, 19, and 20, respectively;
(f) a variable region comprising CDR1, CDR2, and CDR3 regions comprising the amino acid sequences of SEQ ID NOs: 22, 23, and 24, respectively
(g) a variable region comprising CDR1, CDR2, and CDR3 regions comprising the amino acid sequences of SEQ ID NOs: 26, 27, and 28, respectively;
(h) a variable region comprising CDR1, CDR2, and CDR3 regions comprising the amino acid sequences of SEQ ID NOs: 30, 31, and 32, respectively;
(i) a variable region comprising CDR1, CDR2, and CDR3 regions comprising the amino acid sequences of SEQ ID NOs: 34, 35, and 36, respectively;
(j) a variable region comprising CDR1, CDR2, and CDR3 regions comprising the amino acid sequences of SEQ ID NOs: 38, 39, and 40, respectively;
(k) a variable region comprising CDR1, CDR2, and CDR3 regions comprising the amino acid sequences of SEQ ID NOs: 42, 43, and 44, respectively;
(l) a variable region comprising CDR1. CDR2, and CDR3 regions comprising the amino acid sequences of SEQ ID NOs: 46, 47, and 48, respectively;
(m) a variable region comprising CDR1, CDR2, and CDR3 regions comprising the amino acid sequences of SEQ ID NOs: 50, 51, and 52, respectively;
(n) a variable region comprising CDR1, CDR2, and CDR3 regions comprising the amino acid sequences of SEQ ID NOs: 54, 55, and 56, respectively;
(o) a variable region comprising CDR1, CDR2, and CDR3 regions comprising the amino acid sequences of SEQ ID NOs: 58, 59, and 60, respectively;
(p) a variable region comprising CDR1, CDR2, and CDR3 regions comprising the amino acid sequences of SEQ ID NOs: 62, 63, and 64, respectively;
(q) a variable region comprising CDR1, CDR2, and CDR3 regions comprising the amino acid sequences of SEQ ID NOs: 66, 67, and 68, respectively;
(r) a variable region comprising CDR1, CDR2, and CDR3 regions comprising the amino acid sequences of SEQ ID NOs: 70, 71, and 72, respectively;
(s) a variable region comprising CDR1, CDR2, and CDR3 regions comprising the amino acid sequences of SEQ ID NOs: 74, 75, and 76, respectively;
(t) a variable region comprising CDR1, CDR2, and CDR3 regions comprising the amino acid sequences of SEQ ID NOs: 78, 79, and 80, respectively;
(u) a variable region comprising CDR1, CDR2, and CDR3 regions comprising the amino acid sequences of SEQ ID NOs: 82, 83, and 84, respectively;
(v) a variable region comprising CDR1, CDR2, and CDR3 regions comprising the amino acid sequences of SEQ ID NOs: 86, 87, and 88, respectively;
(w) a variable region comprising CDR1, CDR2, and CDR3 regions comprising the amino acid sequences of SEQ ID NOs: 90, 91, and 92, respectively;
(x) a variable region comprising CDR1, CDR2, and CDR3 regions comprising the amino acid sequences of SEQ ID NOs: 94, 95, and 96, respectively;
(y) a variable region comprising CDR1, CDR2, and CDR3 regions comprising the amino acid sequences of SEQ ID NOs: 98, 99, and 100, respectively;
(z) a variable region comprising CDR1, CDR2, and CDR3 regions comprising the amino acid sequences of SEQ ID NOs: 102, 103, and 104, respectively;
(aa) a variable region comprising CDR1, CDR2, and CDR3 regions comprising the amino acid sequences of SEQ ID NOs: 106, 107, and 108, respectively;
(bb) a variable region comprising CDR1, CDR2, and CDR3 regions comprising the amino acid sequences of SEQ ID NOs: 110, 111, and 112, respectively;
(cc) a variable region comprising CDR1, CDR2, and CDR3 regions comprising the amino acid sequences of SEQ ID NOs: 114, 115, and 116, respectively;
(dd) a variable region comprising CDR1, CDR2, and CDR3 regions comprising the amino acid sequences of SEQ ID NOs: 118, 119, and 120, respectively;
(ee) a variable region comprising CDR1, CDR2, and CDR3 regions comprising the amino acid sequences of SEQ ID NOs: 122, 123, and 124, respectively;
(ff) a variable region comprising CDR1, CDR2, and CDR3 regions comprising the amino acid sequences of SEQ ID NOs: 126, 127, and 128, respectively;
(gg) a variable region comprising CDR1, CDR2, and CDR3 regions comprising the amino acid sequences of SEQ ID NOs: 130, 131, and 132, respectively;
(hh) a variable region comprising CDR1, CDR2, and CDR3 regions comprising the amino acid sequences of SEQ ID NOs: 134, 135, and 136, respectively; and
(ii) a variable region comprising CDR1, CDR2, and CDR3 regions comprising the amino acid sequences of SEQ ID NOs: 138, 139, and 140.

In various embodiments, the binding protein or antigen binding fragment thereof comprises a variable region sequence selected from the group consisting of SEQ ID NOs: 5, 9, 13, 17, 21, 25, 29, 33, 37, 41, 45, 49, 53, 57, 61, 65, 69, 73, 77, 81, 85, 89, 93, 97, 101, 105, 109, 113, 117, 121, 125, 129, 133, 137, and 141.

In various embodiments, the binding protein or antigen binding fragment thereof comprises a variable region sequence which is at least 85%, 90%, 95%, 98%, or 99% identical to an amino acid sequence selected from the group consisting of SEQ ID NOs: 5, 9, 13, 17, 21, 25, 29, 33, 37, 41, 45, 49, 53, 57, 61, 65, 69, 73, 77, 81, 85, 89, 93, 97, 101, 105, 109, 113, 117, 121, 125, 129, 133, 137, and 141.

In various embodiments, the binding protein or antigen binding fragment thereof comprises a variable region sequence comprising an amino acid sequence selected from the group consisting of SEQ ID NOs: 142, 146, and 150.

In various embodiments, the binding protein or antigen binding fragment thereof comprises a variable region sequence which is at least 85%, 90%, 95%, 98%, or 99% identical to the amino acid sequence selected from the group consisting of SEQ ID NOs: 142, 146, and 150.

In various embodiments, the binding protein or antigen binding fragment thereof comprises a variable region sequence comprising the amino acid sequence of SEQ ID NO: 5.

In various embodiments, the binding protein or antigen binding fragment thereof comprises a variable region sequence comprising the amino acid sequence of SEQ ID NO: 9.

In various embodiments, the binding protein or antigen binding fragment thereof comprises a variable region sequence comprising the amino acid sequence of SEQ ID NO: 13.

In various embodiments, the binding protein or antigen binding fragment thereof comprises a variable region sequence comprising the amino acid sequence of SEQ ID NO: 17.

In various embodiments, the binding protein or antigen binding fragment thereof comprises a variable region sequence comprising the amino acid sequence of SEQ ID NO: 21.

In various embodiments, the binding protein or antigen binding fragment thereof comprises a variable region sequence comprising the amino acid sequence of SEQ ID NO: 25.

In various embodiments, the binding protein or antigen binding fragment thereof comprises a variable region sequence comprising the amino acid sequence of SEQ ID NO: 29.

In various embodiments, the binding protein or antigen binding fragment thereof comprises a variable region sequence comprising the amino acid sequence of SEQ ID NO: 33.

In various embodiments, the binding protein or antigen binding fragment thereof comprises a variable region sequence comprising the amino acid sequence of SEQ ID NO 37.

In various embodiments, the binding protein or antigen binding fragment thereof comprises a variable region sequence comprising the amino acid sequence of SEQ ID NO: 41.

In various embodiments, the binding protein or antigen binding fragment thereof comprises a variable region sequence comprising the amino acid sequence of SEQ ID NO: 45.

In various embodiments, the binding protein or antigen binding fragment thereof comprises a variable region sequence comprising the amino acid sequence of SEQ ID NO: 49.

In various embodiments, the binding protein or antigen binding fragment thereof comprises a variable region sequence comprising the amino acid sequence of SEQ ID NO: 53.

In various embodiments, the binding protein or antigen binding fragment thereof comprises a variable region sequence comprising the amino acid sequence of SEQ ID NO: 57.

In various embodiments, the binding protein or antigen binding fragment thereof comprises a variable region sequence comprising the amino acid sequence of SEQ ID NO: 61.

In various embodiments, the binding protein or antigen binding fragment thereof comprises a variable region sequence comprising the amino acid sequence of SEQ ID NO: 65.

In various embodiments, the binding protein or antigen binding fragment thereof comprises a variable region sequence comprising the amino acid sequence of SEQ ID NO: 69.

In various embodiments, the binding protein or antigen binding fragment thereof comprises a variable region sequence comprising the amino acid sequence of SEQ ID NO: 73.

In various embodiments, the binding protein or antigen binding fragment thereof comprises a variable region sequence comprising the amino acid sequence of SEQ ID NO: 77.

In various embodiments, the binding protein or antigen binding fragment thereof comprises a variable region sequence comprising the amino acid sequence of SEQ ID NO: 81.

In various embodiments, the binding protein or antigen binding fragment thereof comprises a variable region sequence comprising the amino acid sequence of SEQ ID NO: 85.

In various embodiments, the binding protein or antigen binding fragment thereof comprises a variable region sequence comprising the amino acid sequence of SEQ ID NO: 89.

In various embodiments, the binding protein or antigen binding fragment thereof comprises a variable region sequence comprising the amino acid sequence of SEQ ID NO: 93

In various embodiments, the binding protein or antigen binding fragment thereof comprises a variable region sequence comprising the amino acid sequence of SEQ ID NO: 97.

In various embodiments, the binding protein or antigen binding fragment thereof comprises a variable region sequence comprising the amino acid sequence of SEQ ID NO: 101.

In various embodiments, the binding protein or antigen binding fragment thereof comprises a variable region sequence comprising the amino acid sequence of SEQ ID NO: 105.

In various embodiments, the binding protein or antigen binding fragment thereof comprises a variable region sequence comprising the amino acid sequence of SEQ ID NO: 109.

In various embodiments, the binding protein or antigen binding fragment thereof comprises a variable region sequence comprising the amino acid sequence of SEQ ID NO: 113.

In various embodiments, the binding protein or antigen binding fragment thereof comprises a variable region sequence comprising the amino acid sequence of SEQ ID NO: 117.

In various embodiments, the binding protein or antigen binding fragment thereof comprises a variable region sequence comprising the amino acid sequence of SEQ ID NO: 121.

In various embodiments, the binding protein or antigen binding fragment thereof comprises a variable region sequence comprising the amino acid sequence of SEQ ID NO: 125.

In various embodiments, the binding protein or antigen binding fragment thereof comprises a variable region sequence comprising the amino acid sequence of SEQ ID NO: 129.

In various embodiments, the binding protein or antigen binding fragment thereof comprises a variable region sequence comprising the amino acid sequence of SEQ ID NO: 133.

In various embodiments, the binding protein or antigen binding fragment thereof comprises a variable region sequence comprising the amino acid sequence of SEQ ID NO: 137.

In various embodiments, the binding protein or antigen binding fragment thereof comprises a variable region sequence comprising the amino acid sequence of SEQ ID NO: 141.

In various embodiments, the binding protein or antigen binding fragment thereof comprises a variable region sequence selected from the group consisting of SEQ ID NOs: 142, 146 and 150.

In various embodiments, the binding protein or antigen binding fragment thereof comprises a variable region sequence which is at least 85%, 90%, 95%, 98%, or 99% identical to an amino acid sequence selected from the group consisting of SEQ ID NOs: 142, 146 and 150.

In various embodiments, the binding protein or antigen binding fragment thereof comprises a variable region sequence comprising the amino acid sequence of SEQ ID NO: 142.

In various embodiments, the binding protein or antigen binding fragment thereof comprises a variable region sequence comprising the amino acid sequence of SEQ ID NO: 146.

In various embodiments, the binding protein or antigen binding fragment thereof comprises a variable region sequence comprising the amino acid sequence of SEQ ID NO: 150.

In various embodiments, the binding protein or antigen binding fragment thereof comprises binds to mammalian Abeta. For example, the mammalian Abeta is a human Abeta. For example, the human Abeta comprises a sequence comprising about 36 to about 43 amino acids. In various embodiments the human Abeta comprises the amino acid sequence selected from the group consisting of SEQ ID NOs: 1, and 154-158. In various embodiments the human Abeta comprises the amino acid sequence of SEQ ID NO: 1.

In various embodiments, the binding protein or antigen binding fragment thereof binds soluble Abeta. In various embodiments, the binding protein or antigen binding fragment thereof binds Abeta oligomers and/or Abeta in a tangled form. In various embodiments, the binding protein or antigen binding fragment thereof binds Abeta on brain plaques. In various embodiments, the binding protein or antigen binding fragment thereof inhibits Abeta aggregation, and/or binds to Abeta in aggregated form. In various embodiments, the binding protein or antigen binding fragment thereof inhibits formulation of a plaque comprising Abeta. In various embodiments, the binding protein or antigen binding fragment thereof inhibits formation of plaques associated with a neuropathy. For example, the neuropathy is Alzheimer's disease, dementia, or Parkinson's disease.

In various embodiments, the binding protein or antigen binding fragment thereof binds to human Abeta with a K_{D} as described in Table 5. In various embodiments, the binding protein or antigen binding fragment thereof binds to human Abeta with a K_{D} of 50 nanomolar (nM) or less, 40 nM or less, 30 nM or less, 20 nM or less, 10 nM or less, 9 nM or less, 8 nM or less, 7 nM or less, 6 nM or less, or 5 nM or less. In various embodiments, the binding is determined by KinExA or Biacore™.

In various embodiments, the binding protein or antigen binding fragment thereof binds to Abeta in vitro.

In various embodiments, the binding protein or antigen binding fragment thereof to Abeta in vivo.

In various embodiments, the binding protein or antigen binding fragment thereof binds to mammalian Abeta. For example, binding protein or antigen binding fragment thereof binds to murine Abeta. In various embodiments, the binding protein or antigen binding fragment thereof binds to a human Abeta. In various embodiments, the binding protein or antigen binding fragment thereof binds to a human Abeta comprising about 38 amino acids to about 43 amino acids. In various embodiments the human Abeta comprises the amino acid sequence selected from the group consisting of SEQ ID NOs: 1, and 154-158. In various embodiments, the binding protein or antigen binding fragment thereof binds to a human Abeta comprising the amino acid sequence of SEQ ID NO: 1. In various embodiments, the binding protein or antigen binding fragment thereof binds to soluble Abeta and/or Abeta in aggregated form.

In various embodiments, the binding protein or antigen binding fragment thereof comprises a heavy chain antibody variable domain, V_{H}H, or antigen binding fragment thereof.

In various embodiments, the binding protein or antigen binding fragment thereof comprises an antibody or antigen binding fragment thereof. In various embodiments, the antibody is a chimeric, human or humanized antibody.

Also provided herein is a binding protein or antigen binding fragment thereof which binds to the same epitope on Abeta as the binding protein or antigen binding fragment thereof of any of the previous embodiments or described herein.

In various embodiments, the binding protein or antigen binding fragment thereof binds to an epitope of human Abeta. In various embodiments the human Abeta comprises the amino acid sequence selected from the group consisting of SEQ ID NOs: 1, and 154-158. For example, the Abeta comprises the amino acid sequence of SEQ ID NO: 1.

Also provided herein is an binding protein or antigen binding fragment thereof described herein (e.g., in Table 4 and Table 6) or of any of the preceding embodiments which binds to one or more residues of residues 1-16, 8-17, and/or 17-40 of human Abeta (SEQ ID NO: 1). In various embodiments, the binding protein or antigen binding fragment thereof binds to an epitope of human Abeta comprising amino acid residues 1-16 of SEQ ID NO: 1. In various embodiments, the binding protein or antigen binding fragment thereof binds to an epitope of human Abeta comprising amino acid residues 8-17 of SEQ ID NO: 1. In various embodiments, the binding protein or antigen binding fragment thereof binds to an epitope of human Abeta comprises amino acid residues 17-40 of SEQ ID NO: 1.

Also provided herein is an bispecific molecule comprising the binding protein or antigen binding fragment thereof described herein (e.g., Table 4 or Table 6) of any of the preceding embodiments, wherein the binding protein or antigen binding fragment thereof is linked to a molecule having a second binding region.

In various embodiments of the bispecific molecule, the second binding region binds to a tumor-associated antigen. In various embodiments of the bispecific molecule, the second binding region binds to an antigen or ligand associated with a neurological disease.

Also provided herein is an immunoconjugate comprising:
a binding protein or antigen binding fragment thereof described herein (e.g., in Table 4 and Table 6) or of any of the preceding embodiments or in the various embodiments or the bispecific molecule described herein or of any of the preceding embodiments; and a moiety selected from the group consisting of a detectable moiety, a binding moiety, a labeling moiety, or a biologically active moiety.

Also provided herein is a nucleic acid comprising a nucleotide sequence that encodes the heavy chain variable region of at least one of the binding proteins or antigen binding fragments thereof described herein (e.g., in Table 4 and Table 6) or of any of the preceding embodiments or at least one of the bispecific molecules described herein or of any of the preceding embodiments.

Also provided herein is an expression vector comprising at least one of the nucleic acids described herein.

Also provided herein is a cell transformed with at least one of the expression vectors described herein.

Also provided herein is a pharmaceutical composition comprising:
at least one of the binding proteins or antigen binding fragments thereof (e.g., in Table 4 and Table 6), bispecific molecules, or immunoconjugates described herein or of any of the preceding embodiments; and
a pharmaceutically acceptable carrier.

In various embodiments, the pharmaceutical composition further comprises one or more additional therapeutic agents. In various embodiments of the pharmaceutical composition, the one or more additional therapeutic agents is selected from the group consisting of an anti-cancer agent, a chemotherapeutic agent, an immunosuppressive agent, an immunostimulatory agent, an anti-inflammatory agent, and an immune checkpoint inhibitor.

In various embodiments of the pharmaceutical composition, the one or more additional therapeutic agents is selected from the group consisting of an cholinesterase inhibitor (e.g., such as donepezil, galantamine, rivastigmine, and tacrine), NMDA receptor antagonist (e.g., memantine), amyloid beta peptide aggregation inhibitor, antioxidant, gamma-secretase modulator, nerve growth factor (NGF) mimic, NGF gene therapy, PPAR agonist (e.g., PPAR-alpha agonist and PPAR-gamma agonist), HMS-CoA reductase inhibitor (e.g., statins), ampakine, calcium channel blocker, GABA receptor antagonist, glycogen synthase kinase inhibitor, intravenous immunoglobulin, muscarinic receptor agonist, nicotinic receptor modulator, active or passive amyloid beta peptide for immunization, phosphodiesterase inhibitor, serotonin receptor antagonist, anti-amyloid beta peptide antibody, growth hormone, neurotrophic factor, brain-derived neurotrophic factor (BDNF), nerve growth factor (NGF), neurotrophin-4/5, fibroblast growth factor (FGF)-2 and other FGFs, neurotrophin (NT)-3, erythropoietin (EPO), hepatocyte growth factor (HGF), epidermal growth factor (EGF), transforming growth factor (TGF)-al ha, TGF-beta, vascular endothelial growth factor (VEGF), interleukin-1 receptor antagonist (IL-1ra), ciliary neurotrophic factor (CNTF), glial-derived neurotrophic factor (GDNF), neurturin, platelet-derived growth factor (PDGF), heregulin, neuregulin, artemin, persephin, interleukins, glial cell line derived neurotrophic factor (GFR), granulocyte-colony stimulating factor (CSF), granulocyte-macrophage-CSF, netrin, cardiotrophin-1, hedgehog, leukemia inhibitory factor (LIF), midkine, pleiotrophin, a bone morphogenetic protein (BMP), netrins, saposin, semaphorin, stem cell factor (SCF), and a different anti-Abeta antibody.

In certain embodiments, the one or more additional therapeutic agent is selected from the following: an imaging agent, a cytotoxic agent, an angiogenesis inhibitor, a kinase inhibitor, a co-stimulation molecule blocker, an adhesion molecule blocker, an anti-cytokine antibody or functional fragment thereof, a detectable label or reporter, an antirheumatic, a muscle relaxant, a narcotic, a non-steroid anti-inflammatory drug (NSAID), an analgesic, an anesthetic, a sedative, a neuromuscular blocker, an antimicrobial, an anti-psoriatic, a corticosteroid, an anabolic steroid, an erythropoietin, an immunoglobulin, an immunosuppressive, a growth hormone, a hormone replacement drug, a radiopharmaceutical, an antidepressant, an antipsychotic, a stimulant, an asthma medication, a beta agonist, an inhaled steroid, an epinephrine or analog, a cytokine, and a cytokine antagonist. In an embodiment, the composition co-administered with the binding protein can be selected from the following: budenoside, epidermal growth factor, a corticosteroid, cyclosporin, sulfasalazine, an aminosalicylate, 6-mercaptopurine, azathioprine, metronidazole, a lipoxygenase inhibitor, mesalamine, olsalazine, balsalazide, an antioxidant, a thromboxane inhibitor, a growth factor, an elastase inhibitor, a pyridinyl-imidazole compound, an antibody, antagonist or agonist of TNF, LT, IL-1, IL-1 R, IL-2, IL-4, IL-6, IL-6R, IL-7, IL-8, IL-10, IL-11, IL-12, IL-13, IL-15, IL-16, IL-18, IL-23, TGF-β, EMAP-II, GM-CSF, FGF, PDGF, CD2, CD3, CD4, CD8, CD-19, CD25, CD28, CD30, CD40, CD45, CD69, CD90 or a ligand thereof, methotrexate, FK506, rapamycin, mycophenolate mofetil, leflunomide, ibuprofen, prednisolone, a phosphodiesterase inhibitor, an adenosine agonist, an antithrombotic agent, a complement inhibitor, an adrenergic agent, IRAK, NIK, IKK, p38, a MAP kinase inhibitor, an IL-1 β converting enzyme inhibitor, a TNFα-converting enzyme inhibitor, a T-cell signaling inhibitor, a metalloproteinase inhibitor, an angiotensin converting enzyme inhibitor, a soluble cytokine receptor, a soluble p55 TNF receptor, a soluble p75 TNF receptor, slL-1 Rl, slL-1 Rll, slL-6R and combinations thereof.

In various embodiments of the pharmaceutical composition, the one or more additional therapeutic agents is selected from the group consisting of a tau degrader, a tau aggregation inhibitor, a tau vaccine, an alpha synuclein (α-synuclein) degrader, α-synuclein aggregation inhibitor, α-synuclein vaccine, a TAR-DNA binding protein 43 (TDP-43) degrader, a TDP-43 aggregation inhibitor, an apolipoprotein E (APOE or ApoE) degrader, an ApoE aggregation inhibitor, an ApoE ligand binding agonist, an ApoE ligand binding antagonist, an ApoE receptor modulator, a triggering receptor expressed on myeloid cells 2 (Trem2) receptor agonist, a nuclear factor erythroid 2-related factor 2 (NRF2) activator, a NUAK family SNF1-like kinase 1 (NUAK1) inhibitor, a tau tubulin kinase 1 (TTBK1) inhibitor, a NLR family pyrin domain containing 3 (NLRP3) inhibitor, a receptor-interacting serine/threonine-protein kinase 1 (RIPK1) inhibitor, a nicotinamide adenine dinucleotide phosphate (NADPH) oxidase 2 (Nox2) modulator, a proteosome modulator, a transient receptor potential mucolipin 1 (TRPML1), a proteo-lipid dysfunction/aggregation modulator, a metabotropic glutamate receptor 2 (mGluR2) modulator, a α7 nicotinic acetylcholine receptor modulator, and a phosphodiesterase type 10 (PDE10) inhibitor.

Also provided herein is a kit comprising at least one of the binding proteins or antigen binding fragments thereof (e.g., in Table 4 and Table 6), bispecific molecules, immunoconjugates, or pharmaceutical compositions described herein or in any of the preceding embodiments; and instructions for use.

Also provided herein is a method of producing a binding protein or antigen binding fragment thereof described herein comprising:
culturing a host cell comprising a polynucleotide encoding the amino acid sequences (e.g., comprising the heavy chain variable region) of at least one of the binding proteins or antigen binding fragments thereof described herein (e.g., in Table 4 or Table 6) or in any of the preceding embodiments under conditions favorable to expression of the polynucleotide; and optionally, recovering the binding protein or antigen binding fragment thereof from the host cell and/or culture medium.

Also provided herein is a method of selectively binding Abeta on a cell, neural structure, and/or extracellular deposit comprising administering to the cell, neural structure, and/or extracellular deposit at least one of the binding proteins or antigen binding fragments thereof (e.g., in Table 4 and Table 6), bispecific molecules, immunoconjugates, or pharmaceutical compositions described herein or in any of the preceding embodiments. In various embodiments, the cell, neural structure, and/or extracellular deposit is from a sample from a subject. In various embodiments, the cell, neural structure, and/or extracellular deposit is in a subject in need thereof.

In various embodiments of the method, the cell is a neuronal cell. In various embodiments of the method, the extracellular deposit comprises a brain plaque.

Also provided herein is a method of treating a neurological disorder or condition associated comprising administering to a subject in need thereof a therapeutically effective amount of at least one of the binding proteins or antigen binding fragments thereof (e.g., in Table 4 and Table 6), bispecific molecules, immunoconjugates, or pharmaceutical compositions described herein or in any of the preceding embodiments.

Also provided herein is a method of inhibiting Abeta associated with a neurological disorder or condition comprising administering to a subject in need thereof a therapeutically effective amount of at least one of the binding proteins or antigen binding fragments thereof (e.g., in Table 4 and Table 6), bispecific molecules, immunoconjugates, or pharmaceutical compositions described herein or in any of the preceding embodiments.

In various embodiments of the method, the neurological disorder or condition associated with Abeta is selected from the group consisting of familial Alzheimer's disease, sporadic Alzheimer's disease, frontotemporal dementia with parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis, a parkinsonism-dementia complex, Down syndrome, Gerstmann-Straussler-Scheinker disease, Hallervorden-Spatz disease, inclusion body myositis, Creutzfeld-Jakob disease, multiple system atrophy, Niemann-Pick disease type C, prion protein cerebral amyloid angiopathy, subacute sclerosing panencephalitis, myotonic dystrophy, non-Guamanian motor neuron disease with neurofibrillary tangles, postencephalitic parkinsonism, chronic traumatic encephalopathy, and dementia pugulistica (boxing disease).

In various embodiments, the methods further comprise administering one or more additional therapies or treatments. In certain embodiments, the one or more additional therapeutic agent is selected from the following: an imaging agent, a cytotoxic agent, an angiogenesis inhibitor, a kinase inhibitor, a co-stimulation molecule blocker, an adhesion molecule blocker, an anti-cytokine antibody or functional fragment thereof, a detectable label or reporter, an antirheumatic, a muscle relaxant, a narcotic, a NSAID, an analgesic, an anesthetic, a sedative, a neuromuscular blocker, an antimicrobial, an anti-psoriatic, a corticosteroid, an anabolic steroid, an erythropoietin, an immunoglobulin, an immunosuppressive, a growth hormone, a hormone replacement drug, a radiopharmaceutical, an antidepressant, an antipsychotic, a stimulant, an asthma medication, a beta agonist, an inhaled steroid, an epinephrine or analog, a cytokine, and a cytokine antagonist. In an embodiment, the composition co-administered with the binding protein can be selected from the following: budenoside, epidermal growth factor, a corticosteroid, cyclosporin, sulfasalazine, an aminosalicylate, 6-mercaptopurine, azathioprine, metronidazole, a lipoxygenase inhibitor, mesalamine, olsalazine, balsalazide, an antioxidant, a thromboxane inhibitor, a growth factor, an elastase inhibitor, a pyridinyl-imidazole compound, an antibody, antagonist or agonist of TNF, LT, IL-1, IL-1 R, IL-2, IL-4, IL-6, IL-6R, IL-7, IL-8, IL-10, IL-11, IL-12, IL-13, IL-15, IL-16, IL-18, IL-23, TGF-β, EMAP-II, GM-CSF, FGF, PDGF, CD2, CD3, CD4, CD8, CD-19, CD25, CD28, CD30, CD40, CD45, CD69, CD90 or a ligand thereof, methotrexate, FK506, rapamycin, mycophenolate mofetil, leflunomide, ibuprofen, prednisolone, a phosphodiesterase inhibitor, an adenosine agonist, an antithrombotic agent, a complement inhibitor, an adrenergic agent, IRAK. NIK, IKK, p38, a MAP kinase inhibitor, an IL-1 β converting enzyme inhibitor, a TNFα-converting enzyme inhibitor, a T-cell signaling inhibitor, a metalloproteinase inhibitor, an angiotensin converting enzyme inhibitor, a soluble cytokine receptor, a soluble p55 TNF receptor, a soluble p75 TNF receptor, slL-1 Rl, slL-1 Rll, slL-6R and combinations thereof.

In various embodiments of the pharmaceutical composition, the one or more additional therapeutic agents is selected from the group consisting of a tau degrader, a tau aggregation inhibitor, a tau vaccine, an α-synuclein degrader, α-synuclein aggregation inhibitor, α-synuclein vaccine a TDP-43 degrader, a TDP-43 aggregation inhibitor, an ApoE degrader, an ApoE aggregation inhibitor, an ApoE ligand binding agonist, an ApoE ligand binding antagonist, an ApoE receptor modulator, a Trem2 receptor agonist, a NRF2 activator, a NUAK1 inhibitor, a TTBK1 inhibitor, a NLRP3 inhibitor, a rRIPK1) inhibitor, a Nox2 modulator, a proteosome modulator, a TRPML1, a proteo-lipid dysfunction/aggregation modulator, a mGluR2 modulator, a α7 nicotinic acetylcholine receptor modulator, and a phosphodiesterase type 10 (PDE10) inhibitor.

In other embodiments, the one or additional therapies or treatments is capable of modulating a biological function of one or more targets associated with a neurological disease or condition. In certain aspects of this embodiment, the one or more additional therapies or treatments specifically binds to an epitope, antigen, receptor or target, such that a biological function is modulated. In certain embodiments, one or more additional therapies or treatments binds to the binding receptors expressed on the brain vascular endothelium as well as a therapeutic target. In these embodiments, the epitope, antigen, receptor or target can be selected from CGRP, TNFα, RGMA, Substance P, Bradykinin, Nav1.7, LPA, P2X3, NGF, Abeta; APP, BACE1; IL-1 β; IGF1, or 2; IL-18; IL-6; RAGE; NGF; EGFR; cMet; Her2; RGMA, and CD-20.

Another aspect of the invention provides at least one of the binding proteins or antigen binding fragments thereof (e.g., in Table 4 and Table 6), bispecific molecules, immunoconjugates, or pharmaceutical compositions described herein or in any of the preceding embodiments, for use in the preparation of a medicament to: bind Abeta; inhibit Abeta; and/or treat a neurological disorder or condition associated with Abeta.

In various embodiments, the neurological disorder or condition associated with Abeta is selected from the group consisting of familial Alzheimer's disease, sporadic Alzheimer's disease, frontotemporal dementia with parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis, a parkinsonism-dementia complex, Down syndrome, Gerstmann-Straussler-Scheinker disease, Hallervorden-Spatz disease, inclusion body myosins, Creutzfeld-Jakob disease, multiple system atrophy, Niemann-Pick disease type C, prion protein cerebral amyloid angiopathy, subacute sclerosing panencephalitis, myotonic dystrophy, non-Guamanian motor neuron disease with neurofibrillary tangles, postencephalitic parkinsonism, chronic traumatic encephalopathy, and dementia pugulistica (boxing disease).

Another aspect of the invention provides the use of at least one of the binding proteins or antigen binding fragments thereof (e.g., in Table 4 and Table 6), bispecific molecules, immunoconjugates, or pharmaceutical compositions described herein or in any of the preceding embodiments for the manufacture of a medicament for: binding Abeta; inhibiting Abeta; and/or treating a neurological disorder or condition associated with Abeta.

In various embodiments of the use, the neurological disorder or condition associated with Abeta is selected from the group consisting of familial Alzheimer's disease, sporadic Alzheimer's disease, frontotemporal dementia with parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis, a parkinsonism-dementia complex, Down syndrome, Gerstmann-Straussler-Scheinker disease, Hallervorden-Spatz disease, inclusion body myositis, Creutzfeld-Jakob disease, multiple system atrophy, Niemann-Pick disease type C, prion protein cerebral amyloid angiopathy, subacute sclerosing panencephalitis, myotonic dystrophy, non-Guamanian motor neuron disease with neurofibrillary tangles, postencephalitic parkinsonism, chronic traumatic encephalopathy, and dementia pugulistica (boxing disease).

Another aspect of the invention provides a method of detecting the presence of Abeta in a sample comprising contacting the sample with at least one of the binding proteins or antigen binding fragments thereof (e.g., in Table 4 and Table 6), bispecific molecules, immunoconjugates, or pharmaceutical compositions described herein or in any of the preceding embodiments, under conditions that allow for binding between Abeta and the at least one of the binding proteins or antigen binding fragments thereof (e.g., in Table 4 and Table 6), bispecific molecules, immunoconjugates, or pharmaceutical compositions described herein or in any of the preceding embodiments, and detecting the binding.

In various embodiments of the method, the sample comprises a biological sample from a subject. In various embodiments of the method, the subject has neurological disorder or condition.

### BRIEF DESCRIPTION OF THE DRAWINGS

FIG. 1A shows by illustration the yeast display cytometry detection and selection of binding proteins that bind Abeta described in the working examples.

FIG. 1B is a representative flow cytometry plot of output after multiple rounds of FACS selection. The X-axis shows biotinylated Abeta antigen binding, and the Y-axis shows V_{H}H expression, as detected by the strategy of FIG. 1A.

FIG. 2A, FIG. 2B, FIG. 2C, and FIG. 2D shows flow cytometry plots of output after multiple rounds of FACS selection for libraries vhhLib-001, vhhLib-002 and vhhLib-003. Clones from each library displayed different binding affinity profiles with affinities ranging from 0 to about 50 nM. Binding to different Abeta peptides (i.e., 1-40, 1-16 and 5-20 of SEQ ID NO: 1) were analyzed.

FIG. 3A, FIG. 3B, FIG. 3C, and FIG. 3D are photographs of CRND8 mouse tissues stained using the different Abeta binding V_{H}Hs described herein. Specifically, 4-month CRND8 mice tissues were collected and incubated for 32 minutes with the biotinylated V_{H}Hs with or without formic acid. During the incubation some of the samples/biotinylated V_{H}Hs were or some were not heated (37° C.). Note that for FIG. 3A, FIG. 3B, FIG. 3C, and FIG. 3D vhhLib02-53 corresponds to 06BHK, vhhLib02-91 corresponds to 09BHK; vhhLib05-8 corresponds to 13BHK; vhhLib05-21 corresponds to 16BHK, and corresponds to vhhLib05-49 20BHK in Tables 4-6.

### DETAILED DESCRIPTION

Neurological diseases and conditions affect millions of patients and their causes are not fully understood due to the involvement of age, genetics and environmental factors. Alzheimer's disease is a neurodegenerative disorder associated with progressive memory loss and cognitive dysfunction. The disease is characterized by the presence in the brain of amyloid plaques, comprised of Abeta protein, and neurofibrillary tangles (NFTs), comprised of hyperphosphorylated tau protein.

Alzheimer's Disease (referred to as AD) is a degenerative brain disorder presented clinically by progressive loss of memory, cognition, reasoning, judgment, and emotional stability that gradually leads to profound mental deterioration and ultimately death. Individuals with AD exhibit characteristic β-amyloid deposits, i.e., β-amyloid plaques or fibrils, in the brain and in cerebral blood vessels, as well as neurofibrillary tangles in areas of the human brain important for memory and cognitive function, as determined during post-mortem analysis of AD patients' brains.

β-amyloid aggregates of different stages, ranging from fibrils (as seen in β-amyloid plaques), protofibrils, oligomers, amyloid pores. Aβ*56 and AD diffusible ligands (ADDL) are predominantly composed of β-amyloid peptides or fragments of β-amyloid peptides, including those ranging in length from 38-43 residues, i.e., Aβ1-38, Aβ1-39, Aβ1-40, Aβ1-42, Aβ1-43 peptides, and fragments thereof, which are interchangeably referred to herein as AB peptides and Aβ peptides. The amino acid sequences of the Aβ peptides are known and differ only in the amino acids present at the C-terminus. For example, the sequence of the Aβ1-42 and Aβ1-43 differs from that of the Aβ1-40 peptide by the addition of two and three, respectively, amino acids at the carboxyl (COOH) terminus. See U.S. patent publication number 20110071301.

Aβ peptide aggregate deposits (e.g., plaques) are also characterized in the brains of individuals with Down's Syndrome (Trisomy 21); mixed dementia, including those with combined AD and Parkinson's disease features and those with Lewy body diseases; cerebral amyloid angiopathy, Hereditary Cerebral Hemorrhage with Beta amyloidosis of the Dutch-Type, homozygotes for the apolipoprotein E4, inclusion body myositis, Niemann-Pick type C disease, and other such disorders.

Parkinson's disease (PD) is a progressive neurodegenerative disease affecting 1-2% of the population over 65 years of age. It has been estimated that the number of cases of PD worldwide will double by the year 2030. Currently, there is no cure, early detection mechanism, preventative treatment, or effective way to slow disease progression. Classic neuronal pathological features of PD include the loss of dopaminergic (DA) neurons in the substantia nigra (SN) and the presence of cytoplasmic inclusions, known as Lewy bodies. Classic clinical features of PD include resting tremor, bradykinesia and rigidity, but the disease also leads to a wide variety of non-motor features such as autonomic dysfunction and dementia. The majority of PD patients suffer from idiopathic disease with no clear etiology, and approximately 5% of patients present with familial PD. Although the pattern of neuronal loss in PD is well characterized, the molecular mechanisms that lead to cell death are still unknown. See WO2015191934A2. Existing treatments of these neurological diseases and conditions cannot stop their progression, let alone cure the disease. A major hurdle to treating these diseases is the ability to inhibit presence of neurotoxic Aβ plaques in the brain, which requires effectively binding and modulating Abeta. The invention provides a binding protein (e.g., antibodies and V_{H}Hs) or antigen binding fragment that binds to Abeta.

Monoclonal antibody therapeutics have seen tremendous growth in recent years, with the number of approved antibody therapeutics nearly tripling between 2010 and 2019 (Kaplon et al. MAbs 12, (2020)). In addition to the traditional full-length IgG format, there has been sustained interest in developing single-domain antibody (sdAb) therapeutics as well. Such single-domain formats include human heavy-chain only antibodies (Rouet et al., J. Biol. Chem. 290, 11905-11917 (2015); To et al., J. Biol. Chem. 280, 41395-41403 (2005)), camelid V_{H}H (Hamers-Casterman et al., Nature 363, 446-448 (1993); Muyldermans, Annu. Rev. Biochem. 82, 775-797 (2013)) and shark VNAR (Ubah et al., Biochem. Soc. Trans. 46, 1559-1565 (2018); Wesolowski et al., Med. Microbiol. Immunol. 198, 157-174 (2009)) as well as engineered formats not naturally produced by any organism (Saerens et al., Curr. Opin. Pharmacol. 8, 600-608 (2008); Vazquez-Lombardi et al., Drug Discov. Today 20, 1271-1283 (2015)). Among these a format of particular interest is camelid V_{H}H, which has the following advantages: 1) small size, 2) ease of production, 3) sequence similarity to human antibodies, minimizing immunogenicity, and 4) modularity that allows domains to be combined to form multi-specifics. Recently V_{H}H have been developed to combat infectious diseases (Sarker et al., Gastroenterol. 145, 740-748.e8 (2013); Laursen et al., Science 362, 598-602 (2018)) and the first V_{H}H was caplacizumab for acquired thrombotic thrombocytopenic purpura (aTTP) approved by the FDA for human use in 2019 (Morrison, Nat. Rev. Drug Discov. 18, 485-487 (2019)) with multiple V_{H}H currently in clinical trials (Kaplon et al., Op. Cit.; Iezzi et al., Frontiers in Immunology (2018). doi:10.3389/fimmu.2018.002731).

Currently the most common method for generating V_{H}H is by animal immunization with the antigen of interest and isolation of antigen-specific B cells. This approach can be challenging, given that animal immunization is expensive, time-consuming, and not amenable to all antigen types (i.e., antigens unstable at 37° C. for prolonged periods of time). In addition, there is no control over human likeness or developability of the lead molecules, as well as the fact that not all antibodies recovered from an animal are V_{H}H.

### Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

The amyloid-beta peptide, which is also termed “amyloid β”, “Aβ”, “Aβ4” or “β-A4” is a main component of the extracellular neuritic plaques that are associated with amyloidogenic diseases such as Alzheimer's disease. Amyloid β is derived from “Alzheimer precursor protein/β-amyloid precursor protein” (APP). APPs are integral membrane glycoproteins (see Sisodia (1992), PNAS Vol. 89, pp. 6075) and are endoproteolytically cleaved within the Abeta sequence by a plasma membrane protease, α-secretase (see Sisodia (1992), loc. cit.). Furthermore, further secretase activity, in particular β-secretase and γ-secretase activity, leads to the extracellular release of amyloid-β (Aβ) comprising either 39 amino acids (Aβ39), 40 amino acids (Aβ40), 42 amino acids (Aβ42) or 43 amino acids (Aβ43); see Sinha (1999), PNAS 96, 11094-1053; Price (1998), Science 282, 1078 to 1083; WO 00/72880 or Hardy (1997), TINS 20, 154. See also U.S. Pat. No. 7,879,976, Selkoe (1994), Ann. Rev. Cell Biol. 10, 373-403, Koo (1999), PNAS Vol. 96, pp. 9989-9990, U.S. Pat. No. 4,666,829 or Glenner (1984), BBRC 12, 1131.

Aβ has several naturally occurring forms, whereby the human forms are referred to as the above mentioned Aβ39, Aβ40, Aβ41, Aβ42 and Aβ43. A common form, Aβ42, has the amino acid sequence (starting from the N-terminus):

(SEQ ID NO: 154)
DAEFRHDSGYEVHHQKLVFFAEDVGSNKGAIIGLMVGGVVIA.

In Aβ41, Aβ40, Aβ39, the C-terminal amino acids A, IA and VIA are missing, respectively. In the Aβ43-form an additional threonine residue is comprised at the C-terminus of the above depicted sequence (SEQ ID NO: 154).

Aβ41
(SEQ ID NO: 155)
DAEFRHDSGYEVHHQKLVFFAEDVGSNKGAIIGLMVGGWI
Aβ40
(SEQ ID NO: 156)
DAEFRHDSGYEVHHQKLVFFAEDVGSNKGAIIGLMVGGW
Aβ39
(SEQ ID NO: 157)
DAEFRHDSGYEVHHQKLVFFAEDVGSNKGAIIGLMVGGV
Aβ43
(SEQ ID NO: 158)
DAEFRHDSGYEVHHQKLVFFAEDVGSNKGAIIGLMVGGVVIAT

The term “affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (KD). Affinity can be measured by common methods known in the art, including KinExA and Biacore™. Specific illustrative and exemplary embodiments for measuring binding affinity are described in the following.

The term “administration” and “treatment,” as it applies to an animal, human, experimental subject, cell, tissue, organ, or biological fluid, refers to contact of an exogenous pharmaceutical, therapeutic, diagnostic agent, or composition comprising a V_{H}H as described herein to the animal, human, subject, cell, tissue, organ, or biological fluid. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell. The term “subject” includes any organism, preferably an animal, more preferably a mammal (e.g., human, rat, mouse, dog, cat, rabbit). In a preferred embodiment, the term “subjects” refers to a human.

The term “amino acid” refers to a simple organic compound containing both a carboxyl (—COOH) and an amino (—NH₂) group. Amino acids are the building blocks for proteins, polypeptides, and peptides. Amino acids occur in L-form and D-form, with the L-form in naturally occurring proteins, polypeptides, and peptides. Amino acids and their code names are set forth in the following chart.

| | Three letter | One letter |
|---|---|---|
| Amino acid | code | code |
| Alanine | Ala | A |
| Arginine | Arg | R |
| Asparagine | Asn | N |
| Aspartic acid | Asp | D |
| Cysteine | Cvs | C |
| Glutamine | Gln | Q |
| Glutamic acid | Glu | E |
| Glycine | Glv | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tvr | Y |
| Valine | Val | V |

The term “antibody” or “immunoglobulin” as used herein refers to a glycoprotein comprising either (a) at least two heave chains (HCs) and two light chains (LCs) inter-connected by disulfide bonds, or (b) in the case of a species of camelid antibody, at least two heavy chains (HCs) inter-connected by disulfide bonds. Each HC is comprised of a heavy chain variable region or domain NH) and a heavy chain constant region or domain. In certain naturally occurring IgG, IgD and IgA antibodies, the heavy chain constant region is comprised of three domains, C_{H}1, C_{H}2 and C_{H}3. In general, the basic antibody structural unit for antibodies is a tetramer comprising two HC/LC pairs, except for the species of camelid antibodies comprising only two HCs, in which case the structural unit is a homodimer. Each tetramer includes two identical pairs of polypeptide chains, each pair having one LC (about 25 kDa) and HC chain (about 50-70 kDa).

In certain naturally occurring antibodies, each light chain is comprised of an LC variable region or domain (V_{L}) and a LC constant domain. The LC constant domain is comprised of one domain, C_{L}. The human Vii includes seven family members: V_{H}1, V_{H}2, V_{H}3, V_{H}4, V_{H}5, V_{H}6, and V_{H}7; and the human V_{L} includes 16 family members: V_{κ}1, V_{κ}2, V_{κ}3, V_{κ}4, V_{κ}5, V_{κ}6, V_{λ}1, V_{λ}2, V_{λ}3, V_{λ}4, V_{λ}5, V_{λ}6, V_{λ}7, V_{λ}8, V_{λ}9, and V_{λ}10. Each of these family members can be further divided into particular subtypes. The V_{H} and V_{L} domains can be further subdivided into regions of hypervariability, termed complementarity determining region (CDR) areas, interspersed with regions that are more conserved, termed framework regions (FR). Each V_{H} and V_{L} is composed of three CDR regions and four FR regions, arranged from amino-terminus to carboxy-terminus in the following order: FR1, CDR1, FR2, CDR2, FR3, CDR3, FR4. Numbering of the amino acids in a V_{H} or V_{H}H may be determined using Kabat numbering scheme. See Béranger, et al., Ed. Ginetoux, Correspondence between the IMGT unique numbering for C-DOMAIN, the IMGT exon numbering, the Eu and Kabat numberings: Human IGHG, Created: 17 May 2001, Version: 8 Jun. 2016, which is accessible at www.imgt.org/IMGTScientificChart/Numbering/Hu_IGHGnber.html).

The constant regions of the antibodies may mediate the binding of the immunoglobulin to host tissues or factors, including various cells of the immune system (e.g., effector cells) and the first component (C1q) of the classical complement system. Typically, the numbering of the amino acids in the heavy chain constant domain begins with number 118, which is in accordance with the Eu numbering scheme. The Eu numbering scheme is based upon the amino acid sequence of human IgG₁ (Eu), which has a constant domain that begins at amino acid position 118 of the amino acid sequence of the IgG₁ described in Edelman et al., Proc. Natl. Acad. Sci. USA. 63: 78-85 (1969), and is shown for the IgG₁, IgG₂, IgG₃, and IgG₄ constant domains in Béranger, et al., Ibid.

The variable regions of the heavy and light chains contain a binding domain comprising the CDRs that interacts with an antigen. A number of methods are available in the art for defining CDR sequences of antibody variable domains (see Dondelinger et al., Frontiers in Immunol. 9: Article 2278 (2018)). The common numbering schemes include the following.
Kabat numbering scheme is based on sequence variability and is the most commonly used (See Kabat et al. Sequences of Proteins of Immunological Interest, 5th Ed. Public Health Service, National Institutes of Health, Bethesda, Md. (1991) (defining the CDR regions of an antibody by sequence);
Chothia numbering scheme is based on the location of the structural loop region (See Chothia & Lesk J. Mol. Biol. 196: 901-917 (1987); Al-Lazikani et al., J. Mol. Biol. 273: 927-948 (1997));
AbM numbering scheme is a compromise between the two used by Oxford Molecular's AbM antibody modelling software (see Karu et al, ILAR Journal 37: 132-141 (1995);
Contact numbering scheme is based on an analysis of the available complex crystal structures (See www.bioinf.org.uk:Prof. Andrew C. R. Martin's Group; Abhinandan & Martin, Mol. Immunol. 45:3832-3839 (2008).
IMGT (ImMunoGeneTics) numbering scheme is a standardized numbering system for all the protein sequences of the immunoglobulin superfamily, including variable domains from antibody light and heavy chains as well as T cell receptor chains from different species and counts residues continuously from 1 to 128 based on the germ-line V sequence alignment (see Giudicelli et al., Nucleic Acids Res. 25:206-11 (1997); Lefranc, Immunol Today 18:509(1997); Lefranc et al., Dev Comp Immunol. 27:55-77 (2003)).

The following general rules disclosed in www.bioinf.org.uk:Prof Andrew C. R. Martin's Group and reproduced in Table 1 below may be used to define the CDRs in an antibody sequence that includes those amino acids that specifically interact with the amino acids comprising the epitope in the antigen to which the antibody binds. There are rare examples where these generally constant features do not occur; however, the Cys residues are the most conserved feature.

TABLE 1
CDR sequences using different numbering schemes

| Loop | Kabat | AbM | Chothia¹ | Contact² | IMGT |
|---|---|---|---|---|---|
| L1 | L24--L34 | L24--L34 | L24--L34 | L30--L36 | L27--L32 |
| L2 | L50--L56 | L50--L56 | L50--L56 | L46--L55 | L50--L52 |
| L3 | L89--L97 | L89--L97 | L89--L97 | L89--L96 | L89--L97 |
| H1 | H31--H35B | H26--H35B | H26--H32 . . . 34 | H30--H35B | H26--H35B |
| | (Kabat Numbering)³ | | | | |
| H1 | H31--H35 | H26--H35 | H26--H32 | H30--H35 | H26--H33 |
| | (Chothia Numbering) | | | | |
| H2 | H50--H65 | H50--H58 | H52--H56 | H47--H58 | H51--H56 |
| H3 | H95--H102 | H95--H102 | H95--H102 | H93--H101 | H93--H102 |

¹Some of these numbering schemes (particularly for Chothia loops) vary depending on the individual publication examined.
²Any of the numbering schemes can be used for these CDR definitions, except the Contact numbering scheme uses the Chothia or Martin (Enhanced Chothia) definition.
³The end of the Chothia CDR-H1 loop when numbered using the Kabat numbering convention varies between H32 and H34 depending on the length of the loop. (This is because the Kabat numbering scheme places the insertions at H35A and H35B.)
If neither H35A nor H35B is present, the loop ends at H32
If only H35A is present, the loop ends at H33
If both H35A and H3SB are present, the loop ends at H34

In general, the state of the art recognizes that in many cases, the CDR3 region of the heavy chain is the primary determinant of antibody specificity, and examples of specific antibody generation based on CDR3 of the heavy chain alone are known in the art (e.g., Beiboer et al., J. Mol. Biol. 296: 833-849 (2000); Klimka et al., British J. Cancer 83: 252-260 (2000); Rader et al., Proc. Natl. Acad. Sci. USA 95: 8910-8915 (1998); Xu et al., Immunity 13: 37-45 (2000).

The term “antigen” as used herein refers to any foreign substance which induces an immune response in the body.

The term “binding protein” means any protein or peptide that binds to a ligand, such as an antigen or epitope of Abeta.

The term “bispecific molecule” means an molecule (e.g., antibody) that binds one antigen (or epitope) on one of its two binding arms (one pair of HC/LC), and binds a different antigen (or epitope) on its second binding arm (a different pair of HC/LC). A bispecific antibody has two distinct antigen binding arms (in both specificity and CDR sequences), and is monovalent for each antigen to which it binds. Bispecific antibodies include those generated by quadroma technology (Milstein and Cuello (1983) Nature 305(5934): 537-40), by chemical conjugation of two different monoclonal antibodies (Staerz et al. (1985) Nature 314(6012): 628-31), or by knob-into-hole or similar approaches which introduces mutations in the Fc region (Holliger et al. (1993) Proc. Natl. Acad. Sci. USA 90(14): 6444-6448), among others.

The term “camelized” V_{H} refers to an ISVD in which one or more amino acid residues in the amino acid sequence of a naturally occurring V_{H} domain from a conventional four-chain antibody by one or more of the amino acid residues that occur at the corresponding position(s) in a V_{H}H domain of a heavy chain antibody. Such “camelizing” substitutions may be inserted at amino acid positions that form and/or are present at the V_{H}-V_{L} interface, and/or at the so-called Camelidae hallmark residues, as defined herein (see also for example WO9404678 and Davies and Riechmann (1994 and 1996)). Reference is made to Davies and Riechmann (FEBS 339: 285-290, 1994; Biotechnol. 13: 475-479, 1995; Prot. Eng. 9: 531-537, 1996) and Riechmann and Muyldermans (J. Immunol. Methods 231: 25-38, 1999).

The terms “cell,” “cell line,” and “cell culture” are used interchangeably and all such designations include progeny. Thus, the words “transformants” and “transformed cells” include the primary subject cell and cultures derived therefrom without regard for the number of transfers. It is also understood that not all progeny will have precisely identical DNA content, due to deliberate or inadvertent mutations. Mutant progeny that have the same function or biological activity as screened for in the originally transformed cell are included. Where distinct designations are intended, it will be clear from the context.

The term “CDR area” refers to a CDR as defined by any one of the methods commonly used for defining CDRs and which may further include up to one amino acid N-terminal to the defined CDR or up to three amino acids C-terminal to the defined CDR.

The term “control sequences” or “regulatory sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to use promoters, polyadenylation signals, and enhancers.

A nucleic acid is “operably linked” when it is placed into a functional relationship with another nucleic acid sequence. For example, DNA for a pre-sequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “epitope”, as used herein, is defined in the context of a molecular interaction between a human-like V_{H}H and its corresponding “antigen” (Ag). Generally, “epitope” refers to the area or region on an Ag to which human-like V_{H}H specifically binds, i.e., the area or region in physical contact with the human-like V_{H}H. Physical contact may be defined through distance criteria (e.g., a distance cut-off of 4 Å) for atoms in the human-like V_{H}H and Ag molecules.

The epitope for a given human-like V_{H}H/Ag pair can be defined and characterized at different levels of detail using a variety of experimental and computational epitope mapping methods. The experimental methods include mutagenesis, X-ray crystallography, Nuclear Magnetic Resonance (NMR) spectroscopy and Hydrogen deuterium exchange Mass Spectrometry (HX-MS), methods that are known in the art. As each method relies on a unique principle, the description of an epitope is intimately linked to the method by which it has been determined. Thus, depending on the epitope mapping method employed, the epitope for a given Ab/Ag pair will be described differently.

The epitope for a given human-like V_{H}H/Ag pair may be described by routine methods. For example, the overall location of an epitope may be determined by assessing the ability of the human-like V_{H}H to bind to different fragments or variants of the antigen. The specific amino acids within the antigen that make contact with an epitope may also be determined using routine methods. For example, the human-like V_{H}H and Ag molecules may be combined and the human like V_{H}H/Ag complex may be crystallized. The crystal structure of the complex may be determined and used to identify specific sites of interaction between the human-like V_{H}H and Ag.

The term “expression” as used herein is defined as the transcription and/or translation of a particular nucleotide sequence.

The term “Fc domain”, or “Fc” as used herein is the crystallizable fragment domain or region obtained from an antibody that comprises the C_{H}2 and C_{H}3 domains of an antibody. In an antibody, the two Fc domains are held together by two or more disulfide bonds and by hydrophobic interactions of the C_{H}3 domains. The Fc domain may be obtained by digesting an antibody with the protease papain.

The term “gene” is used broadly to refer to any segment of nucleic acid associated with a biological function. Thus, genes include coding sequences and/or the regulatory sequences required for their expression. For example, “gene” refers to a nucleic acid fragment that expresses mRNA, functional RNA, or specific protein, including regulatory sequences. “Genes” also include non-expressed DNA segments that, for example, form recognition sequences for other proteins. “Genes” can be obtained from a variety of sources, including cloning from a source of interest or synthesizing from known or predicted sequence information, and may include sequences designed to have desired parameters.

The term “germline” or “germline sequence” refers to a sequence of unrearranged immunoglobulin DNA sequences. Any suitable source of unrearranged immunoglobulin sequences may be used. Human germline sequences may be obtained, for example, from JOINSOLVER® germline databases on the website for the National Institute of Arthritis and Musculoskeletal and Skin Diseases of the United States National Institutes of Health. Mouse germline sequences may be obtained, for example, as described in Giudicelli et al. (2005) Nucleic Acids Res. 33:D256-D261.

The term “immunoglobulin single-chain variable domains” (abbreviated herein as “ISVD”), and interchangeably used with “single variable domain”, defines molecules wherein the antigen binding site is present on, and formed by, a single immunoglobulin domain. This sets immunoglobulin single variable domains apart from “conventional” immunoglobulins or their fragments, wherein two immunoglobulin domains, in particular two variable domains, interact to form an antigen binding site. Typically, in conventional immunoglobulins, a heavy chain variable domain (heavy chain variable domain, heavy chain variable region, and V_{H} are used interchangeably) and a light chain variable domain (light chain variable domain, light chain variable region, and V_{L}) interact to form an antigen binding site. In the latter case, the complementarity determining region (CDR) areas of both V_{H} and V_{L} will contribute to the antigen binding site, i.e., a total of six CDRs will be involved in antigen binding site formation. In view of the above definition, the antigen-binding domain of a conventional four-chain antibody (such as an IgG, IgM, IgA, IgD or IgE molecule; known in the art) or of a Fab fragment, a F(ab′)₂ fragment, an Fv fragment such as a di-sulphide linked Fv or a scFv fragment, or a diabody (all known in the art) derived from such conventional four-chain antibody, would normally not be regarded as an ISVD, as, in these cases, binding to the respective epitope of an antigen would normally not occur by one (single) immunoglobulin domain but by a pair of (associating) immunoglobulin domains such as light and heavy chain variable domains, i.e., by a V_{H}-V_{L} pair of immunoglobulin domains, which jointly bind to an epitope of the respective antigen.

In contrast, ISVDs are capable of specifically binding to an epitope of the antigen without pairing with an additional immunoglobulin variable domain. The binding site of an ISVD is formed by a single V_{H}H or V_{H} domain. Hence, the antigen binding site of an ISVD is formed by no more than three CDRs. As such, the single variable domain may be a heavy chain variable domain sequence (e.g., a V_{H}-sequence or V_{H}H sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit).

An ISVD as used herein is selected from the group consisting of V_{H}Hs, human-like V_{H}Hs, and camelized V_{H}s.

The term “mammal” means any species that is a member of the class mammalia, including rodents, primates, dogs, cats, camelids, lagomorphs and ungulates. The term “rodent” refers to any species that is a member of the order rodentia including mice, rats, hamsters, and gerbils. The term “primate” refers to any species that is a member of the order primates, including monkeys, apes and humans. The term “lagomorph” refers to any species that is a member of the order lagomorpha, including rabbits and hares. The term “ungulates” refers to any species that is a member of the superorder ungulata including cattle, horses and camelids. The term “camelid” refers to any species that is a member of the family camelidae including camels and llamas.

The term “nanobody” and “NANOBODIES” as used herein are registered trademarks of Ablynx N.V.

The term “nucleic acid molecule” refers to a polynucleotide.

The term “peptide” typically refers to a polymer composed of less than 43 amino acid residues, related naturally occurring structural variants, and synthetic non-naturally occurring analogs thereof linked via peptide bonds, related naturally occurring structural variants, and synthetic non-naturally occurring analogs thereof.

The term “polynucleotide” as used herein is defined as a chain of nucleotides. Furthermore, nucleic acid molecules are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning and amplification technology, and the like, and by synthetic means. An “oligonucleotide” as used herein refers to a short polynucleotide, typically less than 100 bases in length. RNA and DNA molecules are polynucleotides.

The term “polypeptide” refers to a polymer composed of 43 or more amino acid residues, related naturally occurring structural variants, and synthetic non-naturally occurring analogs thereof linked via peptide bonds, related naturally occurring structural variants, and synthetic non-naturally occurring analogs thereof.

The terms “promoter”, “promoter region”, or “promoter sequence” refer generally to transcriptional regulatory regions of a gene, which may be found at the 5′ or 3′ side of the coding region, or within the coding region, or within introns. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

The term “surface anchor” or “surface anchoring moiety” refers to any polypeptide or peptide that, when fused with an Fc or functional fragment thereof, is expressed and located to the cell surface where a human-like V_{H}H Fc fusion protein can form a pairwise interaction with the Fc or functional fragment thereof attached to the cell surface. An example of a cell surface anchor is a protein such as, but not limited to, SED-1, α-agglutinin, Cwp1, Cwp2, GasI, Yap3, FloIp1 Crh2, Pir1, Pir4, Tip1, Wpi, Hpwp1, Als3, and Rbt5; for example, Saccharomyces cerevisiae CWP1, CWP2, SED1, or GAS1; Pichia pastoris SP1 or GAS1; or H. polymorpha TIP1. The surface anchor further includes any polypeptide with a signal peptide that when fused to the C-terminus of the Fc or functional fragment thereof (fusion protein) to the endoplasmic reticulum (ER) where it is inserted into the ER membrane via a translocon and is attached to the ER membrane by its hydrophobic C terminus. The hydrophobic C-terminal sequence is then cleaved off and replaced by the GPI-anchor (glycosylphosphatidylinositol). As the fusion protein processes through the secretory pathway, it is transferred via vesicles to the Golgi apparatus and finally to the plasma membrane where it remains attached to a leaflet of the cell membrane.

The term “synthetically generated” with respect to CDR and CDR area sequences refers to CDR sequences which are designed using computer algorithms to identify those amino acids in each CDR or CDR area that may varied over those amino acids that are kept constant to the extent each variable amino acid may be varied. For example, the variable amino acid at a particular position in the CDR or CDR area may be any amino acid except C, or any amino acid except C and M, or any amino acid within a subset of amino acids. A plurality of RNA or DNA molecules encoding V_{H}H are then synthesized wherein each V_{H}H comprises CDRs or CDR areas having a particular combination of variable CDRs and/or CDR areas as determined using the computer algorithms. Thus, a nucleic acid molecule library is constructed in which each nucleic acid molecule independently encodes a particular V_{H}H having a particular combination of CDR and/or CDR area sequences.

The term “target of interest” refers to any molecule, protein, polypeptide, peptide, carbohydrate, nucleic acid, or any other molecule it is desired to have the human-like V_{H}H bind. In general parlance, the target of interest may be referred to as an antigen.

A cell has been “transformed”, “transduced”, or “transfected” by exogenous or heterologous DNA when such DNA has been introduced inside the cell. The introduced RNA or DNA may or may not be integrated (covalently linked) into the genome of the cell. In prokaryotes, yeast, and mammalian cells for example, the introduced DNA may be maintained on an episomal element such as a plasmid. With respect to eukaryotic cells, a stably transformed or transduced cell is one in which the introduced RNA or DNA has become integrated into a chromosome so that it is inherited by daughter cells through chromosome replication. This stability is demonstrated by the ability of the eukaryotic cell to establish cell lines or clones comprised of a population of daughter cells containing the introduced RNA or DNA. A “clone” is a population of cells derived from a single cell or common ancestor by mitosis. A “cell line” is a clone of a primary cell that is capable of stable growth in vitro for many generations.

The term “vector,” as used herein, refers to either a delivery vehicle as described herein or to a vector such as an expression vector.

The term “V_{H}H” as used herein indicates that the heavy chain variable domain is obtained from or originated or derived from a heavy chain antibody. Heavy chain antibodies are functional antibodies that have two heavy chains and no light chains. Heavy chain antibodies exist in and are obtainable from Camelids (e.g., camels and alpacas), members of the biological family Camelidae. V_{H}H antibodies, have originally been described as the antigen binding immunoglobulin (variable) domain of “heavy chain antibodies” (i.e., of “antibodies devoid of light chains”; Hamers-Casterman et al., Nature 363: 446-448 (1993). The term “V_{H}H domain” has been chosen in order to distinguish these variable domains from the heavy chain variable domains that are present in conventional four-chain antibodies (which are referred to herein as “V_{H} domains” or “V_{H}”) and from the light chain variable domains that are present in conventional four-chain antibodies (which are referred to herein as “V_{L} domains” or “V_{L}”). For a further description of V_{H}Hs, reference is made to the review article by Muyldermans (Reviews in Molec. Biotechnol. 74: 277-302, (2001), as well as to the following patent applications, which are mentioned as general background art: international patent publications numbers WO9404678, WO9504079 and WO9634103 of the Vrije Universiteit Brussels WO9425591, WO9937681, WO0040968, WO0043507, WO0065057, WO0140310, WO0144301, EP1134231 and WO0248193 of Unilever; WO9749805, WO0121817, WO03035694, WO03054016 and WO03055527 of the Vlaams Instituut voor Biotechnologie (VI B); WO03050531 of Algonomics N.V. and Ablynx N.V.; WO0190190 by the National Research Council of Canada; WO03025020 (=EP 1433793) by the Institute of Antibodies; as well as international patent publications numbers WO2004041867, WO2004041862, WO2004041865, WO2004041863, WO2004062551, WO2005044858, WO200640153, WO2006079372, WO2006122786, WO 06122787, WO2006122825, WO2008101985, WO2008142164, and WO2015173325 by Ablynx N.V. and the further published patent applications by Ablynx N.V. Reference is also made to the further prior art mentioned in these applications, and in particular to the list of references mentioned on pages 41-43 of the International application WO2006040153, which list and references are incorporated herein by reference.

Methods of obtaining V_{H}H domains binding to a specific antigen or epitope have been described earlier, e.g., in WO2006/040153 and WO2006/122786. As also described therein in detail, V_{H}H domains derived from camelids can be “humanized” or made “human-like” by being engineered, for example, by replacing one or more amino acid residues in the amino acid sequence of the original V_{H}H sequence by one or more of the amino acid residues that occur at the corresponding position(s) in a V_{H} domain from a conventional 4-chain antibody from a human being. A humanized V_{H}H domain can contain one or more fully human framework region sequences, and, in an even more specific embodiment, can contain human framework region sequences derived from DP-29, DP-47, DP-51, or parts thereof, optionally combined with JH sequences, such as JH5.

V_{H}H CDRs can be grafted into multiple types of binding proteins (e.g., antibodies) and the CDRs retain binding. When V_{H}H CDRs is grafted to a framework, it may be engineered so as to have desirable binding behavior. For example, the V_{H}H can be linked genetically to Fc-domains, other nanobodies, peptide tags, or toxins and can be conjugated chemically at a specific site to drugs, radionuclides, photosensitizers, and nanoparticles. See Bannas et al., 2017 Front Immunol.; 8: 1603. In certain embodiments of the method, the binding protein is selected from: a single-chain antibody (scFv); a recombinant camelid heavy-chain-only antibody (V_{H}H); a shark heavy-chain-only antibody (VNAR); a microprotein; a darpin; an anticalin; an adnectin; an aptamer; a Sac7d derivative (affitins, e.g., NANOFITINS, see Journal of Molecular Biology. 2008 Nov. 28; 383(5):1058-68, the contents of which are hereby incorporated by reference), a Fv; a Fab; a Fab′: and a F(ab′)2. In an embodiment, the binding protein is heterodimeric, for example the binding protein has greater potency than each individual monomer. In alternative embodiments, the heteromultimeric neutralizing binding protein is multimeric and the multimeric components are associated non-covalently or covalently.

V_{H}Hs are antibody-derived therapeutic proteins that contain the unique structural and functional properties of naturally-occurring heavy-chain antibodies. V_{H}H technology is based on fully functional antibodies from camelids that lack light chains. These heavy-chain antibodies contain a single variable domain (V_{H}H) and two constant domains (CH2 and CH3). The cloned and isolated V_{H}H domain is a stable polypeptide harboring the antigen-binding capacity of the original heavy-chain antibody. See Castorman et al. U.S. Pat. No. 5,840,526 issued Nov. 24, 1998; and Castorman et al. U.S. Pat. No. 6,015,695 issued Jan. 18, 2000, each of which is incorporated by reference herein in its entirety. V_{H}Hs are commercially available from Ablynx Inc. (Ghent, Belgium) under the trademark of NANOBODIES™.

Suitable methods of producing or isolating antibody fragments having the requisite binding specificity and affinity are described herein and include for example, methods which select recombinant antibody from a library, by PCR (See Ladner U.S. Pat. No. 5,455,030 issued Oct. 3, 1995 and Devy et al. U.S. Pat. No. 7,745,587 issued Jun. 29, 2010, each of which is incorporated by reference herein in its entirety).

Functional fragments of antibodies, including fragments of chimeric, humanized, primatized, veneered or single chain antibodies, can also be produced. Functional fragments or portions of the foregoing antibodies include those which are reactive with the disease agent. For example, antibody fragments capable of binding to the disease agent or portion thereof, including, but not limited to scFvs, Fabs, V_{H}Hs, Fv, Fab, Fab′ and F(ab′)2 are encompassed by the invention. Such fragments can be produced by enzymatic cleavage or by recombinant techniques. For instance, papain or pepsin cleavage are used generate Fab or F(ab′)2 fragments, respectively. Antibody fragments are produced in a variety of truncated forms using antibody genes in which one or more stop codons has been introduced upstream of the natural stop site. For example, a chimeric gene encoding a F(ab′)2 heavy chain peptide portion can be designed to include DNA sequences encoding the CH1 peptide domain and hinge region of the heavy chain. Accordingly, the present invention encompasses a polynucleic acid that encodes the binding protein described herein. Binding proteins in certain embodiments are made as part of a multimeric protein, the monomer or single binding region (e.g., antibody fragments, microproteins, darpins, anticalins, adnectins, peptide mimetic molecules, aptamers, synthetic molecules, etc.) can be linked. Any combination of binding protein or binding region types can be linked. In an embodiment, the monomer or binding region of a multimeric binding protein can be linked covalently. In another embodiment, a monomer binding protein can be modified, for example, by attachment (directly or indirectly (e.g., via a linker or spacer)) to another monomer binding protein. A monomer in various embodiments is attached or genetically fused to another monomer e.g., by recombinant protein that is engineered to contain extra amino acid sequences that constitute the monomers. Thus, the DNA encoding one monomer is joined (in reading frame) with the DNA encoding the second monomer, and so on. Additional amino acids in certain embodiments are encoded between the monomers that produce an unstructured region separating the different monomers to better promote the independent folding of each monomer into its active conformation or shape. Commercially available techniques for fusing proteins are used in various embodiments to join the monomers into a multimeric binding protein of the present invention.

“Domain antibodies”, also known as “Dab”s, “Domain Antibodies”, and “dAbs” (the terms “Domain Antibodies” and “dAbs” being used as trademarks by the GlaxoSmithKline group of companies) have been described in e.g., Ward, E. S., et al.: “Binding activities of a repertoire of single immunoglobulin variable domains secreted from Escherichia coli”; Nature 341: 544-546 (1989); Holt, L. J. et al.: “Domain antibodies: proteins for therapy”; TRENDS in Biotechnology 21(11): 484-490 (2003); and WO2003/002609.

Domain antibodies essentially correspond to the V_{H} or V_{L} domains of non-camelid mammalians, in particular human 4-chain antibodies. In order to bind an epitope as a single antigen binding domain, i.e., without being paired with a V_{L} or V_{H} domain, respectively, specific selection for such antigen binding properties is required, e.g., by using libraries of human single V_{H} or V_{L} domain sequences. Domain antibodies have, like V_{H}Hs, a molecular weight of approximately 13 to approximately 16 kDa and, if derived from fully human sequences, do not require humanization for e.g., therapeutic use in humans. As in the case of V_{H}H domains, they are well expressed also in prokaryotic expression systems, providing a significant reduction in overall manufacturing cost.

Domain antibodies, as well as V_{H}H domains, can be subjected to affinity maturation by introducing one or more alterations in the amino acid sequence of one or more CDRs, which alterations result in an improved affinity of the resulting immunoglobulin single variable domain for its respective antigen, as compared to the respective parent molecule. Affinity-matured immunoglobulin single variable domain molecules of the invention may be prepared by methods known in the art, for example, as described by Marks et al., 1992, Biotechnology 10:779-783, or Barbas, et al., 1994, Proc. Nat. Acad. Sci, USA 91: 3809-3813.; Shier et al., 1995, Gene 169:147-155; Yelton et al., 1995, Immunol. 155: 1994-2004; Jackson et al., 1995, J. Immunol. 154(7):3310-9; and Hawkins et al., 1992, J. Mol. Biol. 226(3): 889 8%; K S Johnson and R E Hawkins, “Affinity maturation of antibodies using phage display”. Oxford University Press 1996.

A synthetic yeast or bacteriophage display platform can be utilized for in vitro selection of antigen-specific human-like V_{H}H, which may be used for the manufacture of therapeutics for the treatment of diseases or disorders. In this format, human-like V_{H}H genes are synthesized and cloned into a display vector adapted for use in yeast display or bacteriophage display, where they are expressed on the surface of the yeast or bacteriophage, which can then be separated based on antigen binding characteristics.

In particular instances, the libraries of the present invention may be constructed from any particular Camelid germline V_{H}H amino acid sequence by substituting amino acids beginning in framework 1 on through the end of framework 3 (including germline CDRs) with the amino acids present in the human homologue germline V_{H} amino acid sequence at the corresponding position except for the amino acids at position 44 and 45 (or positions 37, 44, 45, and 47) to produce a human-like V_{H}H germline amino acid sequence. The human-like V_{H}H germline amino acid sequence is then further modified to replace the CDRs with synthetically generated CDRs. The germline CDRs and synthetically generated CDRs may be defined using any of the currently used methods for defining CDR sequences, e.g., including but limited to Kabat, IMGT, AbM, and Chothia numbering schemes. In certain embodiments, only amino acids within the CDR are substituted. In other embodiments amino acid substitution may include an amino acid outside the CDR loop, i.e., that is the CDR area. The amino acid substitutions, both location and type, may be determined using a computer algorithm or program. Examples of substituted CDR regions for CDR1, CDR2, and CDR3 are shown in Table 2. Nucleic acid molecules are then synthesized to include each of the substitutions generated by the computer algorithm or program to produce a plurality of nucleic acid molecules, each molecule encoding one particular human-like V_{H}H.

As exemplified in Example 1, a library was designed in which the alpaca immunoglobulin heavy-chain variable (IGHV) 3S53 germline V_{H}H amino acid sequence was aligned with the human IGHV3-23*04 germline V_{H} amino acid sequence from the N-terminus to the end of framework 3. The amino acids in the alpaca V_{H}H germline sequence which differed from the amino acids at the corresponding positions in the human IGHV3-23*04 germline V_{H} amino acid sequence with the exception of the amino acids at position 44 and 45 (or positions 37, 44, 45, and 47) to produce a human-like V_{H}H germline amino acid sequence. Maintaining at least the alpaca amino acids at position 44 and 45 was sufficient to maintain stability of the human-like V_{H}H. The germline CDRs and synthetically generated CDRs for the high diversity library were defined using the IGMT numbering scheme but any numbering scheme may be used. For example, the low diversity library was constructed using the Kabat numbering scheme

Low and high diversity libraries may be constructed, which comprise the particular amino acid substitutions within the three CDR regions as shown in Table 2. The amino acid substitutions, both location and type, were determined using a computer algorithm or program. Nucleic acid molecules are then synthesized to include each of the substitutions generated by the computer algorithm or program to produce a plurality of nucleic acid molecules, each molecule encoding one particular human-like V_{H}H.

TABLE 2
CDR substitutions and particular amino acid substitutions

| Hum Low | | | |
|---|---|---|---|
| Diversity | CDR1 | CDR2 | CDR3 |
| Kabat | XYXMS | AIXSGGXTYYADSV | XXXXXXXXXXXXXXXFDX |
| | | KG | |
| IMGT | GFTFSXYX | IXSGGXT | ARXXXXXXXXXXXXXXXFDX |
| AbM | GFTFSXYXMS | AIXSGGXTY | XXXXXXXXXXXXXXXXXXXX |
| | | | XFDX |
| Chothia | GFTFSXYX | XSGGX | XXXXXXXXXXXXXXXXXXXX |
| | | | XFDX |
| Hum High | | | |
| Diversity | CDR1 | CDR2 | CDR3 |
| Kabat | XXYXMX | XISXXGXXTYYADS | XXXXXXXXXXXXXXXXXXXX |
| | | VKG | XFDX |
| IMGT | GFTFXXYAMX | XISXXGXXT | ARXXXXXXXXXXXXXXXFDX |
| AbM | GFTFXXYAMX | XISXXGXXTY | XXXXXXXXXXXXXXXXXXXX |
| | | | XFDX |
| Chothia | GFTFXXYAMX | XISXXGXX | XXXXXXXXXXXXXXXXXXXX |
| | | | XFDX |

(1) Amino acids underlined are in the CDR region but outside the CDR as defined for the particular numbering scheme.
(2) Each X is independently any amino acid except for C.
(3) For Kabat, AbM, and Chothia defined CDR3, X4-X15 may be present or absent.
(4) For IGMT defined CDR3, X6-X17 may be present or absent

### Yeast, Filamentous Fungi, and Bacterial Surface Display

More recently, target-specific V_{H}H have also been selected by bacterial (Wendel et al., Microb. Cell fact. 15:71 (2016)) or yeast (Kruse et al., Nature 504:101-106 (2013); Rychaert et al., J. Biotechnol. 15: 93-98 (2010); McMahon et al., Nat. Struct. Mol. Biol. 25:289-296 (2018) surface display followed by cell sorting. The major advantage of cell-surface display is the compatibility of these methods with the quantitative and multi-parameter analysis offered by flow cytometry. In this connection, each individual cell of the library can be investigated one by one for the display level of the cloned affinity reagent and its antigen occupancy in real time, Nat. Biotechnol. 15:553-557 (1997)), under well-controlled conditions including buffer composition, pH, temperature and antigen concentration. Accordingly, high-throughput fluorescence-activated cell sorting (FACS) allows the selection and recovery of separate cell populations, displaying binders with different predesignated properties.

Saccharomyces cerevisiae cells, displaying up to hundred thousand copies of a unique affinity reagent fused to the N-terminal end of the Aga2p subunit (Boder & Wittrup, Ibid.) are now widely used as an alternative for display methods based on filamentous phage. Uchański et al. in Sci. reps. 9:382 (2019) disclose a yeast display system wherein each V_{H}H is fused at its C-terminus to the N-terminus of Aga2p. The display level of a cloned V_{H}H on the surface of an individual yeast cell can be monitored through a covalent fluorophore that is attached in a single enzymatic step to an orthogonal acyl carrier protein (ACP) tag³⁵.

The switchable display/secretion system is another yeast display system, which is disclosed in Shaheen et al., PLoS One 8, e70190 (2013); U.S. Pat. No. 9,365,846; and, U.S. Pat. No. 10,106,598. Previous methods relied on capturing antibodies on the cell surface following secretion in culture medium. The switchable display/secretion system avoids cross-contamination between clones within the same culture by capturing the antibody prior to secretion. Advantageously, embodiments of the present invention allow co-secretion of the displayed molecule allowing further in vitro analysis. Thus, the switchable display/secretion system enables rapid characterization of lead molecules.

The switchable display/secretion system comprises a yeast or filamentous host cell comprising a nucleic acid molecule encoding bait comprising an Fc immunoglobulin domain or functional fragment thereof sufficient to for an Fc pairwise interaction fused at the C-terminus to a surface anchor polypeptide or functional fragment thereof operably linked to a regulatable promoter; and a diverse population of nucleic acid molecules encoding human-like V_{H}Hs fused to an Fc domain or functional fragment thereof, each nucleic acid molecule operably linked to a regulatable promoter (e.g., the nucleic acid molecule library disclosed herein. In particular embodiments, the regulatable promoter is selected from the group consisting of a GUT1 promoter, a GADPH promoter, a GAL promoter, or a PCK1 promoter.

Regulatory sequences which may be used in the practice of the yeast display methods disclosed herein include signal sequences, promoters, and transcription terminator sequences. It is generally preferred that the regulatory sequences used be from a species or genus that is the same as or closely related to that of the host cell or is operational in the host cell type chosen. Examples of signal sequences include those of Saccharomyces cerevisiae invertase; the Aspergillus niger amylase and glucoamylase; human serum albumin; Kluyveromyces maxianus inulinase; and Pichia pastoris mating factor and Kar2. Signal sequences shown herein to be useful in yeast and filamentous fungi include, but are not limited to, the alpha mating factor pre-sequence and pre-prosequence from Saccharomyces cerevisiae; and signal sequences from numerous other species.

Examples of promoters include promoters from numerous species, including but not limited to alcohol-regulated promoter, tetracycline-regulated promoters, steroid-regulated promoters (e.g., glucocorticoid, estrogen, ecdysone, retinoid, thyroid), metal-regulated promoters, pathogen-regulated promoters, temperature-regulated promoters, and light-regulated promoters. Specific examples of regulatable promoter systems well known in the art include but are not limited to metal-inducible promoter systems (e.g., the yeast copper-metallothionein promoter), plant herbicide safner-activated promoter systems, plant heat-inducible promoter systems, plant and mammalian steroid-inducible promoter systems, Cym repressor-promoter system (Krackeler Scientific, Inc. Albany, NY), RheoSwitch System (New England Biolabs, Beverly MA), benzoate-inducible promoter systems (See WO2004/043885), and retroviral-inducible promoter systems. Other specific regulatable promoter systems well-known in the art include the tetracycline-regulatable systems (See for example, Berens & Hillen, Eur J Biochem 270; 3109-3121 (2003)), RU 486-inducible systems, ecdysone-inducible systems, and kanamycin-regulatable system. Yeast-specific promoters include but are not limited to the Saccharomyces cerevisiae TEF-1 promoter, Pichia pastoris GAPDH promoter, Pichia pastoris GUT1 promoter, PMA-1 promoter, Pichia pastoris PCK-1 promoter, and Pichia pastoris AOX-1 and AOX-2 promoters. For temporal expression of the GPI-IgG capture moiety and the immunoglobulins, the Pichia pastoris GUT1 promoter operably linked to the nucleic acid molecule encoding the GPI-IgG capture moiety and the Pichia pastoris GAPDH promoter operably linked to the nucleic acid molecule encoding the immunoglobulin may be used. In particular embodiments, the regulatable promoter is selected from the group consisting of a GUT1 promoter, a GADPH promoter, a GAL promoter, or a PCK1 promoter.

Examples of transcription terminator sequences include transcription terminators from numerous species and proteins, including but not limited to the Saccharomyces cerevisiae cytochrome C terminator; and Pichia pastoris ALG3 and PMA1 terminators.

Host cells useful for display include Pichia pastoris, Pichia, finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuun, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum and Neurospora crassa. Various yeasts, such as K. lactis, Pichia pastoris, Pichia methanolica, and Hansenula polymorpha are particularly suitable for cell culture because they are able to grow to high cell densities and secrete large quantities of recombinant protein. Likewise, filamentous fungi, such as Aspergillus niger, Fusarium sp, Neurospora crassa and others can be used to produce glycoproteins of the invention at an industrial scale.

Host cells displaying human-like V_{H}H that bind a target of interest can be identified and isolated by incubating the host cells with the target of interest conjugated to a detectable moiety.

### 1. Generation of Binding Proteins

Binding proteins capable of binding an antigen expressed on the brain (e.g., Abeta) and methods of making the same are provided. The binding protein can be generated using various techniques. Expression vectors, host cell and methods of generating the binding protein are provided and are well known in the art.

### A. Generation of Monoclonal Antibodies

The variable domains of the binding protein can be obtained from parent antibodies, including polyclonal Abs and mAbs capable of binding antigens of interest. These antibodies may be naturally occurring or may be generated by recombinant technology. The person of ordinary skill in the art is well familiar with many methods for producing antibodies, including, but not limited to using hybridoma techniques, selected lymphocyte antibody method (SLAM), use of a phage, yeast, or RNA-protein fusion display or other library, immunizing a non-human animal comprising at least some of the human immunoglobulin locus, and preparation of chimeric, CDR-grafted, and humanised antibodies. See, e.g., US Patent Publication No. 20090311253. Variable domains may also be prepared using affinity maturation techniques.

### B. Criteria for Selecting Binding Proteins

An embodiment is provided comprising selecting binding proteins (e.g., antibodies) with at least one or more properties desired in the binding protein. In an embodiment, the desired property is one or more parameters, such as, for example, antigen specificity, affinity to antigen, potency, biological function, epitope recognition, stability, solubility, production efficiency, immunogenicity, pharmacokinetics, bioavailability, tissue cross reactivity, or orthologous antigen binding. See, e.g., US Patent Publication No. 2009031 1253.

### C. Construction of Binding Proteins

The variable domains can be obtained using recombinant DNA techniques from binding proteins generated by any one of the methods described herein. In an embodiment, the variable domain is a murine heavy or light chain variable domain. In another embodiment, the variable domain is a CDR grafted or a humanised variable heavy or light chain domain. In an embodiment, the variable domain is a human heavy or light chain variable domain. In various embodiments, the binding protein comprises a V_{H}H.

### D. Production of Binding Proteins

The binding proteins provided herein may be produced by any of a number of techniques known in the art. For example, expression from host cells, wherein expression vector(s) encoding the all or at least one portion of binding protein are/is transfected into a host cell by standard techniques. Although it is possible to express the binding proteins provided herein in either prokaryotic or eukaryotic host cells, for example binding proteins are expressed in eukaryotic cells, for example, mammalian host cells, because such eukaryotic cells (and in particular mammalian cells) are more likely than prokaryotic cells to assemble and secrete a properly folded and immunologically active binding protein.

In an exemplary system for recombinant expression of binding proteins, a recombinant expression vector encoding both the variable domain is introduced into host cells (e.g., CHO cells) by transfection, e.g., calcium phosphate-mediated transfection. Within the recombinant expression vector, the variable domain genes are each operatively linked to enhancer/promoter regulatory elements to drive high levels of transcription of the genes. The recombinant expression vector also carries a gene, which allows for selection of cells that have been transfected with the vector using for example methotrexate selection amplification. The selected transformant host cells are cultured to allow for expression of the antigen binding fragment thereof or the entire binding protein is recovered from the culture medium. Standard molecular biology techniques are used to prepare the recombinant expression vector, transfect the host cells, select for transformants, culture the host cells and recover the binding protein from the culture medium.

### II. Uses of Binding Proteins

In an embodiment, the binding proteins provided herein are capable of binding to an Abeta in vitro or in vivo (e.g., in the brain) and/or modulating the activity of Abeta targets both in vitro and in vivo. Accordingly, such binding proteins can be used to inhibit antigen activity, e.g., in a cell culture containing the antigens, in human subjects or in other mammalian subjects having the antigens with which a binding protein provided herein cross-reacts. In another embodiment, a method for reducing antigen activity in a subject suffering from a disease or disorder in which the Abeta activity is detrimental is provided. A binding protein provided herein can be administered to a human subject for therapeutic purposes.

The bispecific binding proteins are useful as therapeutic agents to simultaneously block two different targets to enhance efficacy/safety and/or increase patient coverage.

### III. Pharmaceutical Compositions

Pharmaceutical compositions comprising one or more binding proteins or antigen binding fragments thereof, either alone or in combination with prophylactic agents, therapeutic agents, and/or pharmaceutically acceptable carriers are provided. The pharmaceutical compositions comprising binding proteins or antigen binding fragments thereof provided herein are for use in, but not limited to, diagnosing, detecting, or monitoring a disorder, in preventing, treating, managing, or ameliorating a disorder or one or more symptoms thereof, and/or in research. The formulation of pharmaceutical compositions, either alone or in combination with prophylactic agents, therapeutic agents, and/or pharmaceutically acceptable carriers, is known to one skilled in the art (U.S. Patent Publication No. 2009031 1253).

Methods of administering a prophylactic or therapeutic agent provided herein include, but are not limited to, parenteral administration (e.g., intradermal, intramuscular, intraperitoneal, intravenous and subcutaneous), epidural administration, intratumoral administration, mucosal administration (e.g., intranasal and oral routes) and pulmonary administration (e.g., aerosolized compounds administered with an inhaler or nebulizer). The formulation of pharmaceutical compositions for specific routes of administration, and the materials and techniques necessary for the various methods of administration are available and known to one skilled in the art (U.S. Patent Publication No. 2009031 1253).

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. The term “dosage unit form” refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms provided herein are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of a binding protein or antigen binding fragment thereof provided herein is 0.1-100 mg/kg, for example, 1-40 mg/kg. It is to be noted that dosage values may vary with the type and severity of the condition to be alleviated is to be further understood that for any particular subject, specific dosage regimens may be adjusted over time according to the individual need and the professional judgment of the person administering or supervising the administration of the compositions, and that dosage ranges set forth herein are exemplary only and are not intended to limit the scope or practice of the claimed composition.

### IV. Combination Therapy

A binding protein or antigen binding fragment thereof provided herein also can also be administered with one or more additional medicaments or therapeutic agents useful in the treatment of various diseases, the additional agent being selected by the skilled artisan for its intended purpose. For example, the additional agent can be a therapeutic agent art recognized as being useful to treat the disease or condition being treated by the antibody provided herein. The combination can also include more than one additional agent, e.g., two or three additional agents.

The binding protein or antigen binding fragment thereof in various embodiments is administered with an agent that is a protein, a peptide, a carbohydrate, a drug, a small molecule, and a genetic material (e.g. DNA or RNA). In various embodiments, the agent is an imaging agent, a cytotoxic agent, an angiogenesis inhibitor, a kinase inhibitor, a co-stimulation molecule blocker, an adhesion molecule blocker, an anti-cytokine antibody or functional fragment thereof, methotrexate, cyclosporin, rapamycin, FK506, a detectable label or reporter, a INF antagonist, an antirheumatic, a muscle relaxant, a narcotic, a NSAID, an analgesic, an anesthetic, a sedative, a local anesthetic, a neuromuscular blacker, an antimicrobial, an anti-psoriatic, a corticosteroid, an anabolic steroid, an erythropoietin, an immunization, an immunoglobulin, an immunosuppressive, a growth hormone, a hormone replacement drug, a radiopharmaceutical, an antidepressant, an antipsychotic, a stimulant, an asthma medication, a beta agonist, an inhaled steroid, an epinephrine or analog, a cytokine, or a cytokine antagonist.

The additional agent in various embodiments is a therapeutic agent. In various embodiments, the therapeutic agent comprises budenoside, epidermal growth factor, a corticosteroid, cyclosporin, sulfasalazine, an aminosalicylate, 6-mercaptopurine, azathioprine, metronidazole, a lipoxygenase inhibitor, mesalamine, olsalazine, balsalazide, an antioxidant, a thromboxane inhibitor, an IL-1 receptor antagonist, an anti-IL-1 β mAbs, an anti-IL-6 or IL-6 receptor mAb, a growth factor, an elastase inhibitor, a pyridinyl-imidazole compound, an antibody specific against or an agonist of TNF, LT, IL-1, IL-2, IL-6, IL-7, IL-8, IL-12, IL-13, IL-15, IL-16, IL-18, IL-23, EMAP-II, GM-CSF, FGF, or PDGF, an antibody to CD2, CD3, CD4, CD8, CD-19, CD25, CD28, CD30, CD40, CD45, CD69, CD90 or a ligand thereof, methotrexate, cyclosporin, FK506, rapamycin, mycophenolate mofetil, leflunomide, an NSAID, ibuprofen, prednisolone, a phosphodiesterase inhibitor, an adenosine agonist, an antithrombotic agent, a complement inhibitor, an adrenergic agent, IRAK, NIK, IKK, p38, a MAP kinase inhibitor, an IL-1 β converting enzyme inhibitor, a TN Fa-converting enzyme inhibitor, a‘T’-cell signaling inhibitor, a metalloproteinase inhibitor, sulfasalazine, azathioprine, a 6-mercaptopurine, an angiotensin converting enzyme inhibitor, a soluble cytokine receptor, a soluble p55 TNF receptor, a soluble p75 TNF receptor, slL-1 Rl, slL-1 Rll, slL-6R, an anti-inflammatory cytokine, IL-4, IL-10, IL-11, IL-13, or TGF3.

In various embodiments of the pharmaceutical composition, the one or more additional therapeutic agents is selected from the group consisting of a tau degrader, a tau aggregation inhibitor, a tau vaccine, an α-synuclein degrader, α-synuclein aggregation inhibitor, α-synuclein vaccine a TDP-43 degrader, a TDP-43 aggregation inhibitor, an ApoE degrader, an ApoE aggregation inhibitor, an ApoE ligand binding agonist, an ApoE ligand binding antagonist, an ApoE receptor modulator, a Trem2 receptor agonist, a NRF2 activator, a NUAK1 inhibitor, a TTBK1 inhibitor, a NLRP3 inhibitor, a rRIPK1) inhibitor, a Nox2 modulator, a proteosome modulator, a TRPML1, a proteo-lipid dysfunction/aggregation modulator, a mGluR2 modulator, a α7 nicotinic acetylcholine receptor modulator, and a phosphodiesterase type 10 (PDE10) inhibitor.

Combination therapy agents include, but are not limited to, antineoplastic agents, radiotherapy, chemotherapy such as DNA alkylating agents, cisplatin, carboplatin, anti-tubulin agents, paclitaxel, docetaxel, taxol, doxorubicin, gemcitabine, gemzar, anthracyclines, adriamycin, topoisomerase inhibitors, topoisomerase II inhibitors, 5-fluorouracil (5-FU), leucovorin, irinotecan, receptor tyrosine kinase inhibitors (e.g., erlotinib, gefitinib), COX-2 inhibitors (e.g., celecoxib), kinase inhibitors, and siRNAs.

### V. Diagnostics

The disclosure herein also provides diagnostic applications including, but not limited to, diagnostic assay methods, diagnostic kits containing one or more binding proteins or antigen binding fragments thereof, and adaptation of the methods and kits for use in automated and/or semi-automated systems. The methods, kits, and adaptations provided may be employed in the detection, monitoring, and/or treatment of a disease or disorder in an individual. This is further elucidated below.

### A. Method of Assay

The present disclosure also provides a method for determining the presence, amount or concentration of an analyte, or fragment thereof, in a test sample using at least one binding protein or antigen binding fragment as described herein. Any suitable assay as is known in the art can be used in the method. Examples include, but are not limited to, immunoassays and/or methods employing mass spectrometry.

Immunoassays provided by the present disclosure may include sandwich immunoassays, radioimmunoassay (RIA), enzyme immunoassay (EIA), enzyme-linked immunosorbent assay (ELISA), competitive-inhibition immunoassays, fluorescence polarization immunoassay (FPIA), enzyme multiplied immunoassay technique (EMIT), bioluminescence resonance energy transfer (BRET), and homogenous chemiluminescent assays, among others.

The present invention includes ELISA assays incorporating the use of a binding protein or antigen binding fragment thereof disclosed herein.

For example, such a method comprises the following steps:
(a) coat a substrate (e.g., surface of a microtiter plate well, e.g., a plastic plate) with binding proteins or antigen binding fragments thereof;
(b) apply a sample to be tested for the presence of an antigen expressed on the brain (e.g., Abeta) to the substrate;
(c) wash the plate, so that unbound material in the sample is removed;
(d) apply detectably labeled binding proteins (e.g., enzyme-linked binding proteins) which are also specific to the antigen;
(e) wash the substrate, so that the unbound, labeled binding proteins are removed;
(f) if the labeled binding proteins are enzyme linked, apply a chemical which is converted by the enzyme into a fluorescent signal; and
(g) detect the presence of the labeled binding protein.

Detection of the label associated with the substrate indicates the presence of the antigen.

In a further embodiment, the labeled binding protein or antigen binding fragment thereof is labeled with peroxidase which react with ABTS (e.g., 2,2′-azino-bis(3-ethylbenzthiazoline-6-sulphonic acid)) or 3,3′,5,5′-Tetramethylbenzidine to produce a color change which is detectable. Alternatively, the labeled binding protein or antigen binding fragment thereof is labeled with a detectable radioisotope (e.g., ³H) which can be detected by scintillation counter in the presence of a scintillant.

A binding protein or antigen binding fragment thereof of the invention may be used in a Western blot or immune-protein blot procedure. Such a procedure forms part of the present invention and includes e.g.,:
(1) optionally transferring proteins from a sample to be tested for the presence of an antigen expressed on the brain (e.g., Abeta) (e.g., from a PAGE or SDS-PAGE electrophoretic separation of the proteins in the sample) onto a membrane or other solid substrate using a method known in the art (e.g., semi-dry blotting or tank blotting); contacting the membrane or other solid substrate to be tested for the presence of bound the antigen or a fragment thereof with the binding protein or antigen binding fragment thereof of the invention. Such a membrane may take the form of a nitrocellulose or vinyl-based (e.g., polyvinylidene fluoride (PVDF)) membrane to which the proteins to be tested for the presence of the antigen in a non-denaturing PAGE (polyacrylamide gel electrophoresis) gel or SDS-PAGE (sodium dodecyl sulfate polyacrylamide gel electrophoresis) gel have been transferred (e.g., following electrophoretic separation in the gel). Before contacting the membrane with the binding protein or fragment, the membrane is optionally blocked, e.g., with non-fat dry milk or the like so as to bind non-specific protein binding sites on the membrane.
(2) washing the membrane one or more times to remove unbound binding protein or fragment and other unbound substances; and
(3) detecting the bound binding protein or fragment.

Detection of the bound binding protein or fragment indicates that the antigen is present on the membrane or substrate and in the sample. Detection of the bound binding protein or fragment may be by binding the binding protein or fragment with a secondary antibody (an anti-immunoglobulin antibody) which is detectably labeled and, then, detecting the presence of the secondary antibody.

### B. Kit

A kit for assaying a test sample for the presence, amount or concentration of an analyte, or fragment thereof, in a test sample is also provided. The kit comprises at least one component for assaying the test sample for the analyte, or fragment thereof, and instructions for assaying the test sample for the analyte, or fragment thereof. The at least one component for assaying the test sample for the analyte, or fragment thereof, can include a composition comprising a binding protein or antigen binding fragment, as disclosed herein, and/or an anti-analyte binding protein (or a fragment, a variant, or a fragment of a variant thereof), which is optionally immobilized on a solid phase.

Optionally, the kit may comprise a calibrator or control, Which may comprise isolated or purified analyte. The kit can comprise at least one component for assaying the test sample for an analyte by immunoassay and/or mass spectrometry. The kit components, including the analyte, binding protein or antigen binding fragment thereof, and/or anti-analyte binding protein, or fragments thereof, may be optionally labeled using any art-known detectable label. The materials and methods for the creation provided for in the practice of the present disclosure would be known to one skilled in the art (U.S. Patent Publication No. 2009031 1253).

### C. Adaptation of Kits and Methods

The kit (or components thereof), as well as the method of determining the presence, amount or concentration of an analyze in a test sample by an assay, such as an immunoassay as described herein, can be adapted for use in a variety of automated and semi-automated systems (including those wherein the solid phase comprises a microparticle), as described, for example, in U.S. Pat. Nos. 5,089,424 and 5,006,309.

It will be readily apparent to those skilled in the art that other suitable modifications and adaptations of the methods described herein are obvious and may be made using suitable equivalents without departing from the scope of the embodiments disclosed herein. Having now described certain embodiments in detail, the same will be more clearly understood by reference to the following examples, which are included for purposes of illustration only and are not intended to be limiting.

### VI. Other Experimental Uses

A binding protein or antigen binding fragment thereof disclosed herein may be used as affinity purification agents. In this process, the binding proteins and antigen binding fragments thereof are immobilized on a solid phase such a Sephadex, glass or agarose resin or filter paper, using methods well known in the art. The immobilized binding protein or fragment is contacted with a sample containing an antigen expressed on the brain (e.g., Abeta) (or a fragment thereof) to be purified, and thereafter the support is washed with a suitable solvent that will remove substantially all the material in the sample except the antigen, which is bound to the immobilized antibody or fragment. Finally, the support is washed with a solvent which elutes the bound antigen. Such immobilized binding protein and fragments form part of the present invention.

Further provided are antigens for generating secondary antibodies which are useful for example for performing Western blots and other immunoassays discussed herein. In particular, polypeptides are disclosed which comprise the variable regions and/or CDR sequences of a binding protein disclosed herein and which may be used to generate anti-idiotypic antibodies for use in specifically detecting the presence of the antibody, e.g., in a therapeutic context.

A binding protein and antigen binding fragments thereof disclosed herein may also be used for immunohistochemistry. Such a method forms part of the present invention and comprises, e.g.,
(1) contacting a brain to be tested for the presence of Abeta with a binding protein or antigen binding fragment thereof of the invention; and
(2) detecting the binding protein or fragment.

If the binding protein or fragment itself is detectably labeled, it can be detected directly. Alternatively, the binding protein or fragment may be bound by a detectably labeled secondary antibody which is detected.

Certain binding proteins and antigen binding fragments thereof disclosed herein may also be used for in vivo imaging. Such a method may include injection of a radiolabeled binding protein or antigen binding fragment thereof into the body of a patient to be tested for the presence and/or level of Abeta followed by nuclear imaging of the body of the patient to detect the presence of the labeled binding protein or fragment.

Imaging techniques include SPECT imaging (single photon emission computed tomography) or PET imaging (positron emission tomography). Labels include e.g., iodine-123 (¹²³I) and technetium-99m (^{99m}Tc), e.g., in conjunction with SPECT imaging or ¹¹C, ¹³N, ¹⁵O or ¹⁸F, e.g., in conjunction with PET imaging or Indium-111 (See e.g., Gordon et al., (2005) International Rev. Neurobiol. 67:385-440).

### General Methods

Standard methods in molecular biology are described in Sambrook, Fritsch and Maniatis (1982 & 1989 2nd Edition, 2001 3rd Edition) Molecular Cloning, A Laboratory Manual, Cold Spring Harbor Laboratory Press, Cold Spring Harbor, NY; Sambrook and Russell (2001) Molecular Cloning, 3rd ed., Cold Spring Harbor Laboratory Press, Cold Spring Harbor, NY; Wu (1993) Recombinant DNA, Vol. 217, Academic Press, San Diego, CA. Standard methods also appear in Ausbel, et al. (2001) Current Protocols in Molecular Biology, Vols. 1-4, John Wiley and Sons. Inc. New York, NY, which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (e.g., Coligan, et al. (2000) Current Protocols in Protein Science, Vol. 1, John Wiley and Sons, Inc., New York). Chemical analysis, chemical modification, post-translational modification, production of fusion proteins, and glycosylation of proteins are described (see, e.g., Coligan, et al. (2000) Current Protocols in Protein Science, Vol. 2, John Wiley and Sons. Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, MO; pp. 45-89; Amersham Pharmacia Biotech (2001) BioDirectory, Piscataway, N.J., pp. 384-391). Production, purification, and fragmentation of polyclonal and monoclonal antibodies are described (e.g., Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, NY; Harlow and Lane, supra). Standard techniques for characterizing ligand/receptor interactions are available (see, e.g., Coligan, et al. (2001) Current Protocols in Immunology, Vol. 4, John Wiley, Inc., New York).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, NJ; Givan (2001) Flow Cytometry, 2nd ed.; Wiley-Liss, Hoboken, NJ; Shapiro (2003) Practical Flow Cytometry, John Wiley and Sons, Hoboken, NJ). Fluorescent reagents suitable for modifying nucleic acids, including nucleic acid primers and probes, polypeptides, and antibodies, for use, e.g., as diagnostic reagents, are available (e.g., Molecular Probes (2003) Catalogue, Molecular Probes. Inc., Eugene, OR; Sigma-Aldrich (2003) Catalogue, St. Louis, MO).

### Example 1. Structure- and Sequence-Based Design of Synthetic Single-Domain Antibody Libraries

This example describes the structure- and sequence-based design of synthetic single-domain antibody libraries of the present invention.

### Structure-Based Design

V_{H}H-antigen complexes available in the Protein DataBank were identified and filtered for unique V_{H}H with sub-3.5 Å resolution and protein or peptide antigen. This yielded a total of 208 complexes. The Rosetta protein modeling software was then used to measure the predicted binding energy of each complex and the binding contributions were subdivided by region, to analyze how V_{H}H typically engage their targets. This was accomplished by measuring binding energy on a per-residue basis, then dividing the contribution by residues from a given region over binding energy over the entire V_{H}H. We found that, on average, almost 60% of the total binding energy was contributed by the CDRH3 loop, with CDRH1 and CDRH2 contributing roughly equal amounts (˜15% each) to the binding energy. Surprisingly, there was a larger contribution from the framework 2 and 3 regions than expected—in fact we observed many individual cases where the binding energy was dominated by framework residues. However, to maintain stability of the molecule, we decided to leave these residues untouched in library design. Therefore, we decided to focus equally on the CDRH1 and CDRH2 loops.

When designing a synthetic library, mutations need to be added strategically to maximize possibility of antigen interaction without destabilization. Therefore, we analyzed which positions along the CDRH1 and CDRH2 tend to contribute most strongly on an energetic basis to antigen interaction, to determine which are the highest priority to diversify. We observed that the strongest interaction tended to involve residues 31 and 33 (Kabat numbering used throughout) on the CDRH1 and residues 52 and 56 on the CDRH2. We also observed that several positions very rarely contributed to antigen binding, such as residue 26 on the CDRH1 and residues 51, 55, and 57 on the CDRH2. This fits with the understanding of the role of residue 51 in contributing to the hydrophobic core of the V_{H}H (North, B., Lehmann, A. & Dunbrack, R. L. A new clustering of antibody CDR loop conformations. J. Mol. Biol. 406, 228-256 (2011)), and the highly conserved nature of residue 26(Pappas, L. et al. Rapid development of broadly influenza neutralizing antibodies through redundant mutations. Nature 516, 418-422 (2014). From this analysis we prioritized residues 31, 33, 52, and 56 as candidates for diversification.

### Sequence-Based Library Design

In addition to structural analysis, we sought to analyze properties of V_{H}H repertoires from next-generation sequencing (NGS) datasets. We expected that the amino acid profiles in the CDRH1 and CDRH2 would shed light on which residues are most frequently available for antigen interaction and which are strictly conserved. We identified two publicly available NGS datasets of V_{H}H from alpaca (Miyazaki, N. et al. Isolation and characterization of antigen-specific alpaca (Lama pacos) V_{H}H antibodies by biopanning followed by high-Throughput sequencing. J. Biochem. 158, 205-215 (2015)) and Bactrian camel (Li, X. et al. Comparative analysis of immune repertoires between bactrian Camel's conventional and heavy-chain antibodies. PLoS One 11, 1-15 (2016)), and downloaded and processed the raw data to analyze V_{H}H properties. We found that the alpaca repertoire was highly restricted in immunoglobulin heavy chain variable region gene (IGHV) and immunoglobulin heavy chain joining region gene (IGHJ) usage, with over 50% of sequences being encoded by IGHV3S53 and IGHJ4. The data were first de-deduplicated by CDRH3 before germline analysis, to exclude the possibility of a few dominant clones biasing the distribution. Since the IGHV3S53-IGHJ4 germline combination was so dominant, we chose to use this framework as the basis for the synthetic libraries. We next analyzed the CDRH1 and CDRH2 amino acid profiles in sequences encoded by IGHV3S53 and IGHJ4 (n=110,416 for alpaca, n=19,222 for camel). Although the germline gene usage was highly conserved, CDRH1 and CDRH2 amino acid sequences from alpaca and camel were highly variable. Alpaca and camel datasets shared similar patterns of conservation, with G26 on the CDRH1 and I51, G55, and T57 on CDRH2 being highly conserved. This agreed with the structural analysis which showed that these residues tended to contribute little to antigen binding. Overall, the sequence and structural data agreed on the importance of maintaining residue identity at positions critical for V_{H}H structure. Based on these two orthogonal analyses, positions 31, 33, 52, and 56 were prioritized for diversification as residues most likely to contribute to antigen recognition.

### Humanization

In addition to the alpaca IGHV3S53 framework used to construct the synthetic libraries, we designed a humanized framework that would eliminate the need for humanization after lead identification. We aligned the alpaca IGHV3S53 gene to the closest human homolog, IGHV3-23*04. There was a total of 19 amino acid differences between IGHV3S53 and IGHV3-23*04, plus one amino acid insertion in the CDRH2 of IGHV3-23*04. A previous study of V_{H}H humanization showed that two hallmark amino acids in the framework 2 (FR2) are critical for V_{H}H stability (Q44/R45), with an additional two amino acids contributing to antigen affinity but not required for stability (Y37/L47). See Vincke, C. et al. General strategy to humanize a camelid single-domain antibody and identification of a universal humanized nanobody scaffold. J. Biol. Chem. 284, 3273-3284 (2009). We therefore decided to build two humanized frameworks, one maintaining the two hallmark FR2 amino acids and one maintaining four FR2 amino acids. We refer to these two frameworks as Humanized-2AA and Humanized-4AA, respectively.

### Library Construction

Based on the previously described design principles, we designed four V_{H}H libraries for synthesis (Table 3).

TABLE 3
Final library design

| | | | CDR1 + 2 | |
|---|---|---|---|---|
| | | CDR1 + 2 | theoretical | Transformed |
| Library | Framework | diversity | diversity | library size |
| Alp_LowDiv | Alpaca | Low | 6.5 × 10⁵ | 1.2 × 10⁹ |
| Hum_LowDiv | Humanized-4AA | Low | 6.5 × 10⁵ | 1.5 × 10⁹ |
| Alp_HighDiv | Alpaca | High | 1.5 × 10¹² | 0.9 × 10⁹ |
| Hum_HighDiv | Humanized-2AA | Medium | 1.6 × 10⁷ | 1.1 × 10⁹ |
| Kruse* | Llama consensus | High | 2.3 × 10¹⁰ | 1 × 10⁹ |

Four libraries were synthesized and tested, differing in their level of CDRHI and CDRH2 diversity, and in the framework used.
*A library from McMahon, et al. that was used as a control (McMahon, C. et al. Yeast surface display platform for rapid discovery of conformationally selective nanobodies. Nat. Struct. Mol. Biol. 25, 289-296 (2018))

These synthetic libraries differed in using either fully alpaca (Alp) or partially humanized (Hum) frameworks, and in the level of diversity in the CDRH1 and CDRH2 (HighDiv for high diversity or LowDiv for low diversity). In addition to the structurally-guided low diversity libraries described above, we made two high diversity libraries randomizing the full CDRH1 and CDRH2 loops, using either degenerate codons covering a minimalist set of amino acids (Alp_HighDiv) or spiked nucleotide ratios to bias towards germline codons (Hum_HighDiv). A common CDRH3 library consisting of fragments 6-18 amino acids in length (Kabat CDRH3 definition) was spliced to each framework using overlap extension PCR (see Methods in Example 2 for details). The fully assembled V_{H}H gene fragment was then transformed into yeast and cloned into a display vector via homologous recombination. The display vector consisted of V_{H}H fused to human Fc to enable a switchable display/secretion system (Shaheen, H. H. et al. A Dual-Mode Surface Display System for the Maturation and Production of Monoclonal Antibodies in Glyco-Engineered Pichia pastoris. PLoS One 8, e70190 (2013)), with an haemagglutinin (HA) peptide tag to enable detection of V_{H}H expression on the yeast surface. The high efficiency transformation protocol was able to achieve library sizes of 10⁹ (Table 3). In addition to the four synthetic libraries designed herein, we included a synthetic library designed by McMahon, et al. 2018 derived from llama genes IGHV1S1-IGHV1S1S5 (Kruse library) to compare our synthetic libraries.

To ensure library quality, we extracted plasmid DNA from the transformed yeast and performed amplicon sequencing on the V_{H}H-encoding region. We found a distribution of CDRH3 lengths as expected. In addition, we observed that diversity was introduced correctly into the CDRH1 and CDRH2 as dictated by the design principles.

### Peptide Campaign

The next antibody discovery campaign was performed against a 40-amino acid Abeta peptide (SEQ ID NO: 1) to assess the productivity against this peptide target. Peptide binding can be challenging for V_{H}H, since peptides frequently bind in a groove formed between the heavy and light chains of a conventional antibody. See Wilson, I. A. & Stanfield, R. L. Antibody-antigen interactions: new structures and new conformational changes. Curr. Opin. Struct. Biol. 4, 857-867 (1994); Stanfield, R. L. & Wilson, I. A. Protein-peptide interactions. Curr. Opin. Struct. Biol. 5, 103-113 (1995). Investigators performed two rounds of magnetic cell sorting (MACS) and four rounds of fluorescent-activated cell sorting (FACS) selection against biotinylated test peptide. N-terminal and C-terminal biotinylated peptides were alternated during selection to avoid enriching for clones recognizing biotin-induced conformations. After four rounds of FACS selection we observed many antigen-specific binders from four of the five libraries. Library Alp_HighDiv was observed to have only reagent-specific binders after the second round of FACS and was therefore excluded from further analysis (data not shown). NGS analysis showed a clonal diversity ranging from 1.6% unique (Hum_LowDiv) to 7.3% unique (Alp_LowDiv) in the final sorted population. The CDRH3 distribution did not show a clear skewing to longer loops, in contrast to the long loops seen after mPD-1 selection.

To determine which region of the test peptide was being targeted by the libraries, we incubated different overlapping peptides with the sorted library outputs and measured binding via FACS. We used a total of six overlapping peptides spanning the length of the Abeta peptide, based on the reported binding epitopes of known mAbs against the peptide. The four libraries exhibited similar patterns of epitope recognition. The majority of clones recognized test peptide at amino acids 8-40 of SEQ ID NO: 1, with many of those also recognizing amino acids 17-40 of SEQ ID NO: 1. Libraries Hum_HighDiv and Kruse show a notable difference in binding to amino acids 8-40 vs. amino acids 17-40 of Abeta, indicating that there are clones targeting the internal region of the peptide (residues 8-17). There was very little binding observed to Abeta amino acids 1-16 in any of the libraries. Overall, investigators concluded that all libraries produce clones targeting a variety of epitopes covering residues 8-17 and 17-40 of the Abeta peptide, and that there is not significant difference between the libraries in their epitope coverage.

Investigators then produced multiple recombinant clones to characterize binding affinity using biolayer interferometry. See Table 4. We observed clear differences between the libraries in terms of their binding affinities. Library Alp_LowDiv produced clones with the weakest binding affinities, ranging from 100-400 nM. Hum_LowDiv produced a similar profile, but with two clones with affinity near 40 nM. Hum_HighDiv produced by far the best clones, with many showing sub-100 nM affinity, and one clone with an affinity of 5 nM. Although we produced seven clones from the Kruse library (see Table 5), only three of the seven produced protein, and of the three, binding affinity could only be measured for one (˜50 nM). (see Table 5). We therefore conclude that all our synthetic libraries were highly productive in generative binders against the Abeta peptide, with Hum_HighDiv producing the highest affinity clones.

### Example 2. Analysis of the V_{H}Hs that Bind Abeta

### Structural Analysis

To determine the structural variation in naturally occurring V_{H}H, we used a dataset of V_{H}H-antigen co-complexes from the Protein DataBank (PDB; rcsb.org). Annotated structures were downloaded from the Structural Antibody Database (SAbDab). Dunbar, J. et al. SAbDab: the structural antibody database. Nucleic Acids Res. 42, D1140-6 (2014). The filtered set of structures consisted of all unique V_{H}H-antigen complexes with protein or peptide antigens and a resolution of <3.5 Å. The structures were downloaded and manually processed to remove water and non-protein residues and renumbered starting from residue 1. Binding energies of the V_{H}H-antigen complexes were estimated using the Rosetta molecular modeling suite, version 3.819,41. Each complex was refined using Rosetta relax with constraints to the starting coordinates to prevent the backbone from making substantial movements. Constraints were placed on all Ca atoms with a standard deviation of 1.0 Å. Binding energy per residue was calculated using a custom RosettaScripts XML protocol (Fleishman, S. J. et al. RosettaScripts: a scripting language interface to the Rosetta macromolecular modeling suite. 6, e20161 (2011)) using the REF2015 score function 19. Position of CDR loops was defined using the IMGT/DomainGapAlign tool. See Lo, B. K. C. & Lefranc, M.-P. IMGT, The International ImMunoGeneTics Information System®. Antib. Eng. 33, 27-50 (2004). Binding energy (ΔΔG) and fractional binding energy (ΔΔGfractional) of each V_{H}H region were calculated as follows:

ΔΔGₜₒₜₐₗ=E_{complex}−E_{V}_{_{H}}_{H}−E_{Ag}

ΔΔG_{fractional}=ΔΔG_{region}/ΔΔGₜₒₜₐₗ

### Sequence Analysis

Investigators downloaded two publicly available datasets of antibody repertoires from alpaca (Lama pacos) and Bactrian camel (Camelus bactrianus) from the NCBI Sequence Read Archive44 (SRA, codes DRR01858222 and SRR354421723, respectively). We downloaded the raw FASTQ files using the fastq-dump function from the SRA toolkit (Leinonen, R., Sugawara, H. & Shumway, M. The sequence read archive. Nucleic Acids Res. 39, 2010-2012 (2011)) and assembled the paired end reads using PANDAseq (Masella, A. P., Bartram, A. K., Truszkowski, J. M., Brown, D. G. & Neufeld, J. D. PANDAseq: Paired-end assembler for illumina sequences. BMC Bioinformatics 13, 559; author reply 559-60 (2012)). Germline genes were assigned using IgBLAST46 version 1.9.0, using a custom database of Vicugna pacos genes from the IMGT reference database (Lo, B. K. C. & Lefranc, M.-P. IMGT, The International ImMunoGeneTics Information System®. Antib. Eng. 33, 27-50 (2004)). Reads were filtered by the following criteria: 1) successful V and J gene assignment, with an E value cutoff of 10-4, 2) CDRH1, 2, and 3 able to be assigned, and 3) no stop codon in translated amino acid sequence (in the case of sorted outputs). Data were deduplicated by CDRH3. Sequence profiles of CDRH1 and CDRH2 amino acids were generated using the WebLogo tool (Crooks, G. E. WebLogo: A Sequence Logo Generator. Genome Res. 14, 1188-1190 (2004)). Plots were created in Python using the Matplotlib library (Hunter, J. D. Matplotlib: A 2D graphics environment. Comput. Sci. Eng. 9, 99-104 (2007)).

### Library Design

Using structural and sequence constraints, four V_{H}H libraries were designed based on fully V_{H}H and partially humanized frameworks. Humanization was done based on alignment of the V_{H}H framework to the closest human germline IGHV gene using the IMGT reference database (Lefranc, M. P. IMGT, the international imMunoGeneTics information System. Cold Spring Harb. Protoc. 6, 595-603 (2011)). Based on structural and sequence analysis two positions in the CDRH1 and CDRH2 (four positions total) were diversified in libraries Alp_LowDiv and Hum_LowDiv. Library Alp_HighDiv was diversified in 14 positions total (seven in CDRH1 and seven in CDRH2), using a reduced codon vocabulary to incorporate the amino acids most commonly observed in the NGS datasets, on a positional basis. Library Hum_HighDiv used spiked nucleotide ratios of 79:7:7:7 to maintain a proportion of 49% germline codon. Libraries were synthesized using GeneArt DNA synthesis (Thermo Fisher Scientific).

A common CDRH3 library was designed and fused to the framework of each library. The CDRH3 fragments were synthesized using trinucleotide mutagenesis (TRIM) to control amino acid composition (see for example, Shim, BMB Reps. 48:489-494 (2015); Knappik et al., J. Mol. Biol. 296: 57-86 (2000); GeneArt of Thermo Fisher Scientific).

### Library Construction and Quality Control (QC)

To construct the four libraries, genes encoding the DNA sequence of the IGHV-gene encoded region of the antibody were synthesized (Thermo Fisher Scientific), with a 5′ region conferring a 200 bp overlap with the destination vector. The full antibody gene was assembled using a three-step PCR overlap extension. First, a 3′ recombination arm of the destination vector was amplified with an HA tag inserted directly downstream of the CDRH3 region, conferring an overlap of 410 bp with the destination vector. Next the 3′ recombination arm was fused to the CDRH3 fragments using PCR overlap extension. Lastly, the IGHV-gene encoded fragment was assembled with the CDRH3-3′ overlap fragment using PCR overlap extension. Care was taken to ensure that at least 10¹¹ molecules of library DNA fragments were included in each step of overlap extension to ensure that diversity was not lost. Fully assembled fragments were blunt end cloned into the pJET1.2 vector using the CloneJet cloning kit (ThermoFisher) and 100 clones per library were sequenced to ensure library quality before yeast transformation.

### Yeast Transformation

Yeast libraries were generated by high-efficiency transformation of a genetically modified version of the BJ5465 strain (ATCC). Cells were grown to an optical density (OD) of 1.6, spun down and washed 2× with water (or, in certain cases, 1 M sorbitol) and 1× with electroporation buffer (1 M sorbitol+1 mM CaCl₂). Cells were then incubated in pre-treatment buffer (0.1 M LiAc+2.5 mM TCEP) shaking for 30 minutes at 30° C. Next, cells were spun down and wash 3× with cold electroporation buffer. Cells were then resuspended in electroporation buffer to a final concentration of 2×10⁹ cells/mL. An amount (4 micrograms; μg) of linearized vector and 12 μg insert were added to 400 μL cells per cuvette. Electroporation using the exponential decay protocol was performed with a 2 mm cuvette with the following parameters: 2.75 kV, 200Ω resistance. 25 uF capacitance, typically resulting in a time constant of 3.5-4.0 ms. After electroporation, recovery media (equal parts YPD media and 1 M sorbitol) was added and cells were incubated shaking for 1 hour at 30° C. Cells were then spun down and resuspended in 1 M sorbitol at dilutions of 10⁻⁶, 10⁻⁷, and 10⁻⁸, and plated on glucose dropout media lacking leucine. Colonies were counted after three days growth to measure number of transformants.

### Next-Generation Sequencing (NGS) and Analysis

Library characteristics after transformation and selection were assessed by next-generation sequencing. Roughly 5×10⁸ cells were spun down from each transformed library, plasmid DNA was extracted, and the V_{H}H-encoding region was amplified by PCR. The amplified fragments were sequenced using Illumina MiSeg® 2×250 amplicon sequencing (GeneWiz). Forward and reverse reads were assembled using PANDASEQ45 and germline genes and CDR loops were assigned using IgBLAST46. Reads were filtered using the same criteria as previously described.

### Display and Induction

To induce antibody expression on the yeast surface, cells were first grown in 4% glucose dropout media lacking leucine overnight at 30° C. Cells were then switched to 4% raffinose media at a starting OD of 1.0 to derepress the GAL1 promoter and grown overnight at 30° C. The following morning, cells were switched to induction media (dropout media containing 2% raffinose and 2% galactose) to induce expression of V_{H}H under control of the GAL1 promoter. Induction media was supplemented with doxycycline at a final concentration of 22.5 μM and an O-linked glycosylation inhibitor 50 at a final concentration of 1.8 mg/L.

### MACS

To isolate antigen-specific V_{H}H, libraries underwent two rounds of magnetic sorting (MACS) followed by four rounds of fluorescence-activated cell sorting (FACS). For each library, 10¹⁰ cells from frozen transformation stocks were thawed and grown in 1 L selective media, and expression was induced as previously described. Induction level of each library before MACS was confirmed by flow cytometry. 3×10¹⁰ induced cells per library were spun down and washed 3× with PBS-F (PBS+1% bovine serum albumin). Cells were then labeled with 100 nM antigen in 20 mL PBS-F for 1 hour shaking at 30° C. After labeling, cells were spun down and washed 3× with cold PBS-F, then incubated with 500 μL streptavidin microbeads (Miltenyi Biotec) in 40 mL PBS-F for 30 minutes with rotation at 4° C. Antigen-bound cells were isolated by passing through a LS column (Miltenyi), washing 3× with 3 mL PBS-F. Cells were then eluted with 5 mL selective media and grown overnight. A subsequent round of magnetic sorting was performed, starting with 5×10⁹ induced cells per library. The second round of magnetic sorting was done following the previously described protocol, with the following modifications: 1) total volume during antigen incubation step was adjusted to 2 mL, 2) total volume during microbead incubation step was adjusted to 5 mL, and 3) anti-biotin microbeads were used to avoid enriching for streptavidin-specific binders.

### FACS

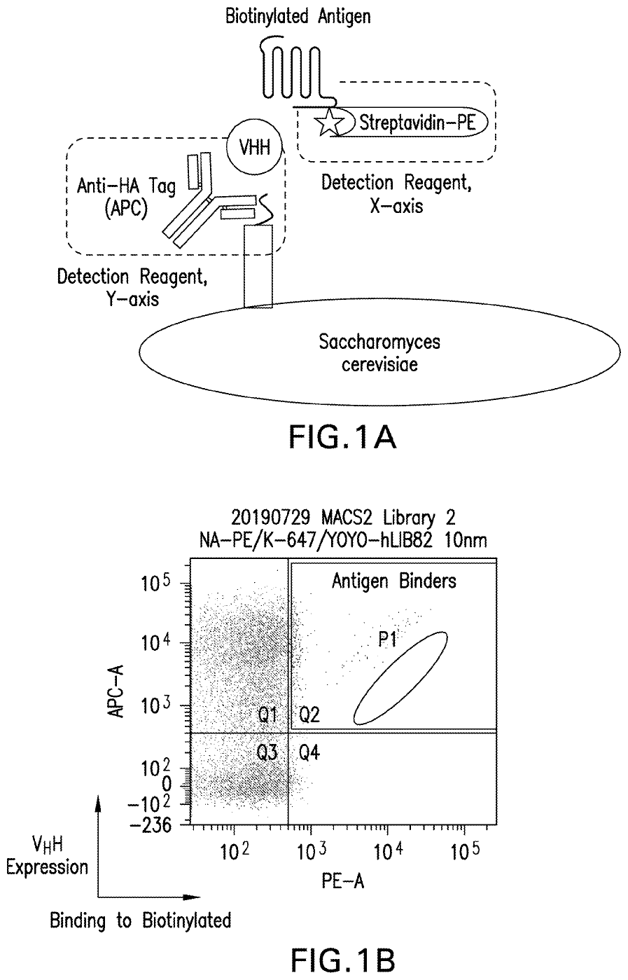

After library sizes were reduced by magnetic sorting, FACS was used to identify antigen specific V_{H}H. 5×10⁸ cells per library were passaged and induced and 10⁹ induced cells were spun down and washed 3× with PBS-F. Cells were incubated with 100 nM antigen in a total volume of 1 mL for 1 hour at 30° C. shaking, then washed again 3× with PBS-F. Next, cells were incubated with three secondary antibodies: an anti-HA tag mouse monoclonal antibody conjugated to Alexa Fluor® 647 (Thermo Fisher Scientific) to detect V_{H}H expression, neutravidin conjugated to phycoerythrin (PE; Thermo Fisher Scientific) to detect antigen binding, and YOYO1™ nuclear dye (Thermo Fisher Scientific) to measure cell viability. The secondary antibodies were added at a dilution of 1:1000, 1:200, and 1:2000, respectively, in a total volume of 10 mL, and incubated for 30 minutes on ice. After secondary incubation, cells were washed 3× with PBS-F and diluted in PBS-F for FACS screening. All FACS sorting was done on an FACSAria™ III flow cytometer (BD Biosciences). Gates were drawn to include a single population in an FSC/SSC plot and to exclude doublets on an FSC-A/FSC-H plot. In addition, the FITC-negative population was gated to remove YOYO1™-stained dead cells. For the GPCR campaign, PBS-F buffer was supplemented with detergent (0.05% dodecylmaltoside, 0.005% cholesteryl hemisuccinate) in all MACS and FACS stages. In addition, the primary incubation was performed in the presence of 20 μM antagonist. A preclear step was included in this campaign by incubating cells with 250 μL streptavidin beads at room temperature rocking for 30 minutes and passed through an LD column (Miltenyi). Flow-through cells were then subjected to FACS labeling as described above. See FIG. 1A and FIG. 1B.

Cells positive in both PE and Alexa Fluor® 647 channels were sorted into selective media, grown overnight, and passaged for a subsequent round of enrichment. The last round of selection was performed with an antigen concentration ranging from 10-50 nM to isolate high affinity binders. The secondary antibody for antigen detection was alternated between neutravidin-PE and streptavidin-DyLight™ 550 (Thermo Fisher Scientific) to reduce reagent-specific binders. In the test peptide campaign, N- and C-terminal biotin-linked test peptides were alternated during FACS rounds to reduce biotin-specific binders.

After four rounds of selection, single clones were isolated and subsequently grown and induced in a plate format. Cells were sequenced by colony polymerise chain reaction (PCR), and single clone binding in plate format was confirmed by screening against 100 nM antigen on a FACSCanto™ II flow cytometer (BD Biosciences). From each plate, clones with a unique CDRH3 sequence that displayed binding in single-cell format were selected for recombinant production.

### Recombinant Production

The V_{H}H-encoding region of selected clones was amplified and subcloned into the pTT5 mammalian expression vector, flanked by a penta-His tag. Recombinant V_{H}H were expressed by transient transfection of 30 mL cultures of ExpiCHO™-S cells (Thermo Fisher Scientific) following the recommended protocol. Supernatants were harvested after seven days and filter-sterilized with a 0.2-μm filter. Supernatant was bound to Amsphere A3 Protein A resin (JSR Life Sciences) in a batch format, with 500 μL resin per sample, and purified using a gravity column. The resin was washed with 10 column volumes (CV) PBS and eluted with 4 CV elution buffer (0.5 M glycine, pH 3.5) before the addition of 140 μL neutralization buffer (1 M Tris, pH 8) to result in a final pH of 4.8-5.0.

### Abeta Peptide Generation

An Abeta peptide (SEQ ID NO: 1 having an amino acid sequence of DAEFRHDSGYEVHHQKLVFFAEDVGSNKGATTGLMVGGVV) was synthesized by Genscript with either a N-terminal biotin or C-terminal lysine-linked biotin, at a purity of >90%. In both cases the biotin moiety was separated from the test peptide by a polyethylene glycol (PEG) 6 linker on either the N- or C-terminus, respectively. In addition, peptides spanning residues 1-16, 5-20, 8-40, 12-28, 17-40, or 25-35 were synthesized to perform epitope mapping, with a N-terminal biotin and 90% purity.

### Affinity Determination

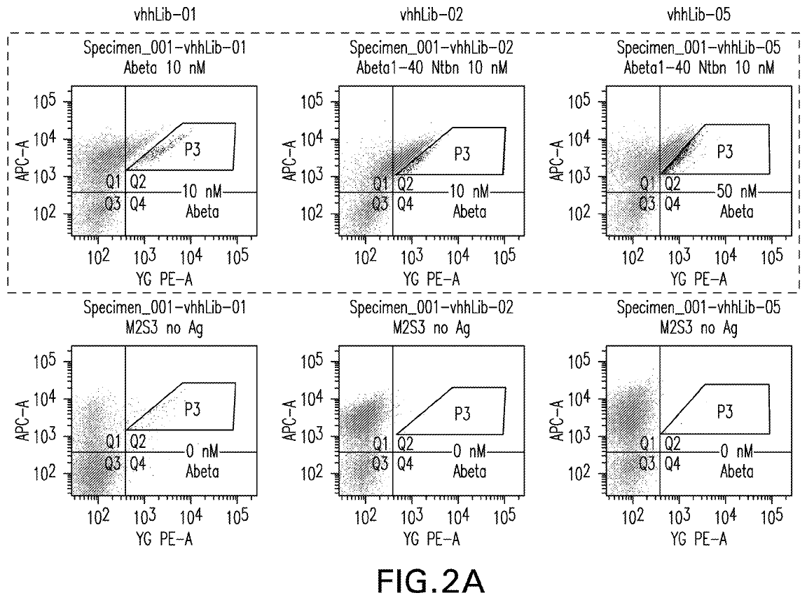

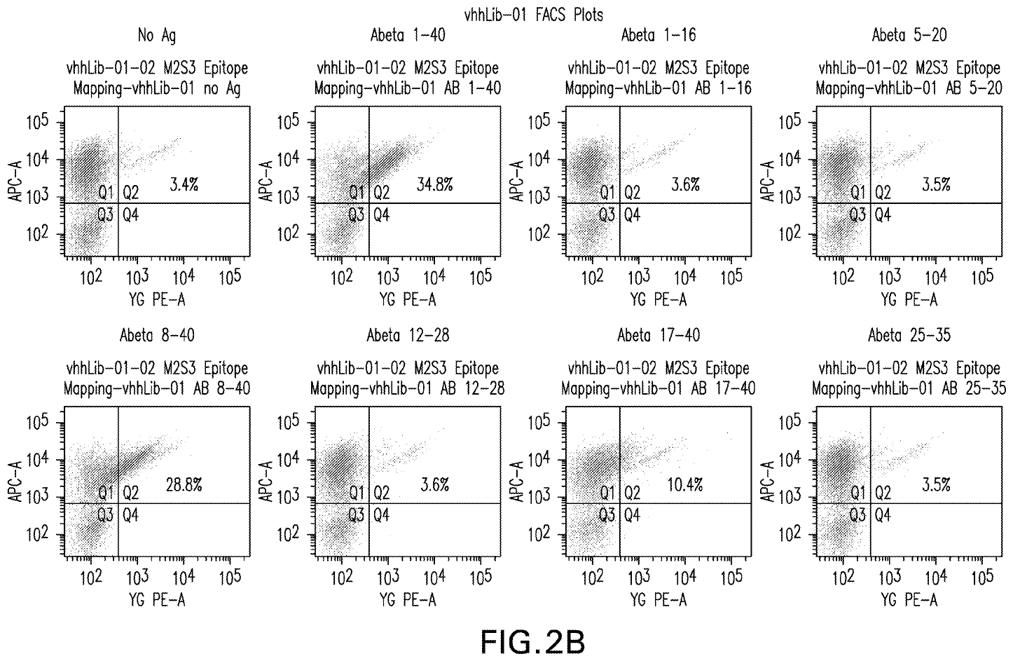

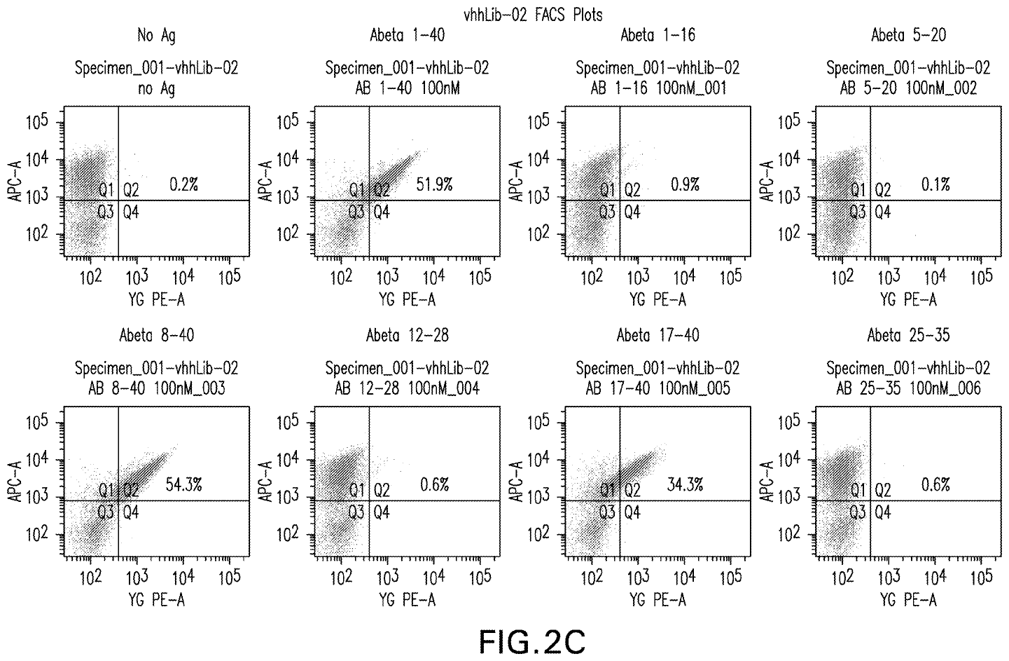

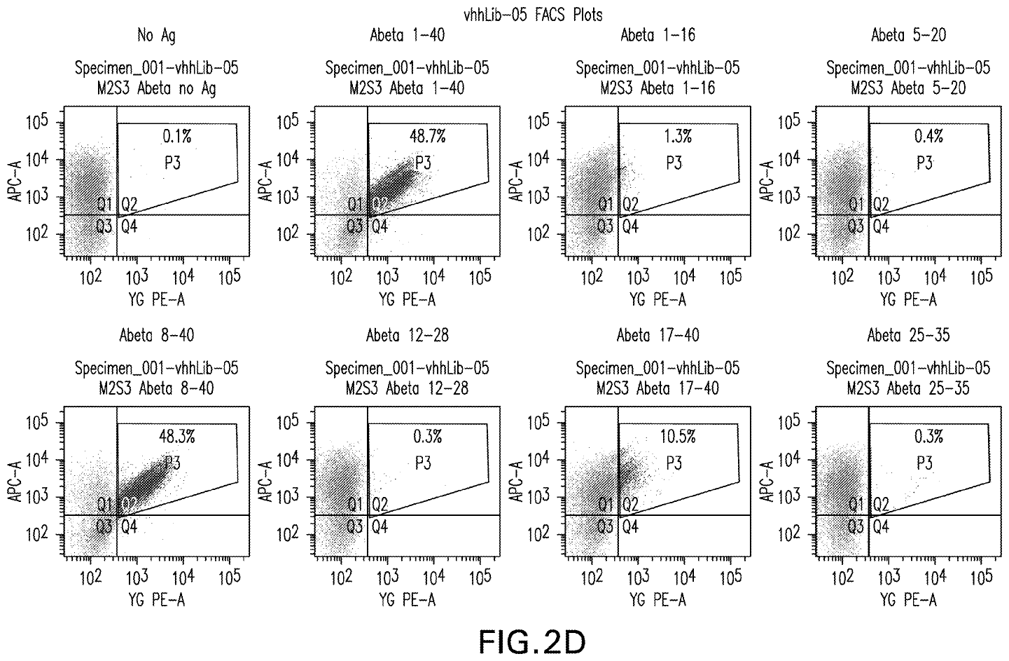

Binding affinity was measured using Biolayer Interferometry (BLI) with a ForteBio Octet HTX instrument. Biotinylated antigen was loaded onto streptavidin biosensors at a concentration of 100 nM in kinetics buffer (PBS+1% BSA). The binding experiments were performed with the following steps: 1) baseline in kinetics buffer for 30 seconds, 2) loading of antigen for 180 seconds, to achieve a loading response of at least 1 nm, 3) baseline for 60 seconds, 4) association of 1 μM V_{H}H for 300 seconds, and 5) dissociation into kinetics buffer for 180 seconds. Curves were fit to a 1:1 binding model using the ForteBio software. A negative control was included in all plates, which was un-transfected mammalian cells subjected to the same purification process, to account for the effect of any carryover protein contaminants from cell culture. The V_{H}Hs showed a range of different binding affinities. Data for the binding is shown in Table 5 and FIGS. 2A-D.

### Immunohistochemistry Staining

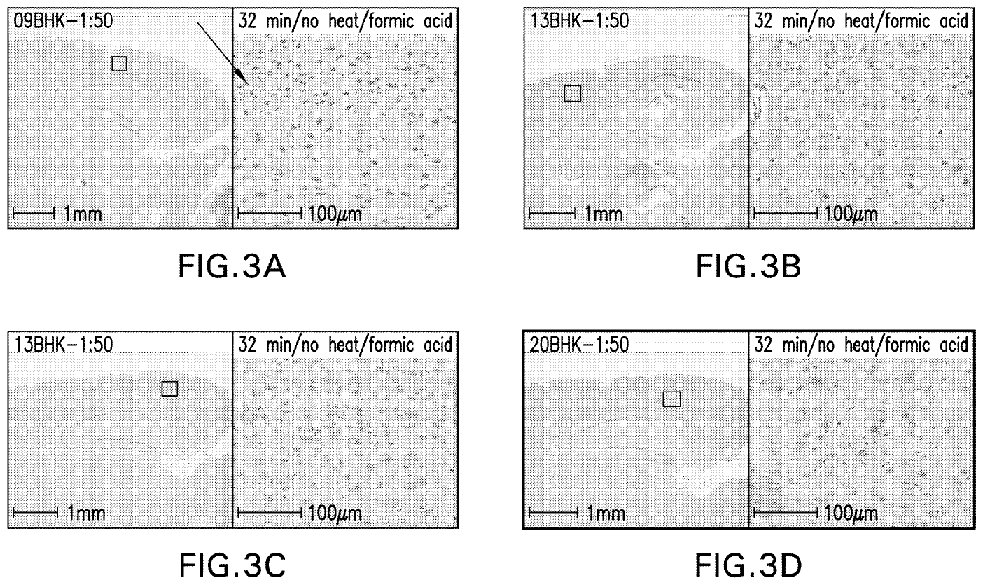

Cell staining of nonfrozen, formalin-fixed specimens (FFPE) from 4-month-old transgenic mice was performed using biotinylated V_{H}Hs described herein. The transgenic mice utilized were CRND8 (TgCRND8), which are utilized in a mouse model of Alzheimer's disease-like amyloid pathogenesis that overexpresses an amyloid precursor protein containing the Swedish and Indiana familial AD mutations (K670N/M671L and V717F). See Del Vecchio et at, vol. 367, issue 2, 2 Sep. 2004, Pages 164-167. Biotinylated V_{H}Hs were generated and incubated (at 37° C. for 32 minutes) on the FFPE (which were pre-treated with or without 70% formic acid). The biotinylated V_{H}Hs were at a concentration of 0.2-0.6 mg/ml and then diluted to 1:50 to 1:1000 prior to incubation. Data show that the V_{H}Hs bound the FFPE (FIGS. 3A-D).

### Analysis of V_{H}H Sequences

Yeast library outputs isolated from the different off-rate competition sorting gates were analyzed and the consensus sequences of the V_{H}Hs from each selection output as well as amino acids that are represented at more than 0% of the available sequences at each CDR residue positions. The consensus sequences for the V_{H}Hs and the antigen binding fragments thereof (i.e., CDR1, CDR2 and CDR3) are shown in Table 6.

TABLE 4
Amino acid sequences of the anti-Abeta VaHs and antigen binding
fragments thereof

| | SEQ ID | |
|---|---|---|
| Description | NO: | SEQUENCE |
| vhhLib01-2- | 2 | GSIFSANH |
| CDR1 | | |
| vhbLib01-2- | 3 | ISSGGLT |
| CDR2 | | |
| vhhLib01-2 | 4 | ARHQTKIYNLHYYYFDY |
| CDR3 | | |
| vhhLib01-2 | 5 | QVQLVESGGGLVQPGGSLRLSCAASGSIFSANHMGWYRQ |
| | | APGKQRELVAAISSGGLTNYADSVKGRFTISRDNAKNTVY |
| | | LQMNSLKPEDTAVYYCARHQTKIYNLHYYYFDYWGQGT |
| | | LVTVSSHHHHHH |
| vhhLib01-3- | 6 | GSIFSFNL |
| CDR1 | | |
| vhhLib01-3- | 7 | INSGGST |
| CDR2 | | |
| vhbLib01-3- | 8 | ARDYTVYVSYYDGRFDY |
| CDR3 | | |
| vhhLib01-3 | 9 | QVQLVESGGGLVQPGGSLRLSCAASGSIFSFNLMGWYRQ |
| | | APGKQRELVAAINSGGSTNYADSVKGRFTISRDNAKNTVY |
| | | LQMNSLKPEDTAVYYCARDYTVYVSYYDGRFDYWGQGT |
| | | LVTVSSHHHHHH |
| vhhLibo1-18- | 10 | GSIFSVNV |
| CDR1 | | |
| vhhLib01-18- | 11 | IDSGGYT |
| CDR2 | | |
| vhhLib01-18- | 12 | ARDYDDDDGNWEVWYGFDV |
| CDR3 | | |
| vhbLib01-18 | 13 | QVQLVESGGGLVQPGGSLRLSCAASGSIFSVNVMGWYRQ |
| | | APGKQRELVAAIDSGGYTNYADSVKGRFTISRDNAKNTV |
| | | YLQMNSLKPEDTAVYYCARDYDDDDGNWEVWYGFDVW |
| | | GQGTLVTVSSHHHHHH |
| vhhLib01-28- | 14 | GSIFSHNR |
| CDR1 | | |
| vhbLib01-28- | 15 | IGSGGYT |
| CDR2 | | |
| vhhLib01-28- | 16 | ARRTYYRWLYLYSTYFDI |
| CDR3 | | |
| vhhLib01-28 | 17 | QVQLVESGGGLVQPGGSLRLSCAASGSIFSHNRMGWYRQ |
| | | APGKQRELVAAIGSGGYTNYADSVKGRFTISRDNAKNTV |
| | | YLQMNSLKPEDTAVYYCARRTYYRWLYLYSTYFDIWGQ |
| | | GTLVTVSSHHHHHH |
| vhhLib01-34- | 18 | GSIFSVNR |
| CDR1 | | |
| vhhLib01- | 19 | IISGGAT |
| 34-CDR2 | | |
| vhhLib01- | 20 | ARRPAPNGRYHSWYAFDY |
| 34-CDR3 | | |
| vhhLib01-34 | 21 | QVQLVESGGGLVQPGGSLRLSCAASGSIFSVNRMGWYRQ |
| | | APGKQRELVAAIISGGATNYADSVKGRFTISRDNAKNTVY |
| | | LQMNSLKPEDTAVYYCARRPAPNGRYHSWYAFDYWGQG |
| | | TLVTVSSHHHHHH |
| vhhLib01-48- | 22 | GSIFSANI |
| CDR1 | | |
| vhhLib01-48- | 23 | IVSGGYT |
| CDR2 | | |
| vhhLib01-48- | 24 | ARDQDAFDHVGQYFDH |
| CDR3 | | |
| vhhLib01-48 | 25 | QVQLVESGGGLVQPGGSLRLSCAASGSIFSANIMGWYRQA |
| | | PGKQRELVAAIVSGGYTNYADSVKGRFTISRDNAKNIVYL |
| | | QMNSLKPEDTAVYYCARDQDAFDHVGQYFDHWGQGTL |
| | | VTVSSHHHHHH |
| vhhLib01-49- | 26 | GSIFSHNT |
| CDR1 | | |
| vhhLib01-49- | 27 | INSGGST |
| CDR2 | | |
| vhhLib01-49- | 28 | ARYIQTYTWGYFDY |
| CDR3 | | |
| vhbLib01-49 | 29 | QVQLVESGGGLVQPGGSLRLSCAASGSIFSHNTMGWYRQ |
| | | APGKQRELVAAINSGGSTNYADSVKGRFTISRDNAKNTVY |
| | | LQMNSLKPEDTAVYYCARYIQTYTWGYFDYWGQGTLVT |
| | | VSSHHHHHH |
| vhhLib01-50- | 30 | GSIFSYNY |
| CDR1 | | |
| vhhLib01-50- | 31 | INSGGST |
| CDR2 | | |
| vhhLib01-50- | 32 | ARLGGNGSTHYDDYFDY |
| CDR3 | | |
| vhhLib01-50 | 33 | QVQLVESGGGLVQPGGSLRLSCAASGSIFSYNYMGWYRQ |
| | | APGKQRELVAAINSGGSTNYADSVKGRFTISRDNAKNTVY |
| | | LQMNSLKPEDTAVYYCARLGGNGSTHYDDYFDYWGQGT |
| | | LVTVSSHHHHHH |
| vhhLib01-58- | 34 | GSIFSPNH |
| CDR1 | | |
| vhhLib01-58- | 35 | IYSGGTT |
| CDR2 | | |
| vhhLib01-58- | 36 | ARRTFLRGWSGYLPWFDV |
| CDR3 | | |
| vhhLib01-58 | 37 | QVQLVESGGGLVQPGGSLRLSCAASGSIFSPNHMGWYRQ |
| | | APGKQRELVAAIYSGGTTNYADSVKGRFTISRDNAKNTVY |
| | | LQMNSLKPEDTAVYYCARRTFLRGWSGYLPWFDVWGQG |
| | | TLVTVSSHHHHHH |
| vhhLib01-60- | 38 | GSIFSGND |
| CDR1 | | |
| vhhLib01-60- | 39 | IRSGGLT |
| CDR2 | | |
| vhhLib01-60- | 40 | ARDVEEWLSSIDGVWFDH |
| CDR3 | | |
| vhhLib01-60 | 41 | QVQLVESGGGLVQPGGSLRLSCAASGSIFSGNDMGWYRQ |
| | | APGKQRELVAAIRSGGLTNYADSVKGRFTISRDNAKNTVY |
| | | LQMNSLKPEDTTVYYCARDVEEWLSSIDGVWFDHWGQG |
| | | TLVTVSSHHHHHH |
| vhhLib01-75- | 42 | GSIFSSNR |
| CDR1 | | |
| vhhLib01-75- | 43 | IFSGGHT |
| CDR2 | | |
| vhhLib01-75- | 44 | ARPVDWSGPFDI |
| CDR3 | | |
| vhhLib01-75 | 45 | QVQLVESGGGLVQPGGSLRLSCAASGSIFSSNRMGWYRQ |
| | | APGKQRELVAAIFSGGHTNYADSVKGRFTISRDNAKNTVY |
| | | LQMNSLKPEDTAVYYCARPVDWSGPFDIWGQGTLVTVSS |
| | | HHHHHH |
| vhhLib01-86- | 46 | GSIFSANH |
| CDR1 | | |
| vhhLib01-86- | 47 | ILSGGVT |
| CDR2 | | |
| vhhLib01-86- | 48 | RGQVDTKYGYYYFDS |
| CDR3 | | |
| vhbLib01-86 | 49 | QVQLVESGGGLVQPGGSLRLSCAASGSIFSANHMGWYRQ |
| | | APGKQRELVAAILSGGVTNYADSVKGRFTISRDNAKNTVY |
| | | LQMNSLKPEDTAVYYCARGQVDTKYGYYYFDSWGQGTL |
| | | VTVSSTLVTVSSHHHHHH |
| vhhLib01-88- | 50 | GSIFSPNN |
| CDR1 | | |
| vhhLib01-88- | 51 | ISSGGNT |
| CDR2 | | |
| vhhLib01-88- | 52 | ARRYRQYVFLYFYSYFDV |
| CDR3 | | |
| vhhLib01-88 | 53 | QVQLVESGGGLVQPGGSLRLSCAASGSIFSPNNMGWYRQ |
| | | APGKQRELVAAISSGGNTNYADSVKGRFTISRDNAKNTVY |
| | | LQMNSLKPEDTAVYYCARRYRQYVFLYFYSYFDVWGQG |
| | | TLVTVSSHHHHHH |
| vhhLib01-89- | 54 | GSIFSDNH |
| CDR1 | | |
| vhhLib01-89- | 55 | ISSGGRT |
| CDR2 | | |
| vhbLib01-89- | 56 | ARRTYVFTTWFAFDI |
| CDR3 | | |
| vhhLib01-89 | 57 | QVQLVESGGGLVQPGGSLRLSCAASGSIFSDNHMGWYRQ |
| | | APGKQRELVAAISSGGRTNYADSVKGRFTISRDNAKNTVY |
| | | LQMNSLKPEDTAVYYCARRTYVFTTWFAFDIWGQGTLVT |
| | | VSSHHHHHH |
| vhhLib02-21- | 58 | GFTFSLYT |
| CDR1 | | |
| vhhLib02-21- | 59 | ISSGGST |
| CDR2 | | |
| vhhLib02-21- | 60 | ARYDYSWWYFDL |
| CDR3 | | |
| vhhLib02-21 | 61 | EVQLVESGGGLVQPGGSLRLSCAASGFTFSLYTMSWYRQ |
| | | APGKQRELVSAISSGGSTYYADSVKGRFTISRDNSKNTLYL |
| | | QMNSLRAEDTAVYYCARYDYSWWYFDLWGQGTLVTVS |
| | | SHHHHHH |
| vhhLib02-26- | 62 | GFTFSAYI |
| CDR1 | | |
| vhhLib02-26- | 63 | IVSGGLT |
| CDR2 | | |
| vhhLib02-26- | 64 | ARHWRWYDSNLVFDL |
| CDR3 | | |
| vhhLib02-26 | 65 | EVQLVESGGGLVQPGGSLRLSCAASGFTFSAYIMSWYRQA |
| | | PGKQRELVGAIVSGGLTYYADSVKGRFTISRDNSKNTLYL |
| | | QMNSLRAEDTAVYYCARHWRWYDSNLVFDLWGQGTLV |
| | | TVSSHHHHHH |
| vhhLib02-30- | 66 | GFTFSYYF |
| CDR1 | | |
| vhhLib02-30- | 67 | IDSGGNT |
| CDR2 | | |
| vhhLib02-30- | 68 | ARVGGLNDDVIYFDV |
| CDR3 | | |
| vhhLib02-30 | 69 | EVQLVESGGGLVQPGGSLRLSCAASGFTFSYYFMSWYRQ |
| | | APGKQRELVSAIDSGGNTYYADSVKGRFTISRDNSKNTLY |
| | | LQMNSLRAEDTAVYYCARVGGLNDDVIYFDVWGQGTLV |
| | | TVSSHHHHHH |
| vhhLib02-37- | 70 | GFTFSDYV |
| CDR1 | | |
| vhhLib02-37- | 71 | LISGEDT |
| CDR2 | | |
| vhhLib02-37- | 72 | ARYVHKSTWYYFDH |
| CDR3 | | |
| vhhLib02-37 | 73 | EVQLVESGGGLVQPGGSLRLSCAASGFTFSDYVMSWYRQ |
| | | APGKQRELVSAIISGEDTYYADSVKGRFTISRDNSKNTLYL |
| | | QMNSLRAEDTAVYYCARYVHKSTWYYFDHWGQGTLVT |
| | | VSSHHHHHH |
| vhhLib02-53- | 74 | GFTFSRYL |
| CDR1 | | |
| vhhLib02-53- | 75 | IASGGVT |
| CDR2 | | |
| vhbLib02-53- | 76 | ARGNPSERLYYYFDY |
| CDR3 | | |
| vhhLib02-53 | 77 | EVQLVESGGGLVQPGGSLRLSCAASGFTFSRYLMSWYRQ |
| | | APGKQRELVGAIASGGVTYYADSVKGRFTISRDNSKNTLY |
| | | LQMNSLRAEDTAVYYCARGNPSERLYYYFDYWGQGTLV |
| | | TVSSHHHHHH |
| vhhLib02-59- | 78 | GFTFSPYH |
| CDR1 | | |
| vhhLib02-59- | 79 | IFTGGPT |
| CDR2 | | |
| vhhLib02-59- | 80 | ARLVVDISEISGSFTFDI |
| CDR3 | | |
| vhhLib02-59 | 81 | EVQLVESGGGLVQPGGSLRLSCAASGFTFSPYHMSWYRQ |
| | | APGKQRELVSAIFTGGPTYYADSVKGRFTISRDNSKNTLYL |
| | | QMNSLHAEDTAVYYCARLVVDISEISGSFTFDIWGQGTLV |
| | | TVSSHHHHHH |
| vhhLib02-64- | 82 | GFTFSGYF |
| CDR1 | | |
| vhbLib02-64- | 83 | INSGGST |
| CDR2 | | |
| vhhLib02-64- | 84 | ARGRVATGGGYGFVHYFDV |
| CDR3 | | |
| vhhLib02-64 | 85 | EVQLVESGGGLVQPGGSLRLSCAASGFTFSGYFMSWYRQ |
| | | APGKQRELVSVINSGGSTYYADSVKGRFTISRDNSKNTLY |
| | | LQMNSLRAEDTAVYYCARGRVATGGGYGFVHYFDVWG |
| | | QGTLVTVSSHHHHHH |
| vhhLib02-91- | 86 | GFTVSDYH |
| CDR1 | | |
| vhhLib02-91- | 87 | ILSGGRT |
| CDR2 | | |
| vhhLib02-91- | 88 | ARADAIFPWFDV |
| CDR3 | | |
| vhhLib02-91 | 89 | EVQLVESGGGLVQPGGSLRLSCAASGFTVSDYHMSWYRQ |
| | | APGKQRELVSVILSGGRTYYADSVKGRFTISRDNSKNTLY |
| | | LQMNSLRAEDTAVYYCARADAIFPWFDVWGQGTLVTVSS |
| | | HHHHHH |
| vhhLib02-92- | 90 | GFTFSTYG |
| CDR1 | | |
| vhhLib02-92- | 91 | IITGEFT |
| CDR2 | | |
| vhhLib02-92- | 92 | AREETYVWDSYYDFVFDL |
| CDR3 | | |
| vhhLib02-92 | 93 | EVQLVESGGGLVQPGGSLRLSCAASGFTFSTYGMSWYRQ |
| | | APGKQRELVSANITGEFTYYADSVKGRFTISRDNSKNTLYL |
| | | QMNSLREEDTAVYYCAREETYVWDSYYDFVFDLWGQGT |
| | | LVTVSSHHHHHH |
| vhhLib02-93 | 94 | GFTFSDYH |
| CDR1 | | |
| vhhLib02-93 | 95 | INSGGYT |
| CDR2 | | |
| vhbLib02-93 | 96 | ARAQAAHYLYGYFDI |
| CDR3 | | |
| vhhLib02-93 | 97 | EVQLVESGGGLVQPGGSLRLSCAASGFTFSDYHMSWYRQ |
| | | APGKQREPVSVINSGGYTYYADSVKGRFTISRDNSKNTLY |
| | | LQMNSLRAEDTAVYYCARAQAAHYLYGYFDIWGQGTLV |
| | | TVSSHHHHHH |
| vhhLib05-5- | 98 | GFTFSSYA |
| CDR1 | | |
| vhhLib05-5- | 99 | ISGSGGST |
| CDR2 | | |
| vhhLib05-5- | 100 | ARLDYTDGINYFDY |
| CDR3 | | |
| vhhLib05-5 | 101 | EVQLLESGGGLVQPGGSLRLSCAASGFTFSSYAMSWVRQ |
| | | APGKQREWVSAISGSGGSTYYADSVKGRFTISRDNSKNTL |
| | | YLQMNSLRAEDTAVYYCARLDYTDGINYFDYWGQGTLV |
| | | TVSSTLVTVSSHHHHHH |
| vhhLib05-8- | 102 | GFTFSSYA |
| CDR1 | | |
| vhhLib05-8- | 103 | ISGSGGRT |
| CDR2 | | |
| vhhLib05-8- | 104 | ARHSIPVDGIVAFDH |
| CDR3 | | |
| vhhLib05-8 | 105 | EVQLLESGGGLVQPGGSLRLSCAASGFTFSSYAMSWVRQ |
| | | APGKQREWVSAISGSGGRTYYADSVKGRFTISRDNSKNTL |
| | | YLQMNSLRAEDTAVYYCARHSIPVDGIVAFDHWGQGTLV |
| | | TVSSTLVTVSSHHHHHH |
| vhhLib05-10- | 106 | GFTFSSYA |
| CDR1 | | |
| vhhLib05-10- | 107 | ISGSGGST |
| CDR2 | | |
| vhhLib05-10- | 108 | ARVVKVDDIVHAFDI |
| CDR3 | | |
| vhhLib05-10 | 109 | EVQLLESGGGLVQPGGSLRLSCAASGFTFSSYAMSWVRQ |
| | | APGKQREWVSAISGSGGSTYYADSVKGRFTISRDNSKNTL |
| | | YLQMNSLRAEDTAVYYCARVVKVDDIVHAFDIWGQGTL |
| | | VTVSSTLVTVSSHHHHHH |
| vhhLib05-14- | 110 | GFTFSSYA |
| CDR1 | | |
| vhhLib05-14- | 111 | ISGSGGST |
| CDR2 | | |
| vhhLib05-14- | 112 | ARLSIVDVVTVFDV |
| CDR3 | | |
| vhhLib05-14 | 113 | EVQLLESGGGLVQPGGSLRLSCAASGFTFSSYAMSWVRQ |
| | | APGKQREWVSAISGSGGSTYYADSVKGRFTISRDNSKNTL |
| | | YLQMNSLRAEDTAVYYCARLSIVDVVTVFDVWGQGTLV |
| | | TVSSTLVTVSSHHHHHH |
| vhhLib05-21- | 114 | GFTFSNYA |
| CDR1 | | |
| vhhLib05-21- | 115 | ISGNGSST |
| CDR2 | | |
| vhbLib05-21- | 116 | ARLVQLPWVPISAFDV |
| CDR3 | | |
| vhhLib05-21 | 117 | EVQLLESGGGLVQPGGSLRLSCAASGFTFSNYAMSWVRQ |
| | | APGKQREWVSVISGNGSSTYYADSVKGRFTISRDNSKNTL |
| | | YLQMNSLRAEDTAVYYCARLVQLPWVPISAFDVWGQGT |
| | | LVTVSSTLVTVSSHHHHHH |
| vhhLib05-25- | 118 | GFTFSSYA |
| CDR1 | | |
| vhhLib05-25- | 119 | ISGSGGSTYYADSVKGRFTISRDNSKNTLYLQMNSLRAED |
| CDR2 | | TAVYYCARHRQADGITYFDIWGQGTLVTVSSTLVTVSSHH |
| | | HHHH |
| vhhLib05-25- | 120 | ARHRQADGITYFDI |
| CDR3 | | |
| vhhLib05-25 | 121 | EVQLLESGGGLVQPGGSLRLSCAASGFTFSSYAMSWVRQ |
| | | APGKQREWVSAISGSGGSTYYADSVKGRFTISRDNSKNTL |
| | | YLQMNSLRAEDTAVYYCARHRQADGITYFDIWGQGTLVT |
| | | VSSTLVTVSSHHHHHH |
| vhbLib05-28- | 122 | GFTFSSYA |
| CDR1 | | |
| vhhLib05-28- | 123 | ISGSGGST |
| CDR2 | | |
| vhhLib05-28- | 124 | ARLSHNSQGTVVYNYFDI |
| CDR3 | | |
| vhhLib05-28 | 125 | EVQLLESGGGLVQPGGSLRLSCAAAGFTFSSYAMSWVRQ |
| | | APGKQREWVSAISGSGGSTYYADSVKGRFTISRDNSKNTL |
| | | YLQMNSLRAEDTAVYYCARLSHNSQGTVVYNYFDIWGQ |
| | | GTLVTVSSTLVTVSSHHHHHH |
| vhhLib05-33- | 126 | GFTFSSYA |
| CDR1 | | |
| vhhLib05-33- | 127 | ISGSGGST |
| CDR2 | | |
| vhhLib05-33- | 128 | ARLQGVQSDEVYYNYFDV |
| CDR3 | | |
| vhbLib05-33 | 129 | EVQLLESGGGLVQPGGSLRLSCAASGFTFSSYAMSWVRQ |
| | | APGKQREWVSAISGSGGSTYYADSVKGRFTISRDNSKNTL |
| | | YLQMNSLRAEDTAVYYCARLQGVQSDEVYYNYFDVWG |
| | | QGTLVTVSSTLVTVSSHHHHHH |
| vhhLib05-49- | 130 | GFTFSSYA |
| CDR1 | | |
| vhhLib05-49- | 131 | ISGSGGST |
| CDR2 | | |
| vhhLib05-49- | 132 | ARDTNQPYHGRYVYTYDFDV |
| CDR3 | | |
| vhhLib05-49 | 133 | EVQLLESGGGLVQPGGSLRLSCAASGFTFSSYAMSWVRQ |
| | | APGKQREWVSAISGSGGSTYYADSVKGRFTISRDNSKNTL |
| | | YLQMNSLRAEDTAVYYCARDINQPYHGRYVYTYDFDVW |
| | | GQGTLVTVSSTLVTVSSHHHHHH |
| vhhLib05-78- | 134 | GFTFSSYA |
| CDR1 | | |
| vhhLib05-78- | 135 | ISGSGGST |
| CDR2 | | |
| vhbLib05-78- | 136 | ARLATHVDNGLGYNYFDV |
| CDR3 | | |
| vhhLib05-78 | 137 | EVQLLESGGGLVQPGGSLRLSCAASGFTFSSYAMTWVRQ |
| | | APGKQREWVSAISGSGGSTYYADSVKGRFTISRDNSKNTL |
| | | YLQMNSLRAEDTAVYYCARLATHVDNGLGYNYFDVWG |
| | | QGTLVTVSSTLVTVSSHHHHHH |
| vhhLib05-83- | 138 | GFTFSNYA |
| CDR1 | | |
| vhhLib05-83- | 139 | ISGSGGST |
| CDR2 | | |
| vhhLib05-83- | 140 | ARLFLQQADGLYYYYAFDP |
| CDR3 | | |
| vhhLib05-83 | 141 | EVQLLESGGGLVQPGGSLRLSCAASGFTFSNYAMSWVRQ |
| | | APGKQREWVSAISGSGGSTYYADSVKGRFTISRDNSKNTL |
| | | YLQMNSLRAEDTAVYYCARLFLQQADGLYYYYAFDPWG |
| | | QGTPVTVSSTLVTVSSHHHHHH |

TABLE 5
Binding data for the Abeta VHHs

| Clone | KD (nM) | kon(1/Ms) | kdis(1/s) | Clone | KD (nM) | kon(1/Ms) | kdis(1/s) |
|---|---|---|---|---|---|---|---|
| vhhLib05-49 | 5.3 | 1.26E+04 | 6.73E−05 | vhhLib05-83 | 291.9 | 3.83E+03 | 1.12E−03 |
| vhhLib05-25 | 23.9 | 1.41E+04 | 3.37E−04 | vhbLib02-37 | 327.6 | 3.21E+03 | 1.05E−03 |
| vhhLib05-8 | 25.4 | 1.06E+04 | 2.69E−04 | vhhLib01-34 | 366.6 | 7.32E+03 | 2.68E−03 |
| vhhLib05-14 | 39.9 | 1.11E+04 | 4.43E−04 | vhhLib01-86 | 430.4 | 7.67E+03 | 3.30E−03 |
| vhhLib02-53 | 42.6 | 1.05E+04 | 4.47E−04 | vhhLib02-93 | 469.3 | 6.70E+03 | 3.14E−03 |
| vhhLib02-91 | 43.1 | 1.07E+04 | 4.60E−04 | vhhLib05-33 | 633.3 | 4.68E+03 | 2.97E−03 |

| | | | | | |
|---|---|---|---|---|---|
| vhhLibAK-46 | 56.6 | 1.12E+04 | 6.32E−04 | vhhLib01-18 | > |
| vhhLib05-21 | 103.9 | 1.07E+04 | 1.11E−03 | vhhLib01-28 | > |
| vhhLib05-28 | 113.9 | 6.37E+03 | 7.25E−04 | vhhLib01-50 | > |
| vhhLib01-49 | 116.2 | 6.32E+03 | 7.34E−04 | vhhLib02-30 | > |
| vhhLib01-48 | 152.9 | 2.18E+04 | 3.33E−03 | vhhLibAK-3 | > |
| vhhLib05-10 | 176.4 | 7.17E+03 | 1.27E−03 | vhhLibAK-36 | > |
| vhhLib01-75 | 184.8 | 1.75E+04 | 3.24E−03 | vhbLib01-60 | No protein |
| vhhLib05-5 | 189.3 | 4.66E+03 | 8.82E−04 | vhhLib01-88 | No protein |
| vhhLib02-64 | 210.2 | 1.38E+04 | 2.90E−03 | vhhLib01-89 | No protein |
| vhhLib01-2 | 212.1 | 2.98E+04 | 6.32E−03 | vhhLib02-59 | No protein |
| vhhLib05-78 | 214.3 | 6.20E+03 | 1.33E−03 | vhhLib02-92 | No protein |
| vhhLib01-3 | 239.2 | 1.48E+04 | 3.53E−03 | vhhLibAK-4 | No protein |
| vhhLib02-21 | 244.2 | 2.97E+03 | 7.24E−04 | vhhLibAK-6 | No protein |
| vhhLib02-26 | 249 | 5.50E+04 | 1.37E−02 | vhhLibAK-52 | No protein |
| vhhLib01-58 | 264.4 | 1.70E+04 | 4.50E−03 | vhhLibAK-79 | No protein |

TABLE 6
Consensus sequences for the Abeta VHHs or antigen binding fragments thereof

| | SEQ | |
|---|---|---|
| Identifier | ID NO: | Amino acid sequence |
| vhhLib-01 | 142 | QVQLVESGGGLVQPGGSLRLSCAASGSIFSINAMGWYRQAPGKQRELV |
| (Alp_LowDiv) | | AAITSGGSTNYADSVKGRFTISRDNAKNTVYLQMNSLKPEDTAVYY |
| FW | | |
| vhhLib-01 | 143 | GSIFSSNR |
| (Alp_LowDiv) | | FT AYA |
| CDR1 | | C C |
| | | D D |
| | | F F |
| | | G G |
| | | H H |
| | | I I |
| | | N N |
| | | P P |
| | | R S |
| | | T T |
| | | V V |
| | | Y Y |
| vhhLib-01 | 144 | IDSGGYT |
| (Alp_LowDiv) | | ARW A |
| CDR2 | | C C |
| | | F F |
| | | G G |
| | | H H |
| | | I I |
| | | L L |
| | | N N |
| | | P P |
| | | R R |
| | | S S |
| | | T T |
| | | V V |
| | | Y |
| vhhLib-01 | 145 | AR---------YDD--------Y-----FDV |
| (Alp_LowDiv) | | DDV TDAAAAAAAADAAAAQ YGACAI |
| CDR3 | | EQ FDDEEDDDDEDDDGY GLEP |
| | | GR GEEFFEEFEFEEEH LSGS |
| | | HT QFFGGFFGFGFFFI PVTY |
| | | L RGGHHGGHGHGGGK SYV |
| | | Q VHHIIHHKHIHHHR W |
| | | R YLIKKIILILIIIS Y |
| | | S QKLLKLNLNKKKT |
| | | V RLNPLQRNPLLLW |
| | | Y SNPQNRSQRNQN- |
| | | TPQRQSTRSPRQ |
| | | VQRSRTVSTQSR |
| | | WRSTSVWTVRTS |
| | | YSTVTYYVWSVT |
| | | TVWV WYTWV |
| | | VWYW Y VYW |
| | | WY Y W Y |
| | | Y |
| vhhLib-02 | 146 | EVQLVESGGGLVQPGGSLRLSCAASGFTFSSYAMSWYQQAPGKQRELV |
| (Hum LowDiv) | | SAITSGGSTYYADSVKGRFTISRDNSKNTLYLQMNSLRAEDTAVYY |
| FW | | |
| vhhLib-02 | 147 | GFTFSDYH |
| (Hum_LowDiv) | | L V A A |
| CDR1 | | C C |
| | | F D |
| | | G F |
| | | H G |
| | | I I |
| | | L L |
| | | P P |
| | | R R |
| | | S S |
| | | T T |
| | | V V |
| | | Y Y |
| vhhLib-02 | 148 | ILSGGRT |
| (Hum_LowDiv) | | TACAEA |
| CDR2 | | CRC D |
| | | DTD F |
| | | F V G |
| | | H H |
| | | I I |
| | | N L |
| | | S N |
| | | T P |
| | | V S |
| | | Y T |
| | | V |
| | | Y |
| vhhLib-02 | 149 | ARDDA-----------ADAY---YFDV |
| (Hum_LowDiv) | | DDA ADDAGSLGADADAADAALAA |
| CDR3 | | EFF DEFDH TDEEEDDECCVEP |
| | | GGG EFGEL VEFFFFGFDDYGY |
| | | LLQ GHHFR GGGGGHIEG T |
| | | RNV HIIGS IHHHHSLEH V |
| | | SQ ILLHT KIIIIYRGI |
| | | TR LNPIW LKKLL SHL |
| | | VS QQQLY PLLPN TIP |
| | | YT RRRN QNNQQ VLR |
| | | V TSSP RPPRR YNS |
| | | Y VTTQ SQQSS PT |
| | | YVVR TRRTT QV |
| | | WWS YSSVV RW |
| | | YYT TTYW S |
| | | V VV T |
| | | W WW V |
| | | Y YY W |
| | | Y |
| vhhLib-05 | 150 | EVQLLESGGGLVQPGGSLRLSCAASGFTFSSYAMSWVRQAPGKQREWV |
| (Hum_HighDiv) | | SXISGSGGSTYYADSVKGRFTISRDNSKNTLYLQMNSLRAEDTAVYY |
| FW | | |
| vhhLib-05 | 151 | GFTFSSYA |
| (Hum_HighDiv) | | CG |
| CDR1 | | GN |
| | | NR |
| | | RT |
| | | T |
| vhhLib-05 | 152 | AISGSGGST |
| (Hum_HighDiv) | | G ACDAG |
| CDR2 | | P RG DI |
| | | S SI RN |
| | | T VN SR |
| | | V R VT |
| | | T |
| vhhLib-05 | 153 | AR------------------------YFDV |
| (Hum_HighDiv) | | ADAADDDADAAAAAAAA DEAALTI |
| CDR3 | | DEDRGEGDGDDDDDDDD YIDD P |
| | | EGFSLGHEHFEEEEEEE LEG Y |
| | | GHG NHLGIGEGFFFFE YFH |
| | | HKI QLRHKHGHGGGGG GI |
| | | LLK RPTILIHIHHHHH HL |
| | | PNL SRVKNLILIIIII IP |
| | | QQN VSYLPNKNKLLLK KT |
| | | RRQ T NQQLPLNNNN LV |
| | | TSR V QRRNQNQQPR NW |
| | | VTS Y RSSQRPRRQS Q |
| | | YVT STTRSQSSRT R |
| | | YV TVVSTRTTSV S |
| | | W VYWTVSVVTW T |
| | | Y W YVWTWWVY V |
| | | Y WYVYYW W |
| | | Y W Y Y |
| | | Y |

Note:
In the above Table, the dash with no amino acid below can be any naturally occurring amino acid, or a gap. However, a single amino acid with a dash above it indicates that the residue at this position can either be that amino acid or it can be a gap.

### REFERENCES

1. Kaplon, H. et al. Antibodies to watch in 2020. MAbs 12, (2020).
2. Rouet, R., Dudgeon, K., Christie, M., Langley, D. & Christ, D. Fully human V_{H} single domains that rival the stability and cleft recognition of camelid antibodies. J. Biol. Chem. 290, 11905-11917 (2015).
3. To, R. et al. Isolation of monomeric human VHs by a phage selection. J. Biol. Chem. 280, 41395-41403 (2005).
4. Hamers-Casterman, C. et al. Naturally occurring antibodies devoid of light chains. Nature 363, 446-448 (1993).
5. Muyldermans, S. Nanobodies: Natural Single-Domain Antibodies. Annu. Rev. Biochem. 82, 775-797 (2013).
6. Ubah, O. C. et al. Next-generation flexible formats of VNAR domains expand the drug platform's utility and developability. Biochem. Soc. Trans. 46, 1559-1565 (2018).
7. Wesolowski, J. et al. Single domain antibodies: Promising experimental and therapeutic tools in infection and immunity. Med. Microbiol. Immunol. 198, 157-174 (2009).
8. Saerens, D., Ghassabeh, G. H. & Muyldermans, S. Single-domain antibodies as building blocks for novel therapeutics. Curt Opin. Pharmacol. 8, 600-608 (2008).
9. Vazquez-Lombardi, R. et al. Challenges and opportunities for non-antibody scaffold drugs. Drug Discov. Today 20, 1271-1283 (2015).
10. Sarker, S. A. et al. Anti-rotavirus protein reduces stool output in infants with diarrhea: A randomized placebo-controlled trial. Gastroenterology 145, 740-748.e8 (2013).
11. Laursen, N. S. et al. Universal protection against influenza infection by a multidomain antibody to influenza hemagglutinin. Science (80-.). 362, 598-602 (2018).
12. Morrison, C. Nanobody approval gives domain antibodies a boost. Nat. Rev. Drug Discov. 18, 485-487 (2019).
13. Iezzi, M. E., Policastro, L., Werbajh, S., Podhajcer, O. & Canziani, G. A. Single-domain antibodies and the promise of modular targeting in cancer imaging and treatment. Frontiers in Immunology (2018). doi:10.3389/fimmu.2018.00273
14. McMahon, C. et al. Yeast surface display platform for rapid discovery of conformationally selective nanobodies. Nat. Struct. Mol. Biol. 25, 289-296 (2018).
15. Moutel, S. et al. NaLi-H1: A universal synthetic library of humanized nanobodies providing highly functional antibodies and intrabodies. Elife 5, 1-31 (2016).
16. Zimmermann, I. et al. Synthetic single domain antibodies for the conformational trapping of membrane proteins. Elife 7, e34317 (2018).
17. Uchański, T. et al. An improved yeast surface display platform for the screening of nanobody immune libraries. Sci. Rep. 9, 1-12 (2019).
18. Shaheen, H. H. et al. A Dual-Mode Surface Display System for the Maturation and Production of Monoclonal Antibodies in Glyco-Engineered Pichia pastoris. PLoS One 8, e70190 (2013).
19. Alford, R. F. et al. The Rosetta All-Atom Energy Function for Macromolecular Modeling and Design. J. Chem. Theory Comput. 13, 3031-3048 (2017).
20. North, B., Lehmann, A. & Dunbrack, R. L. A new clustering of antibody CDR loop conformations. J. Mol. Biol. 406, 228-256 (2011).
21. Pappas, L. et al. Rapid development of broadly influenza neutralizing antibodies through redundant mutations. Nature 516, 418-422 (2014).
22. Miyazaki, N. et al. Isolation and characterization of antigen-specific alpaca (Lama pacos) VHH antibodies by biopanning followed by high-Throughput sequencing. J. Biochem. 158, 205-215 (2015).
23. Li, X. et al. Comparative analysis of immune repertoires between bactrian Camel's conventional and heavy-chain antibodies. PLoS One 11, 1-15 (2016).
24. Vincke, C. et al. General strategy to humanize a camelid single-domain antibody and identification of a universal humanized nanobody scaffold. J. Biol. Chem. 284, 3273-3284 (2009).
25. Sharpe, A. H. & Pauken, K. E. The diverse functions of the PD1 inhibitory pathway. Nat. Rev. Immunol. 18, 153-167 (2018).
26. Peters, S., Kerr, K. M. & Stahel, R. PD-1 blockade in advanced NSCLC: A focus on pembrolizumab. Cancer Treat. Rev. 62, 39-49 (2018).
27. Francisco, L. M., Sage, P. T. & Sharpe, A. H. The PD-1 pathway in tolerance and autoimmunity. Immunol. Rev. 236, 219-242 (2010).
28. Wilson, I. A. & Stanfield, R. L. Antibody-antigen interactions: new structures and new conformational changes. Curr. Opin. Struct. Biol. 4, 857-867 (1994).
29. Stanfield, R. L. & Wilson, I. A. Protein-peptide interactions. Curr. Opin. Struct. Biol. 5, 103-113(1995).
30. van Dyck, C. H. Anti-Amyloid-β Monoclonal Antibodies for Alzheimer's Disease: Pitfalls and Promise. Biol. Psychiatry 83, 311-319 (2018).
31. Mujić-Delić, A., De Wit, R. H., Verkaar, F. & Smit, M. J. GPCR-targeting nanobodies: Attractive research tools, diagnostics, and therapeutics. Trends Pharmacol. Sci. 35, 247-255 (2014).
32. Miao, Y. & McCammon, J. A. Mechanism of the G-protein mimetic nanobody binding to a muscarinic G-protein-coupled receptor. Proc. Natl. Acad. Sci. U.S.A. 115, 3036-3041 (2018).
33. Rasmussen, S. G. F. et al. Structure of a nanobody-stabilized active state of the β2adrenoceptor. Nature 469, 175-181 (2011).
34. Wingler, L. M., McMahon, C., Staus, D. P., Lefkowitz, R. J. & Kruse, A. C. Distinctive Activation Mechanism for Angiotensin Receptor Revealed by a Synthetic Nanobody. Cell 176, 479-490.e12 (2019).
35. Ahmadzadeh, V., Farajnia, S., Feizi, M. A. H. & Nejad, R. A. K. Antibody humanization methods for development of therapeutic applications. Monoclon. Antib. Immunodiagn. Immunother. 33, 67-73 (2014).
36. Hwang, W. Y. K., Almagro, J. C., Buss, T. N., Tan. P. & Foote, J. Use of human germline genes in a CDR homology-based approach to antibody humanization. Methods 36, 35-42 (2005).
37. Tan, P. et al. “Superhumanized” Antibodies: Reduction of Immunogenic Potential by Complementarity-Determining Region Grafting with Human Germline Sequences: Application to an Anti-CD28. J. Immunol. 169, 1119-1125 (2002).
38. Mader. A. & Kunert, R. Evaluation of the potency of the Anti-idiotypic antibody Ab2/3H6 mimicking gp4l as an HIV-1 vaccine in a rabbit prime/boost study. PLoS One 7, 1-8 (2012).
39. Yan, J., Li, G., Hu, Y., Ou, W. & Wan, Y. Construction of a synthetic phage-displayed Nanobody library with CDR3 regions randomized by trinucleotide cassettes for diagnostic applications. J. Transl. Med. 12, 1-12 (2014).
40. Dunbar, J. et al. SAbDab: the structural antibody database. Nucleic Acids Res. 42, D1140-6 (2014).
41. Bender, B. J. et al. Protocols for Molecular Modeling with Rosetta3 and RosettaScripts. Biochemistry 55, 4748-4763 (2016).
42. Fleishman, S. J. et al. RosettaScripts: a scripting language interface to the Rosetta macromolecular modeling suite. 6, e20161 (2011).
43. Lo, B. K. C. & Lefranc, M.-P. IMGT, The International ImMunoGeneTics Information System®. Antib. Eng. 33, 27-50 (2004).
44. Leinonen, R., Sugawara, H. & Shumway, M. The sequence read archive. Nucleic Acids Res. 39, 2010-2012 (2011).
45. Masella, A. P., Bartram, A. K., Truszkowski, J. M., Brown, D. G. & Neufeld. J. D. PANDAseq: Paired-end assembler for illumina sequences. BMC Bioinformatics 13, 559; author reply 559-60 (2012).
46. Ye, J., Ma. N., Madden, T. L. & Ostell, J. M. IgBLAST: an immunoglobulin variable domain sequence analysis tool. Nucleic Acids Res. 41, 34-40 (2013).
47. Crooks, G. E. WebLogo: A Sequence Logo Generator. Genome Res. 14, 1188-1190 (2004).
48. Hunter, J. D. Matplotlib: A 2D graphics environment. Comput. Sci. Eng. 9, 99-104 (2007).
49. Lefranc, M. P. IMGT, the international imMunoGeneTics information System. Cold Spring Harb. Protoc. 6, 595-603 (2011).
50. Argyros, R. et al. A Phenylalanine to Serine Substitution within an O-Protein Mannosyltransferase Led to Strong Resistance to PMT-Inhibitors in Pichia pastoris. PLoS One (2013). doi:10.1371/journal.pone.0062229
51. Wasilko, D. & Lee, S. E. TIPS: Titerless Infected-Cells Preservation and Scale-Up. Bioprocess. J. (2006). doi:10.12665/j53.wasilkolee

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1. A binding protein or antigen binding fragment thereof which specifically binds to Abeta comprising a variable region comprising three heavy chain complementarity determining regions (CDRs) selected from the group consisting of:
a variable region comprising CDR1, CDR2, and CDR3 comprising the amino acid sequences of SEQ ID NOs:143, 144, and 145, respectively;
(ii) a variable region comprising CDR1, CDR2, and CDR3 comprising the amino acid sequences of SEQ ID NOs:147, 148, and 149, respectively; and
(iii) a variable region comprising CDR1, CDR2, and CDR3 comprising the amino acid sequences of SEQ ID NOs:151, 152, and 153, respectively.

2-4. (canceled)

5. The binding protein or antigen binding fragment thereof of claim 1 which comprises:
(i) a CDR1 comprising an amino acid sequence selected from the group consisting of SEQ ID NOs: 2, 6, 10, 14, 18, 22, 26, 30, 34, 38, 42, 46, 50, 54, 58, 62, 66, 70, 74, 78, 82, 86, 90, 94, 98, 102, 106, 110, 114, 118, 122, 126, 130, 134, and 138;
(ii) a CDR2 comprising an amino acid sequence selected from the group consisting of SEQ ID NOs: 3, 7, 11, 15, 19, 23, 27, 31, 35, 39, 43, 47, 51, 55, 59, 63, 67, 71, 75, 79, 83, 87, 91, 95, 99, 103, 107, 111, 115, 119, 123, 127, 131, 135, and 139; or
(iii) a CDR3 comprising an amino acid sequence selected from the group consisting of SEQ ID NOs: 4, 8, 12, 16, 20, 24, 28, 32, 36, 40, 44, 48, 52, 56, 60, 64, 68, 72, 76, 80, 84, 88, 92, 96, 100, 104, 108, 112, 116, 120, 124, 128, 132, 136, and 140.

6-7. (canceled)

8. The binding protein or antigen binding fragment thereof of claim 1 comprising a variable region comprising three heavy chain CDRs selected from the group consisting of:
(a) a variable region comprising CDR1, CDR2, and CDR3 regions comprising the amino acid sequences of SEQ ID NOs: 2, 3, and 4, respectively;
(b) a variable region comprising CDR1, CDR2, and CDR3 regions comprising the amino acid sequences of SEQ ID NOs: 6, 7, and 8, respectively;
(c) a variable region comprising CDR1, CDR2, and CDR3 regions comprising the amino acid sequences of SEQ ID NOs: 10, 11, and 12, respectively;
(d) a variable region comprising CDR1, CDR2, and CDR3 regions comprising the amino acid sequences of SEQ ID NOs: 14, 15, and 16, respectively;
(e) a variable region comprising CDR1, CDR2, and CDR3 regions comprising the amino acid sequences of SEQ ID NOs: 18, 19, and 20, respectively;
(f) a variable region comprising CDR1, CDR2, and CDR3 regions comprising the amino acid sequences of SEQ ID NOs: 22, 23, and 24, respectively
(g) a variable region comprising CDR1, CDR2, and CDR3 regions comprising the amino acid sequences of SEQ ID NOs: 26, 27, and 28, respectively;
(h) a variable region comprising CDR1, CDR2, and CDR3 regions comprising the amino acid sequences of SEQ ID NOs: 30, 31, and 32, respectively;
(i) a variable region comprising CDR1, CDR2, and CDR3 regions comprising the amino acid sequences of SEQ ID NOs: 34, 35, and 36, respectively;
(j) a variable region comprising CDR1, CDR2, and CDR3 regions comprising the amino acid sequences of SEQ ID NOs: 38, 39, and 40, respectively;
(k) a variable region comprising CDR1, CDR2, and CDR3 regions comprising the amino acid sequences of SEQ ID NOs: 42, 43, and 44, respectively;
(l) a variable region comprising CDR1, CDR2, and CDR3 regions comprising the amino acid sequences of SEQ ID NOs: 46, 47, and 48, respectively;
(m) a variable region comprising CDR1, CDR2, and CDR3 regions comprising the amino acid sequences of SEQ ID NOs: 50, 51, and 52, respectively;
(n) a variable region comprising CDR1, CDR2, and CDR3 regions comprising the amino acid sequences of SEQ ID NOs: 54, 55, and 56, respectively;
(o) a variable region comprising CDR1, CDR2, and CDR3 regions comprising the amino acid sequences of SEQ ID NOs: 58, 59, and 60, respectively;
(p) a variable region comprising CDR1, CDR2, and CDR3 regions comprising the amino acid sequences of SEQ ID NOs: 62, 63, and 64, respectively;
(q) a variable region comprising CDR1, CDR2, and CDR3 regions comprising the amino acid sequences of SEQ ID NOs: 66, 67, and 68, respectively;
(r) a variable region comprising CDR1, CDR2, and CDR3 regions comprising the amino acid sequences of SEQ ID NOs: 70, 71, and 72, respectively;
(s) a variable region comprising CDR1, CDR2, and CDR3 regions comprising the amino acid sequences of SEQ ID NOs: 74, 75, and 76, respectively;
(t) a variable region comprising CDR1, CDR2, and CDR3 regions comprising the amino acid sequences of SEQ ID NOs: 78, 79, and 80, respectively;
(u) a variable region comprising CDR1, CDR2, and CDR3 regions comprising the amino acid sequences of SEQ ID NOs: 82, 83, and 84, respectively;
(v) a variable region comprising CDR1, CDR2, and CDR3 regions comprising the amino acid sequences of SEQ ID NOs: 86, 87, and 88, respectively;
(w) a variable region comprising CDR1, CDR2, and CDR3 regions comprising the amino acid sequences of SEQ ID NOs: 90, 91, and 92, respectively;
(x) a variable region comprising CDR1, CDR2, and CDR3 regions comprising the amino acid sequences of SEQ ID NOs: 94, 95, and 96, respectively;
(y) a variable region comprising CDR1, CDR2, and CDR3 regions comprising the amino acid sequences of SEQ ID NOs: 98, 99, and 100, respectively;
(z) a variable region comprising CDR1, CDR2, and CDR3 regions comprising the amino acid sequences of SEQ ID NOs: 102, 103, and 104, respectively;
(aa) a variable region comprising CDR1, CDR2, and CDR3 regions comprising the amino acid sequences of SEQ ID NOs: 106, 107, and 108, respectively;
(bb) a variable region comprising CDR1, CDR2, and CDR3 regions comprising the amino acid sequences of SEQ ID NOs: 110, 111, and 112, respectively;
(cc) a variable region comprising CDR1, CDR2, and CDR3 regions comprising the amino acid sequences of SEQ ID NOs: 114, 115, and 116, respectively;
(dd) a variable region comprising CDR1, CDR2, and CDR3 regions comprising the amino acid sequences of SEQ ID NOs: 118, 119, and 120, respectively;
(ee) a variable region comprising CDR1, CDR2, and CDR3 regions comprising the amino acid sequences of SEQ ID NOs: 122, 123, and 124, respectively;
(ff) a variable region comprising CDR1, CDR2, and CDR3 regions comprising the amino acid sequences of SEQ ID NOs: 126, 127, and 128, respectively;
(gg) a variable region comprising CDR1, CDR2, and CDR3 regions comprising the amino acid sequences of SEQ ID NOs: 130, 131, and 132, respectively;
(hh) a variable region comprising CDR1, CDR2, and CDR3 regions comprising the amino acid sequences of SEQ ID NOs: 134, 135, and 136, respectively; and
(ii) a variable region comprising CDR1, CDR2, and CDR3 regions comprising the amino acid sequences of SEQ ID NOs: 138, 139, and 140.

9. (canceled)

10. The binding protein or antigen binding fragment thereof of claim 1, which comprises a variable region sequence which is at least 85%, 90%, 95%, 98%, or 99% identical to an amino acid sequence selected from the group consisting of SEQ ID NOs: 5, 9, 13, 17, 21, 25, 29, 33, 37, 41, 45, 49, 53, 57, 61, 65, 69, 73, 77, 81, 85, 89, 93, 97, 101, 105, 109, 113, 117, 121, 125, 129, 133, 137, and 141.

11. (canceled)

12. The binding protein or antigen binding fragment thereof of claim 1 which comprises a variable region sequence which is at least 85%, 90%, 95%, 98%, or 99% identical to the amino acid sequence selected from the group consisting of SEQ ID NOs: 142, 146, and 150.

13-50. (canceled)

51. The binding protein or antigen binding fragment thereof of claim 1, wherein the binding protein or antigen binding fragment thereof binds to human Abeta or inhibits formulation of a plaque comprising Abeta.

52. (canceled)

53. The binding protein or antigen binding fragment thereof of claim 1, wherein the binding protein or antigen binding fragment thereof binds to human Abeta with a K_{D} of 50 nanomolar (nM) or less, 40 nM or less, 30 nM or less, 20 nM or less, 10 nM or less, 9 nM or less, 8 nM or less, 7 nM or less, 6 nM or less, or 5 nM or less.

54. (canceled)

55. The binding protein or antigen binding fragment thereof of claim 1, wherein the binding protein or antigen binding fragment thereof binds to Abeta in vitro or in vivo.

56. (canceled)

57. The binding protein or antigen binding fragment thereof of claim 1, wherein the binding protein or antigen binding fragment binds to soluble Abeta and/or Abeta in aggregated form.

58. The binding protein or antigen binding fragment thereof of claim 1, wherein the binding protein or antigen binding fragment binds Abeta on brain plaques.

59. The binding protein or antigen binding fragment thereof of claim 1, wherein the binding protein or antigen binding fragment thereof comprises a VHH or antigen binding fragment thereof or an antibody or antigen binding fragment thereof.

60-64. (canceled)

65. A bispecific molecule comprising the binding protein or antigen binding fragment thereof of claim 1 linked to a molecule having a second binding region.

66-67. (canceled)

68. An immunoconjugate comprising:
the binding protein or antigen binding fragment thereof of claim 1; and
a moiety selected from the group consisting of a detectable moiety, a binding moiety, a labeling moiety, or a biologically active moiety.

69. A nucleic acid comprising a nucleotide sequence that encodes the heavy chain variable region of the binding protein or antigen binding fragment thereof of claim 1.

70-71. (canceled)

72. A pharmaceutical composition comprising the binding protein or antigen binding fragment of claim 1; and a pharmaceutically acceptable carrier.

73-74. (canceled)

75. A kit comprising the binding protein or antigen binding fragment thereof of claim 1; and instructions for use.

76. A method of producing a binding protein or antigen binding fragment thereof comprising:
culturing a host cell comprising a polynucleotide encoding the amino acid sequences of any one of the binding proteins or antigen binding fragments thereof of claim 1 under conditions favorable to expression of the polynucleotide; and
optionally, recovering the binding protein or antigen binding fragment thereof from the host cell and/or culture medium.

77. A method of selectively binding Abeta on a cell, neural structure, and/or extracellular deposit comprising administering to the cell, neural structure, and/or extracellular deposit the binding protein or antigen binding fragment thereof of claim 1.

78-79. (canceled)

80. A method of treating a neurological disorder or condition associated comprising administering to a subject in need thereof a therapeutically effective amount of the binding protein or antigen binding fragment thereof of claim 1.

81. A method of inhibiting Abeta associated with a neurological disorder or condition comprising administering to a subject in need thereof a therapeutically effective amount of the binding protein or antigen binding fragment thereof of claim 1.

82-90. (canceled)


SEQUENCE LISTING (158 sequences)

SEQ ID NO: 1 - protein, 40 aa, Homo sapiens
DAEFRHDSGYEVHHQKLVFFAEDVGSNKGAIIGLMVGGVV

SEQ ID NO: 2 - protein, 8 aa, Artificial Sequence
  vhhLib01-2 - CDR1
GSIFSANH

SEQ ID NO: 3 - protein, 7 aa, Artificial Sequence
  vhhLib01-2 - CDR2
ISSGGLT

SEQ ID NO: 4 - protein, 17 aa, Artificial Sequence
  vhhLib01-2 - CDR3
ARHQTKIYNLHYYYFDY

SEQ ID NO: 5 - protein, 129 aa, Artificial Sequence
  vhhLib01-2
QVQLVESGGGLVQPGGSLRLSCAASGSIFSANHMGWYRQAPGKQRELVAAISSGGLTNYADSVKGRFTISRDNAKNTVYLQMNSLKPEDTAVYYCARHQTKIYNLHYYYFDYWGQGTLVTVSSHHHHHH

SEQ ID NO: 6 - protein, 8 aa, Artificial Sequence
  vhhLib01-3- CDR1
GSIFSFNL

SEQ ID NO: 7 - protein, 7 aa, Artificial Sequence
  vhhLib01-3- CDR2
INSGGST

SEQ ID NO: 8 - protein, 17 aa, Artificial Sequence
  vhhLib01-3- CDR3
ARDYTVYVSYYDGRFDY

SEQ ID NO: 9 - protein, 129 aa, Artificial Sequence
  vhhLib01-3
QVQLVESGGGLVQPGGSLRLSCAASGSIFSFNLMGWYRQAPGKQRELVAAINSGGSTNYADSVKGRFTISRDNAKNTVYLQMNSLKPEDTAVYYCARDYTVYVSYYDGRFDYWGQGTLVTVSSHHHHHH

SEQ ID NO: 10 - protein, 8 aa, Artificial Sequence
  GSIFSVNV
GSIFSVNV

SEQ ID NO: 11 - protein, 7 aa, Artificial Sequence
  vhhLib01-18 - CDR2 vhhLib01-18 - CDR2
IDSGGYT

SEQ ID NO: 12 - protein, 19 aa, Artificial Sequence
  vhhLib01-18-CDR3
ARDYDDDDGNWEVWYGFDV

SEQ ID NO: 13 - protein, 131 aa, Artificial Sequence
  vhhLib01-18
QVQLVESGGGLVQPGGSLRLSCAASGSIFSVNVMGWYRQAPGKQRELVAAIDSGGYTNYADSVKGRFTISRDNAKNTVYLQMNSLKPEDTAVYYCARDYDDDDGNWEVWYGFDVWGQGTLVTVSSHHHHHH

SEQ ID NO: 14 - protein, 8 aa, Artificial Sequence
  vhhLib01-28 - CDR1
GSIFSHNR

SEQ ID NO: 15 - protein, 7 aa, Artificial Sequence
  vhhLib01-28 - CDR2
IGSGGYT

SEQ ID NO: 16 - protein, 18 aa, Artificial Sequence
  vhhLib01-28-CDR3
ARRTYYRWLYLYSTYFDI

SEQ ID NO: 17 - protein, 130 aa, Artificial Sequence
  vhhLib01-28
QVQLVESGGGLVQPGGSLRLSCAASGSIFSHNRMGWYRQAPGKQRELVAAIGSGGYTNYADSVKGRFTISRDNAKNTVYLQMNSLKPEDTAVYYCARRTYYRWLYLYSTYFDIWGQGTLVTVSSHHHHHH

SEQ ID NO: 18 - protein, 8 aa, Artificial Sequence
  vhhLib01-34 - CDR1
GSIFSVNR

SEQ ID NO: 19 - protein, 7 aa, Artificial Sequence
  vhhLib01-34-CDR2
IISGGAT

SEQ ID NO: 20 - protein, 18 aa, Artificial Sequence
  vhhLib01-34-CDR3
ARRPAPNGRYHSWYAFDY

SEQ ID NO: 21 - protein, 130 aa, Artificial Sequence
  vhhLib01-34
QVQLVESGGGLVQPGGSLRLSCAASGSIFSVNRMGWYRQAPGKQRELVAAIISGGATNYADSVKGRFTISRDNAKNTVYLQMNSLKPEDTAVYYCARRPAPNGRYHSWYAFDYWGQGTLVTVSSHHHHHH

SEQ ID NO: 22 - protein, 8 aa, Artificial Sequence
  vhhLib01-48-CDR1
GSIFSANI

SEQ ID NO: 23 - protein, 7 aa, Artificial Sequence
  vhhLib01-48-CDR2
IVSGGYT

SEQ ID NO: 24 - protein, 16 aa, Artificial Sequence
  vhhLib01-48-CDR3
ARDQDAFDHVGQYFDH

SEQ ID NO: 25 - protein, 128 aa, Artificial Sequence
  vhhLib01-48
QVQLVESGGGLVQPGGSLRLSCAASGSIFSANIMGWYRQAPGKQRELVAAIVSGGYTNYADSVKGRFTISRDNAKNTVYLQMNSLKPEDTAVYYCARDQDAFDHVGQYFDHWGQGTLVTVSSHHHHHH

SEQ ID NO: 26 - protein, 8 aa, Artificial Sequence
  vhhLib01-49-CDR1
GSIFSHNT

SEQ ID NO: 27 - protein, 7 aa, Artificial Sequence
  vhhLib01-49-CDR2
INSGGST

SEQ ID NO: 28 - protein, 14 aa, Artificial Sequence
  vhhLib01-49-CDR3
ARYIQTYTWGYFDY

SEQ ID NO: 29 - protein, 126 aa, Artificial Sequence
  vhhLib01-49
QVQLVESGGGLVQPGGSLRLSCAASGSIFSHNTMGWYRQAPGKQRELVAAINSGGSTNYADSVKGRFTISRDNAKNTVYLQMNSLKPEDTAVYYCARYIQTYTWGYFDYWGQGTLVTVSSHHHHHH

SEQ ID NO: 30 - protein, 8 aa, Artificial Sequence
  vhhLib01-50-CDR1
GSIFSYNY

SEQ ID NO: 31 - protein, 7 aa, Artificial Sequence
  vhhLib01-50-CDR1
INSGGST

SEQ ID NO: 32 - protein, 17 aa, Artificial Sequence
  vhhLib01-50-CDR3
ARLGGNGSTHYDDYFDY

SEQ ID NO: 33 - protein, 129 aa, Artificial Sequence
  vhhLib01-50
QVQLVESGGGLVQPGGSLRLSCAASGSIFSYNYMGWYRQAPGKQRELVAAINSGGSTNYADSVKGRFTISRDNAKNTVYLQMNSLKPEDTAVYYCARLGGNGSTHYDDYFDYWGQGTLVTVSSHHHHHH

SEQ ID NO: 34 - protein, 8 aa, Artificial Sequence
  vhhLib01-58-CDR1
GSIFSPNH

SEQ ID NO: 35 - protein, 7 aa, Artificial Sequence
  vhhLib01-58-CDR2
IYSGGTT

SEQ ID NO: 36 - protein, 18 aa, Artificial Sequence
  vhhLib01-58-CDR3
ARRTFLRGWSGYLPWFDV

SEQ ID NO: 37 - protein, 130 aa, Artificial Sequence
  vhhLib01-58
QVQLVESGGGLVQPGGSLRLSCAASGSIFSPNHMGWYRQAPGKQRELVAAIYSGGTTNYADSVKGRFTISRDNAKNTVYLQMNSLKPEDTAVYYCARRTFLRGWSGYLPWFDVWGQGTLVTVSSHHHHHH

SEQ ID NO: 38 - protein, 8 aa, Artificial Sequence
  vhhLib01-60-CDR1
GSIFSGND

SEQ ID NO: 39 - protein, 7 aa, Artificial Sequence
  vhhLib01-60-CDR2
IRSGGLT

SEQ ID NO: 40 - protein, 18 aa, Artificial Sequence
  vhhLib01-60-CDR3
ARDVEEWLSSIDGVWFDH

SEQ ID NO: 41 - protein, 130 aa, Artificial Sequence
  vhhLib01-60
QVQLVESGGGLVQPGGSLRLSCAASGSIFSGNDMGWYRQAPGKQRELVAAIRSGGLTNYADSVKGRFTISRDNAKNTVYLQMNSLKPEDTTVYYCARDVEEWLSSIDGVWFDHWGQGTLVTVSSHHHHHH

SEQ ID NO: 42 - protein, 8 aa, Artificial Sequence
  vhhLib01-75-CDR1
GSIFSSNR

SEQ ID NO: 43 - protein, 7 aa, Artificial Sequence
  vhhLib01-75-CDR2
IFSGGHT

SEQ ID NO: 44 - protein, 12 aa, Artificial Sequence
  vhhLib01-75
ARPVDWSGPFDI

SEQ ID NO: 45 - protein, 124 aa, Artificial Sequence
  vhhLib01-75
QVQLVESGGGLVQPGGSLRLSCAASGSIFSSNRMGWYRQAPGKQRELVAAIFSGGHTNYADSVKGRFTISRDNAKNTVYLQMNSLKPEDTAVYYCARPVDWSGPFDIWGQGTLVTVSSHHHHHH

SEQ ID NO: 46 - protein, 8 aa, Artificial Sequence
  vhhLib01-86-CDR1
GSIFSANH

SEQ ID NO: 47 - protein, 7 aa, Artificial Sequence
  vhhLib01-86-CDR2
ILSGGVT

SEQ ID NO: 48 - protein, 15 aa, Artificial Sequence
  vhhLib01-86-CDR3
RGQVDTKYGYYYFDS

SEQ ID NO: 49 - protein, 135 aa, Artificial Sequence
  vhhLib01-86
QVQLVESGGGLVQPGGSLRLSCAASGSIFSANHMGWYRQAPGKQRELVAAILSGGVTNYADSVKGRFTISRDNAKNTVYLQMNSLKPEDTAVYYCARGQVDTKYGYYYFDSWGQGTLVTVSSTLVTVSSHHHHHH

SEQ ID NO: 50 - protein, 8 aa, Artificial Sequence
  vhhLib01-88-CDR1
GSIFSPNN

SEQ ID NO: 51 - protein, 7 aa, Artificial Sequence
  vhhLib01-88-CDR2
ISSGGNT

SEQ ID NO: 52 - protein, 18 aa, Artificial Sequence
  vhhLib01-88-CDR3
ARRYRQYVFLYFYSYFDV

SEQ ID NO: 53 - protein, 130 aa, Artificial Sequence
  vhhLib01-88
QVQLVESGGGLVQPGGSLRLSCAASGSIFSPNNMGWYRQAPGKQRELVAAISSGGNTNYADSVKGRFTISRDNAKNTVYLQMNSLKPEDTAVYYCARRYRQYVFLYFYSYFDVWGQGTLVTVSSHHHHHH

SEQ ID NO: 54 - protein, 8 aa, Artificial Sequence
  vhhLib01-89-CDR1
GSIFSDNH

SEQ ID NO: 55 - protein, 7 aa, Artificial Sequence
  vhhLib01-89-CDR2
ISSGGRT

SEQ ID NO: 56 - protein, 15 aa, Artificial Sequence
  vhhLib01-89-CDR3
ARRTYVFTTWFAFDI

SEQ ID NO: 57 - protein, 127 aa, Artificial Sequence
  vhhLib01-89
QVQLVESGGGLVQPGGSLRLSCAASGSIFSDNHMGWYRQAPGKQRELVAAISSGGRTNYADSVKGRFTISRDNAKNTVYLQMNSLKPEDTAVYYCARRTYVFTTWFAFDIWGQGTLVTVSSHHHHHH

SEQ ID NO: 58 - protein, 8 aa, Artificial Sequence
  vhhLib02-21-CDR1
GFTFSLYT

SEQ ID NO: 59 - protein, 7 aa, Artificial Sequence
  vhhLib02-21-CDR2
ISSGGST

SEQ ID NO: 60 - protein, 12 aa, Artificial Sequence
  vhhLib02-21-CDR3
ARYDYSWWYFDL

SEQ ID NO: 61 - protein, 124 aa, Artificial Sequence
  vhhLib02-21
EVQLVESGGGLVQPGGSLRLSCAASGFTFSLYTMSWYRQAPGKQRELVSAISSGGSTYYADSVKGRFTISRDNSKNTLYLQMNSLRAEDTAVYYCARYDYSWWYFDLWGQGTLVTVSSHHHHHH

SEQ ID NO: 62 - protein, 8 aa, Artificial Sequence
  vhhLib02-26-CDR1
GFTFSAYI

SEQ ID NO: 63 - protein, 7 aa, Artificial Sequence
  vhhLib02-26-CDR2
IVSGGLT

SEQ ID NO: 64 - protein, 15 aa, Artificial Sequence
  vhhLib02-26-CDR3
ARHWRWYDSNLVFDL

SEQ ID NO: 65 - protein, 127 aa, Artificial Sequence
  vhhLib02-26
EVQLVESGGGLVQPGGSLRLSCAASGFTFSAYIMSWYRQAPGKQRELVGAIVSGGLTYYADSVKGRFTISRDNSKNTLYLQMNSLRAEDTAVYYCARHWRWYDSNLVFDLWGQGTLVTVSSHHHHHH

SEQ ID NO: 66 - protein, 8 aa, Artificial Sequence
  vhhLib02-30-CDR1
GFTFSYYF

SEQ ID NO: 67 - protein, 7 aa, Artificial Sequence
  vhhLib02-30-CDR2
IDSGGNT

SEQ ID NO: 68 - protein, 15 aa, Artificial Sequence
  vhhLib02-30-CDR3
ARVGGLNDDVIYFDV

SEQ ID NO: 69 - protein, 127 aa, Artificial Sequence
  vhhLib02-30
EVQLVESGGGLVQPGGSLRLSCAASGFTFSYYFMSWYRQAPGKQRELVSAIDSGGNTYYADSVKGRFTISRDNSKNTLYLQMNSLRAEDTAVYYCARVGGLNDDVIYFDVWGQGTLVTVSSHHHHHH

SEQ ID NO: 70 - protein, 8 aa, Artificial Sequence
  vhhLib02-37-CDR1
GFTFSDYV

SEQ ID NO: 71 - protein, 7 aa, Artificial Sequence
  vhhLib02-37-CDR2
IISGEDT

SEQ ID NO: 72 - protein, 14 aa, Artificial Sequence
  vhhLib02-37-CDR3
ARYVHKSTWYYFDH

SEQ ID NO: 73 - protein, 126 aa, Artificial Sequence
  vhhLib02-37
EVQLVESGGGLVQPGGSLRLSCAASGFTFSDYVMSWYRQAPGKQRELVSAIISGEDTYYADSVKGRFTISRDNSKNTLYLQMNSLRAEDTAVYYCARYVHKSTWYYFDHWGQGTLVTVSSHHHHHH

SEQ ID NO: 74 - protein, 8 aa, Artificial Sequence
  vhhLib02-53-CDR1
GFTFSRYL

SEQ ID NO: 75 - protein, 7 aa, Artificial Sequence
  vhhLib02-53-CDR2
IASGGVT

SEQ ID NO: 76 - protein, 15 aa, Artificial Sequence
  vhhLib02-53-CDR3
ARGNPSERLYYYFDY

SEQ ID NO: 77 - protein, 127 aa, Artificial Sequence
  vhhLib02-53
EVQLVESGGGLVQPGGSLRLSCAASGFTFSRYLMSWYRQAPGKQRELVGAIASGGVTYYADSVKGRFTISRDNSKNTLYLQMNSLRAEDTAVYYCARGNPSERLYYYFDYWGQGTLVTVSSHHHHHH

SEQ ID NO: 78 - protein, 8 aa, Artificial Sequence
  vhhLib02-59-CDR1
GFTFSPYH

SEQ ID NO: 79 - protein, 7 aa, Artificial Sequence
  vhhLib02-59-CDR2
IFTGGPT

SEQ ID NO: 80 - protein, 18 aa, Artificial Sequence
  vhhLib02-59-CDR3
ARLVVDISEISGSFTFDI

SEQ ID NO: 81 - protein, 130 aa, Artificial Sequence
  vhhLib02-59
EVQLVESGGGLVQPGGSLRLSCAASGFTFSPYHMSWYRQAPGKQRELVSAIFTGGPTYYADSVKGRFTISRDNSKNTLYLQMNSLHAEDTAVYYCARLVVDISEISGSFTFDIWGQGTLVTVSSHHHHHH

SEQ ID NO: 82 - protein, 8 aa, Artificial Sequence
  vhhLib02-64-CDR1
GFTFSGYF

SEQ ID NO: 83 - protein, 7 aa, Artificial Sequence
  vhhLib02-64-CDR2
INSGGST

SEQ ID NO: 84 - protein, 19 aa, Artificial Sequence
  vhhLib02-64-CDR3
ARGRVATGGGYGFVHYFDV

SEQ ID NO: 85 - protein, 131 aa, Artificial Sequence
  vhhLib02-64
EVQLVESGGGLVQPGGSLRLSCAASGFTFSGYFMSWYRQAPGKQRELVSVINSGGSTYYADSVKGRFTISRDNSKNTLYLQMNSLRAEDTAVYYCARGRVATGGGYGFVHYFDVWGQGTLVTVSSHHHHHH

SEQ ID NO: 86 - protein, 8 aa, Artificial Sequence
  vhhLib02-91-CDR1
GFTVSDYH

SEQ ID NO: 87 - protein, 7 aa, Artificial Sequence
  vhhLib02-91-CDR2
ILSGGRT

SEQ ID NO: 88 - protein, 12 aa, Artificial Sequence
  vhhLib02-91-CDR3
ARADAIFPWFDV

SEQ ID NO: 89 - protein, 124 aa, Artificial Sequence
  vhhLib02-91
EVQLVESGGGLVQPGGSLRLSCAASGFTVSDYHMSWYRQAPGKQRELVSVILSGGRTYYADSVKGRFTISRDNSKNTLYLQMNSLRAEDTAVYYCARADAIFPWFDVWGQGTLVTVSSHHHHHH

SEQ ID NO: 90 - protein, 8 aa, Artificial Sequence
  vhhLib02-92-CDR1
GFTFSTYG

SEQ ID NO: 91 - protein, 7 aa, Artificial Sequence
  vhhLib02-92-CDR2
IITGEFT

SEQ ID NO: 92 - protein, 18 aa, Artificial Sequence
  vhhLib02-92-CDR3
AREETYVWDSYYDFVFDL

SEQ ID NO: 93 - protein, 130 aa, Artificial Sequence
  vhhLib02-92
EVQLVESGGGLVQPGGSLRLSCAASGFTFSTYGMSWYRQAPGKQRELVSAIITGEFTYYADSVKGRFTISRDNSKNTLYLQMNSLREEDTAVYYCAREETYVWDSYYDFVFDLWGQGTLVTVSSHHHHHH

SEQ ID NO: 94 - protein, 8 aa, Artificial Sequence
  vhhLib02-93
GFTFSDYH

SEQ ID NO: 95 - protein, 7 aa, Artificial Sequence
  vhhLib02-93
INSGGYT

SEQ ID NO: 96 - protein, 15 aa, Artificial Sequence
  vhhLib02-93
ARAQAAHYLYGYFDI

SEQ ID NO: 97 - protein, 127 aa, Artificial Sequence
  vhhLib02-93
EVQLVESGGGLVQPGGSLRLSCAASGFTFSDYHMSWYRQAPGKQREPVSVINSGGYTYYADSVKGRFTISRDNSKNTLYLQMNSLRAEDTAVYYCARAQAAHYLYGYFDIWGQGTLVTVSSHHHHHH

SEQ ID NO: 98 - protein, 8 aa, Artificial Sequence
  vhhLib05-5-CDR1
GFTFSSYA

SEQ ID NO: 99 - protein, 8 aa, Artificial Sequence
  vhhLib05-5-CDR2
ISGSGGST

SEQ ID NO: 100 - protein, 14 aa, Artificial Sequence
  vhhLib05-5-CDR3
ARLDYTDGINYFDY

SEQ ID NO: 101 - protein, 134 aa, Artificial Sequence
  vhhLib05-5
EVQLLESGGGLVQPGGSLRLSCAASGFTFSSYAMSWVRQAPGKQREWVSAISGSGGSTYYADSVKGRFTISRDNSKNTLYLQMNSLRAEDTAVYYCARLDYTDGINYFDYWGQGTLVTVSSTLVTVSSHHHHHH

SEQ ID NO: 102 - protein, 8 aa, Artificial Sequence
  vhhLib05-8-CDR1
GFTFSSYA

SEQ ID NO: 103 - protein, 8 aa, Artificial Sequence
  vhhLib05-8-CDR2
ISGSGGRT

SEQ ID NO: 104 - protein, 15 aa, Artificial Sequence
  vhhLib05-8-CDR3
ARHSIPVDGIVAFDH

SEQ ID NO: 105 - protein, 135 aa, Artificial Sequence
  vhhLib05-8
EVQLLESGGGLVQPGGSLRLSCAASGFTFSSYAMSWVRQAPGKQREWVSAISGSGGRTYYADSVKGRFTISRDNSKNTLYLQMNSLRAEDTAVYYCARHSIPVDGIVAFDHWGQGTLVTVSSTLVTVSSHHHHHH

SEQ ID NO: 106 - protein, 8 aa, Artificial Sequence
  vhhLib05-10-CDR1
GFTFSSYA

SEQ ID NO: 107 - protein, 8 aa, Artificial Sequence
  vhhLib05-10-CDR2
ISGSGGST

SEQ ID NO: 108 - protein, 15 aa, Artificial Sequence
  vhhLib05-10-CDR3
ARVVKVDDIVHAFDI

SEQ ID NO: 109 - protein, 135 aa, Artificial Sequence
  vhhLib05-10
EVQLLESGGGLVQPGGSLRLSCAASGFTFSSYAMSWVRQAPGKQREWVSAISGSGGSTYYADSVKGRFTISRDNSKNTLYLQMNSLRAEDTAVYYCARVVKVDDIVHAFDIWGQGTLVTVSSTLVTVSSHHHHHH

SEQ ID NO: 110 - protein, 8 aa, Artificial Sequence
  vhhLib05-14-CDR1
GFTFSSYA

SEQ ID NO: 111 - protein, 8 aa, Artificial Sequence
  vhhLib05-14-CDR2
ISGSGGST

SEQ ID NO: 112 - protein, 14 aa, Artificial Sequence
  vhhLib05-14-CDR3
ARLSIVDVVTVFDV

SEQ ID NO: 113 - protein, 134 aa, Artificial Sequence
  vhhLib05-14
EVQLLESGGGLVQPGGSLRLSCAASGFTFSSYAMSWVRQAPGKQREWVSAISGSGGSTYYADSVKGRFTISRDNSKNTLYLQMNSLRAEDTAVYYCARLSIVDVVTVFDVWGQGTLVTVSSTLVTVSSHHHHHH

SEQ ID NO: 114 - protein, 8 aa, Artificial Sequence
  vhhLib05-21-CDR1
GFTFSNYA

SEQ ID NO: 115 - protein, 8 aa, Artificial Sequence
  vhhLib05-21-CDR2
ISGNGSST

SEQ ID NO: 116 - protein, 16 aa, Artificial Sequence
  vhhLib05-21-CDR3
ARLVQLPWVPISAFDV

SEQ ID NO: 117 - protein, 136 aa, Artificial Sequence
  vhhLib05-21
EVQLLESGGGLVQPGGSLRLSCAASGFTFSNYAMSWVRQAPGKQREWVSVISGNGSSTYYADSVKGRFTISRDNSKNTLYLQMNSLRAEDTAVYYCARLVQLPWVPISAFDVWGQGTLVTVSSTLVTVSSHHHHHH

SEQ ID NO: 118 - protein, 8 aa, Artificial Sequence
  vhhLib05-25-CDR1
GFTFSSYA

SEQ ID NO: 119 - protein, 84 aa, Artificial Sequence
  vhhLib05-25-CDR2
ISGSGGSTYYADSVKGRFTISRDNSKNTLYLQMNSLRAEDTAVYYCARHRQADGITYFDIWGQGTLVTVSSTLVTVSSHHHHHH

SEQ ID NO: 120 - protein, 14 aa, Artificial Sequence
  vhhLib05-25-CDR3
ARHRQADGITYFDI

SEQ ID NO: 121 - protein, 134 aa, Artificial Sequence
  vhhLib05-25
EVQLLESGGGLVQPGGSLRLSCAASGFTFSSYAMSWVRQAPGKQREWVSAISGSGGSTYYADSVKGRFTISRDNSKNTLYLQMNSLRAEDTAVYYCARHRQADGITYFDIWGQGTLVTVSSTLVTVSSHHHHHH

SEQ ID NO: 122 - protein, 8 aa, Artificial Sequence
  vhhLib05-28-CDR1
GFTFSSYA

SEQ ID NO: 123 - protein, 8 aa, Artificial Sequence
  vhhLib05-28-CDR2
ISGSGGST

SEQ ID NO: 124 - protein, 18 aa, Artificial Sequence
  vhhLib05-28-CDR3
ARLSHNSQGTVVYNYFDI

SEQ ID NO: 125 - protein, 138 aa, Artificial Sequence
  vhhLib05-28
EVQLLESGGGLVQPGGSLRLSCAAAGFTFSSYAMSWVRQAPGKQREWVSAISGSGGSTYYADSVKGRFTISRDNSKNTLYLQMNSLRAEDTAVYYCARLSHNSQGTVVYNYFDIWGQGTLVTVSSTLVTVSSHHHHHH

SEQ ID NO: 126 - protein, 8 aa, Artificial Sequence
  vhhLib05-33-CDR1
GFTFSSYA

SEQ ID NO: 127 - protein, 8 aa, Artificial Sequence
  vhhLib05-33-CDR2
ISGSGGST

SEQ ID NO: 128 - protein, 18 aa, Artificial Sequence
  vhhLib05-33-CDR3
ARLQGVQSDEVYYNYFDV

SEQ ID NO: 129 - protein, 138 aa, Artificial Sequence
  vhhLib05-33
EVQLLESGGGLVQPGGSLRLSCAASGFTFSSYAMSWVRQAPGKQREWVSAISGSGGSTYYADSVKGRFTISRDNSKNTLYLQMNSLRAEDTAVYYCARLQGVQSDEVYYNYFDVWGQGTLVTVSSTLVTVSSHHHHHH

SEQ ID NO: 130 - protein, 8 aa, Artificial Sequence
  vhhLib05-49-CDR1
GFTFSSYA

SEQ ID NO: 131 - protein, 8 aa, Artificial Sequence
  vhhLib05-49-CDR2
ISGSGGST

SEQ ID NO: 132 - protein, 20 aa, Artificial Sequence
  vhhLib05-49-CDR3
ARDTNQPYHGRYVYTYDFDV

SEQ ID NO: 133 - protein, 140 aa, Artificial Sequence
  vhhLib05-49
EVQLLESGGGLVQPGGSLRLSCAASGFTFSSYAMSWVRQAPGKQREWVSAISGSGGSTYYADSVKGRFTISRDNSKNTLYLQMNSLRAEDTAVYYCARDTNQPYHGRYVYTYDFDVWGQGTLVTVSSTLVTVSSHHHHHH

SEQ ID NO: 134 - protein, 8 aa, Artificial Sequence
  vhhLib05-78-CDR1
GFTFSSYA

SEQ ID NO: 135 - protein, 8 aa, Artificial Sequence
  vhhLib05-78-CDR2
ISGSGGST

SEQ ID NO: 136 - protein, 18 aa, Artificial Sequence
  vhhLib05-78-CDR3
ARLATHVDNGLGYNYFDV

SEQ ID NO: 137 - protein, 138 aa, Artificial Sequence
  vhhLib05-78
EVQLLESGGGLVQPGGSLRLSCAASGFTFSSYAMTWVRQAPGKQREWVSAISGSGGSTYYADSVKGRFTISRDNSKNTLYLQMNSLRAEDTAVYYCARLATHVDNGLGYNYFDVWGQGTLVTVSSTLVTVSSHHHHHH

SEQ ID NO: 138 - protein, 8 aa, Artificial Sequence
  vhhLib05-83-CDR1
GFTFSNYA

SEQ ID NO: 139 - protein, 8 aa, Artificial Sequence
  vhhLib05-83-CDR2
ISGSGGST

SEQ ID NO: 140 - protein, 19 aa, Artificial Sequence
  vhhLib05-83-CDR3
ARLFLQQADGLYYYYAFDP

SEQ ID NO: 141 - protein, 139 aa, Artificial Sequence
  vhhLib05-83
EVQLLESGGGLVQPGGSLRLSCAASGFTFSNYAMSWVRQAPGKQREWVSAISGSGGSTYYADSVKGRFTISRDNSKNTLYLQMNSLRAEDTAVYYCARLFLQQADGLYYYYAFDPWGQGTPVTVSSTLVTVSSHHHHHH

SEQ ID NO: 142 - protein, 94 aa, Artificial Sequence
  vhhLib-01 (Alp_LowDiv) FW
QVQLVESGGGLVQPGGSLRLSCAASGSIFSINAMGWYRQAPGKQRELVAAITSGGSTNYADSVKGRFTISRDNAKNTVYLQMNSLKPEDTAVYY

SEQ ID NO: 143 - protein, 8 aa, Artificial Sequence
  vhhLib-01 (Alp_LowDiv) CDR1
  MISC_FEATURE (2)..(2): X1 at 2 is S or F
  MISC_FEATURE (3)..(3): X2 is I or F
  MISC_FEATURE (6)..(6): X3 = is S, A, C, D, F, G, H, I , L, N, P, R, T, V, Y
  MISC_FEATURE (7)..(7): X4 = is N or Y
  MISC_FEATURE (8)..(8): X5 =is R, A, C, D, F, G, H, I, L, N, P, S, T, V, Y
GXXFSXXX

SEQ ID NO: 144 - protein, 7 aa, Artificial Sequence
  vhhLib-01 (Alp_LowDiv) CDR2
  MISC_FEATURE (2)..(2): X1 is D, A, C, F, G, H, I, L, N, P, R, S, T, V or y
  MISC_FEATURE (3)..(3): X2 is S or R
  MISC_FEATURE (4)..(4): X3 is G or W
  MISC_FEATURE (6)..(6): X4 is Y, A, C, F, G, H, I, L, N, P, R, S, T or V
IXXXGXT

SEQ ID NO: 145 - protein, 31 aa, Artificial Sequence
  VHHLIB-01 (ALP_LOWDIV) CDR3
  MISC_FEATURE (3)..(3): X1 is = D, E, G, H, L, Q, R, S V or Y
  MISC_FEATURE (4)..(4): X2 is = D, Q, R, T
  MISC_FEATURE (5)..(5): X3 is = V
  MISC_FEATURE (6)..(8): X= any amino acid
  MISC_FEATURE (9)..(9): X7=T
  MISC_FEATURE (10)..(10): X8= D, F, G, Q, R, V OR Y
  MISC_FEATURE (11)..(11): X9=A, D, E, F, G, H, I, K, L, N, P, Q, R, S, T, V or W
  MISC_FEATURE (15)..(15): X10 = A, D, E, F, G, H, I, K, L,, N,  Q, R, S, T, V or Y,
  MISC_FEATURE (16)..(16): X11= A, D, E, F, G, H, I, L, Q, R, S, T, V or Y,
  MISC_FEATURE (17)..(17): X12= A, D  F, G, H, K, L, N,  R, S, T, V, W or Y,
  MISC_FEATURE (18)..(18): X13= A, D, E  F, G, H, I, L, N, Q, R,  S, T, V, W or Y,
  MISC_FEATURE (19)..(19): X14= , D, E  F, G, H, I, L, N, P,  R,  S, T, V, W or Y,
  MISC_FEATURE (20)..(20): X15= A,D,E,F,G,H,I,L, N, P, R,S,T,V,W,Y
  MISC_FEATURE (21)..(21): X16= A,D,E,F,G,H,I,K,L,Q,R,S,T,V,W,Y
  MISC_FEATURE (22)..(22): X17= A,D,E,F,G,H,I,K,L, N, Q,R,S,T,V,W,Y
  MISC_FEATURE (24)..(24): X18= Q,Y
  MISC_FEATURE (25)..(25): X19= Any Amino Acids
  MISC_FEATURE (26)..(26): X20 =  Y
  MISC_FEATURE (27)..(27): X21=G
  MISC_FEATURE (28)..(28): X22=A, G, L, P, S, W, Y
  MISC_FEATURE (29)..(29): X23=  F, C, L, S, V, or Y
  MISC_FEATURE (30)..(30): X24= D, A, E, G, T or  V
  MISC_FEATURE (31)..(31): X25= V, I, P, S or Y
ARXXXXXXXXXYDDXXXXXXXXYXXXXXXXX

SEQ ID NO: 146 - protein, 94 aa, Artificial Sequence
  vhhLib-02 (Hum_LowDiv) FW
EVQLVESGGGLVQPGGSLRLSCAASGFTFSSYAMSWYQQAPGKQRELVSAITSGGSTYYADSVKGRFTISRDNSKNTLYLQMNSLRAEDTAVYY

SEQ ID NO: 147 - protein, 8 aa, Artificial Sequence
  vhhLib-02 (Hum_LowDiv) CDR1
  MISC_FEATURE (2)..(2): X1=F or l
  MISC_FEATURE (4)..(4): X2=F OR V
  MISC_FEATURE (6)..(6): X3= D, A, C, F, G, H, I, L, P, R, S, T, V, OR Y
  MISC_FEATURE (8)..(8): X4=H, A, C, D, F, G, I, L, P, R, S, T, V, OR Y
GXTXSXYX

SEQ ID NO: 148 - protein, 7 aa, Artificial Sequence
  vhhLib-02 (Hum_LowDiv) CDR2
  MISC_FEATURE (1)..(1): X1 = I or T
  MISC_FEATURE (2)..(2): X2 = L, A, C, D, F, H, I, N, S, T, V, or Y
  MISC_FEATURE (3)..(3): X3 = S, C, R, or T
  MISC_FEATURE (4)..(4): X4 = G, A, C, D, or VT
  MISC_FEATURE (5)..(5): X5 = G or E
  MISC_FEATURE (6)..(6): X6  = R, A, D, F, G, H, I, L, N, P, S, T, V or Y
XXXXXXT

SEQ ID NO: 149 - protein, 27 aa, Artificial Sequence
  vhhLib-02 (Hum_LowDiv) CDR3
  MISC_FEATURE (3)..(3): X1 = D, E, G, L, R, S, T, V, or Y
  MISC_FEATURE (4)..(4): X2 = D, F, G, L, N, Q, R, S, T, V, or Y
  MISC_FEATURE (5)..(5): X3 = A, F, G, Q, or VY
  MISC_FEATURE (6)..(7): X4 and X5 = dash with no amino acid below can be any naturally occurring amino acid, or a gap
  MISC_FEATURE (8)..(8): X6 = gap or A, D, E, G, H, I, L, Q, R, T, V or Y
  MISC_FEATURE (9)..(9): X7 = gap or D, E, F, H, I L, N, Q, R, S, T, V, W, or Y
  MISC_FEATURE (10)..(10): X8 = gap or D,F, G, H, I L, P, Q, R, S, T, V, W or Y
  MISC_FEATURE (11)..(11): X9 = gap or A, D, E, G, G, H, I or L
  MISC_FEATURE (12)..(12): X10 = gap, or G, H, L, R, S, T, W or Y
  MISC_FEATURE (13)..(13): X11 = gap or L
  MISC_FEATURE (14)..(14): X12 = gap, or A, D, E, G, I, K, L, P, Q, R, S, T or Y
  MISC_FEATURE (15)..(15): X13= gap or G, T, or V
  MISC_FEATURE (16)..(16): X14 = gap or A, D, E, G, I, K, L, P, Q, R, S, T or Y
  MISC_FEATURE (17)..(17): X15 = , D, A, E, G, G, H, I, K, L , N, P, Q, R, S, or T
  MISC_FEATURE (18)..(18): X16 = D, A, E, G, G, H, I, K, L, N, P, Q, R, S or T
  MISC_FEATURE (19)..(29): X17 = A, D, E, G, G, H, I, L, P, Q, R, S, T, V, or Y
  MISC_FEATURE (20)..(20): X18 = Y, D, D, F, G, H, I, L, , N, Q, R, S, T, V or W
  MISC_FEATURE (21)..(21): X19 = gap, or A, D, G, H, S or Y
  MISC_FEATURE (22)..(22): X20 = gap, or D, E, F, I, L or R
  MISC_FEATURE (23)..(23): X21 = gap or A, , C, D, E, F, G, H, I, L, N, P, Q, R or S
  MISC_FEATURE (24)..(24): X22 = Y, A, C, D, G, H, I, L, P, R, S, T, V, or W
  MISC_FEATURE (25)..(25): X23 =F, L, V or Y
  MISC_FEATURE (26)..(26): X24 = D, A, E, G, T or V
  MISC_FEATURE (27)..(27): X25 = V, A, P or Y
ARXXXXXXXXXXXXXXXXXXXXXXXXX

SEQ ID NO: 150 - protein, 95 aa, Artificial Sequence
  vhhLib-05 (Hum_HighDiv) FW
  misc_feature (50)..(50): Xaa can be any naturally occurring amino acid
EVQLLESGGGLVQPGGSLRLSCAASGFTFSSYAMSWVRQAPGKQREWVSXISGSGGSTYYADSVKGRFTISRDNSKNTLYLQMNSLRAEDTAVYY

SEQ ID NO: 151 - protein, 8 aa, Artificial Sequence
  vhhLib-05 (Hum_HighDiv) CDR1
  MISC_FEATURE (5)..(5): X1=S, C, G, N, R OR T
  MISC_FEATURE (6)..(6): X2-S, G, N, R OR T
GFTFXXYA

SEQ ID NO: 152 - protein, 9 aa, Artificial Sequence
  vhhLib-05 (Hum_HighDiv) CDR2
  MISC_FEATURE (1)..(1): X1 = A, G, P, S, T or V
  MISC_FEATURE (4)..(4): X2 = G, A, R, S or V
  MISC_FEATURE (5)..(5): X3 = S, C, G, I, N, R or T
  MISC_FEATURE (6)..(6): X4 = G or D
  MISC_FEATURE (7)..(7): X5 = G, A, D, R, S or V
  MISC_FEATURE (8)..(8): X6 = S, G, I, N, R or T
XISXXXXXT

SEQ ID NO: 153 - protein, 31 aa, Artificial Sequence
  VHHLIB-05 (HUM_HIGHDIV) CDR3
  MISC_FEATURE (3)..(3): X1 = gap or A, D, E, G, H, L, P, Q, R, T, V or Y
  MISC_FEATURE (4)..(4): X2 = gap or D, E, G, H, K, L, N, Q, R, S, T, V or Y
  MISC_FEATURE (5)..(5): X3= gap or A, D< F, G, I, K, L, N, Q, R, S, T, V, W or Y
  MISC_FEATURE (5)..(5): X3= gap or A, D, F, G, I, K, L, N, Q, R, S, T, V, W or Y
  MISC_FEATURE (6)..(6): X4= gap or A, R, or S
  MISC_FEATURE (7)..(7): X5= gap or D, G, L, N, Q, R, S or V
  MISC_FEATURE (8)..(8): X6= gap or D, E, G, H, L, P, R, S< T, V, or Y
  MISC_FEATURE (9)..(9): X7= gap or D, G, H, L, R, T, V or Y
  MISC_FEATURE (10)..(10): X8= gap or A, D, E, G, H, I , K L, N, Q, R, S, T, V, W or Y
  MISC_FEATURE (11)..(11): X9= gap or D, G, H, I, K, L, N, P, Q, R, S, T, V or Y
  MISC_FEATURE (12)..(12): X10= gap or A, D, F, G, H, I, L, N, Q, R, S, T, V, W or Y
  MISC_FEATURE (13)..(13): X11= gap or A, D, E, F, G, H, I, K, L, N, Q, R, S, T, V, W  or Y
  MISC_FEATURE (14)..(14): X12= gap or A, D, E, G, H, I, L, N, P, Q, R, S, T, V, W OR Y
  MISC_FEATURE (15)..(15): X13= gap or a, D, E, F, G, H, I, K, L, N, P, Q, R, S, Y, V, W, or Y
  MISC_FEATURE (16)..(16): X14= gap or A, D, E, F, G, H, I, L, N, Q, R, S, T, W or Y
  MISC_FEATURE (17)..(17): X15= gap or A, D, E, F, G, H, I, L, N, Q, Q, R, S, T, V, W or Y
  MISC_FEATURE (18)..(18): X16 = gap or A, D, E, F, G, H, I, L, N, P, Q, R, S, T, V, W  or Y
  MISC_FEATURE (19)..(19): X17 = gap or A, D, E, F, G, H, I, K, N, R, S, T, V, W OR Y
  MISC_FEATURE (20)..(20): X18 = any amino acid or gap
  MISC_FEATURE (21)..(21): X19 = any amino acid or gap
  MISC_FEATURE (22)..(22): X20 = any amino acid or gap
  MISC_FEATURE (23)..(23): X21 = any amino acid or gap
  MISC_FEATURE (24)..(24): X22 = gap or D or Y
  MISC_FEATURE (25)..(25): X23 = gap or F, I, L or Y
  MISC_FEATURE (26)..(26): X24 = gap or A, D, E, F, G, H, I, K, L, N, Q, R, S, T, V, W OR Y
  MISC_FEATURE (27)..(27): X25 = Y, A, D, G, H, I, L, P, T, V or M
  MISC_FEATURE (28)..(28): X26= F or L
  MISC_FEATURE (29)..(29): X27= D or T
  MISC_FEATURE (30)..(30): X28 = V, I, P or Y
  misc_feature (31)..(31): Xaa can be any naturally occurring amino acid
ARXXXXXXXXXXXXXXXXXXXXXXXXXXLXX

SEQ ID NO: 154 - protein, 42 aa, Homo sapiens [sequence omitted]

SEQ ID NO: 155 - protein, 40 aa, Homo sapiens
DAEFRHDSGYEVHHQKLVFFAEDVGSNKGAIIGLMVGGWI

SEQ ID NO: 156 - protein, 39 aa, Homo sapiens
DAEFRHDSGYEVHHQKLVFFAEDVGSNKGAIIGLMVGGW

SEQ ID NO: 157 - protein, 39 aa, Homo sapiens
DAEFRHDSGYEVHHQKLVFFAEDVGSNKGAIIGLMVGGV

SEQ ID NO: 158 - protein, 43 aa, Homo sapiens
DAEFRHDSGYEVHHQKLVFFAEDVGSNKGAIIGLMVGGVVIAT